(19) United States
(12) Patent Application Publication
(10) Pub. No.: US 2026/0306255 A1
(43) Pub. Date: Oct. 8, 2026

(54) Atrioventricular Valve Modification Systems, Devices, and Methods

(71) Applicant: Evalve, Inc., Santa Clara, CA (US)

(72) Inventors: Saurabh Datta, Pleasanton, CA (US); Preston James Huddleston, St. Paul, MN (US); Caitlyn E. Harrington, San Francisco, CA (US); Clara Park, Santa Clara, CA (US); Cynthia Siu, Belmont, CA (US); Taylor Nasby, Savage, MN (US)

(73) Assignee: Evalve, Inc., Santa Clara, CA (US)

(21) Appl. No.: 19/632,914

(22) Filed: Mar. 30, 2026

Related U.S. Application Data
(60) Provisional application No. 63/783,882, filed on Apr. 4, 2025.

(51) Int. Cl. A61F 2/24; A61N 1/05; A61N 1/375

(52) U.S. Cl. CPC A61F 2/2418; A61F 2/2427; A61N 1/0573; A61N 2001/058; A61N 1/37518

(57) ABSTRACT

A method of implanting a prosthetic atrioventricular valve into a native atrioventricular valve includes delivering a fixation device to the native valve, and applying the fixation device to engage and approximate free edges of two leaflets of the native valve. After applying the fixation device, the prosthetic valve is delivered to the native valve while the prosthetic valve is in a collapsed condition. The prosthetic valve is then be deployed into the native valve so that the prosthetic valve expands into engagement with the native valve. A transcatheter positioning device for an atrioventricular valve modification device delivery system is also provided. The positioning device comprises a positioning member having an undeployed configuration for delivery of the positioning device to an atrioventricular valve modification device delivery site in the heart of a patient and a deployed configuration for the positioning member to engage and reposition an object in the heart.

### BACKGROUND OF THE DISCLOSURE

The cardiac cycle is divided into two phases—diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve, and the opening of the aortic and pulmonary valve. The mitral valve separates the left atrium from the left ventricle, and the tricuspid valve separates the right atrium from the right ventricle. The aortic valve separates the left ventricle from the aorta, and the pulmonary valve separates the right ventricle from the pulmonary artery. The mitral and tricuspid valves operate as check valves preventing blood from flowing back into the atria during ventricular contraction. The aortic and pulmonary valves operate as check valves preventing blood from flowing back into the ventricles during ventricular relaxation.

All of the heart valves may be susceptible to disease. For example, valvular insufficiency may appear in one or both of the mitral and tricuspid valves which may result in a regurgitative flow back into the atrium across the affected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrythmias, pulmonary hypertension, stroke, and endocarditis, to name a few.

Treatment options for MVR may include replacement and/or repair of the mitral valve, which may include open-heart surgical options and catheter-based options. Catheter-based repair procedures are sometimes referred to as transcatheter edge-to-edge repair (“TEER”).

### BRIEF SUMMARY OF THE DISCLOSURE

Although transcatheter replacement of the mitral valve or the tricuspid valve may be effective procedures to treat regurgitant native valves, there are many reasons that certain patients may not be ideal candidates for such treatments. For example, patients with regurgitant tricuspid valves may have a very large tricuspid valve annulus and/or complex annular geometry, and because prosthetic heart valves are typically manufactured in a limited number of sizes and/or shapes, there may not be an existing prosthetic heart valve that will suitably fit in the very large and/or complexly-shaped annulus of the patient. Some patients that would benefit from a replacement prosthetic mitral or tricuspid valve already have a pacemaker implanted, which may include a pacemaker lead extending through the native heart valve. Similarly, some patients may require a pacemaker implantation as an adjunct to a prosthetic heart valve implantation. However, the existence of pacemaker leads crossing the native annulus of the patient may create difficulties in properly positioning a transcatheter prosthetic heart valve in that native annulus. Additionally, for some patients, a transcatheter prosthetic heart valve may be implanted such that the delivery device and/or prosthesis contacts electrical conduction centers within the heart, which may result in the need for a pacemaker that might not otherwise be needed. Still further, a patient in need of a prosthetic mitral valve may be deemed an unsuitable candidate for a transcatheter prosthetic mitral valve replacement if the prosthesis is likely to be positioned such that it interferes with suitable blood flow through the left ventricular outflow tract (“LVOT”). At least some of these potential issue may be solved or otherwise mitigated with the use of an accessory implant, such as a leaflet fixation device, to modify the native anatomy and/or to provide structure to allow for more patients to be treated, or for patients to be more effectively treated, with a transcatheter prosthetic tricuspid or mitral valve.

According to one aspect of the disclosure, a method of implanting a prosthetic atrioventricular valve into a native atrioventricular valve of a patient includes delivering a fixation device to the native atrioventricular valve, and applying the fixation device to engage and approximate free edges of two native leaflets of the native atrioventricular valve. After applying the fixation device, the prosthetic atrioventricular valve is delivered to the native atrioventricular valve while the prosthetic atrioventricular valve is in a collapsed condition. The prosthetic atrioventricular valve is then deployed into the native atrioventricular valve so that the prosthetic atrioventricular valve expands into engagement with the native atrioventricular valve. Prior to applying the fixation device, a native annulus of the native atrioventricular valve may have an initial size, and after applying the fixation device, the native annulus of the native atrioventricular valve may have a modified size, the modified size being smaller than the initial size. The prosthetic atrioventricular valve may be self-expandable and may have an operational size in the absence of applied forced, the operational size of the prosthetic atrioventricular valve being too small to anchor to the native annulus of the atrioventricular valve when the native annulus has the initial size, but being sufficiently large to anchor to the native annulus of the atrioventricular valve when the native annulus has the modified size. The native atrioventricular valve may be a native tricuspid valve, and applying the fixation device may include applying a first fixation device to engage and approximate free edges of a first native leaflet and a second native leaflet of the native tricuspid valve, and applying a second fixation device to engage and approximate free edges of the first native leaflet and a third native leaflet of the native tricuspid valve. The prosthetic atrioventricular valve may include a frame that has a waist portion, an atrial anchor flaring radially outward from the waist portion, and a ventricular anchor flaring radially outward from the waist portion, and deploying the prosthetic atrioventricular valve into the native atrioventricular valve may include positioning the fixation device in a space between the atrial anchor and the ventricular anchor of the frame. The steps of applying the fixation device and deploying the prosthetic atrioventricular valve may be performed as part of a single procedure performed in the same day.

According to another aspect of the disclosure, a method of implanting a prosthetic atrioventricular valve into a native atrioventricular valve of a patient includes delivering a first fixation device to the native atrioventricular valve, applying the first fixation device to engage and approximate free edges of two native leaflets of the native atrioventricular valve, delivering a second fixation device to the native atrioventricular valve, applying the second fixation device to engage and approximate free edges of two native leaflets of the native atrioventricular valve, delivering the prosthetic atrioventricular valve to the native atrioventricular valve while the prosthetic atrioventricular valve is in a collapsed condition, and deploying the prosthetic atrioventricular valve into the native atrioventricular valve by inflating a balloon over which the native atrioventricular valve is crimped to expand the prosthetic atrioventricular valve against the first fixation device and against the second fixation device to anchor the prosthetic atrioventricular valve within the native atrioventricular valve. The native atrioventricular valve may be a native mitral valve, and applying the first fixation device may include engaging and approximating free edges of a native anterior leaflet and a native posterior leaflet of the native mitral valve at a first location, and applying the second fixation device may include engaging and approximating free edges of the native anterior leaflet and the native posterior leaflet of the native mitral valve at a second location, the first location being closer to an anterolateral commissure of the native mitral valve than is the second location, and the second location being closer to a posteromedial commissure of the native mitral valve than is the first location. The native atrioventricular valve may be a native tricuspid valve, and the two native leaflets engaged and approximated by the first fixation device may be a first pair selected from (i) a first leaflet of the native tricuspid valve, (ii) a second leaflet of the native tricuspid valve, and (iii) a third leaflet of the native tricuspid valve, and the two native leaflets engaged and approximated by the second fixation device may be a second pair selected from (i) the first leaflet of the native tricuspid valve, (ii) the second leaflet of the native tricuspid valve, and (iii) the third leaflet of the native tricuspid valve, the first pair being different than the second pair. The method may include delivering a third fixation device to the native tricuspid valve, and applying the third fixation device to engage and approximate free edges of two native leaflets of the native tricuspid valve. The two native leaflets engaged and approximated by the third fixation device may be a third pair selected from (i) the first leaflet of the native tricuspid valve, (ii) the second leaflet of the native tricuspid valve, and (iii) the third leaflet of the native tricuspid valve, the third pair being different than both the first pair and the second pair. The steps of applying the first fixation device, applying the second fixation device, and deploying the prosthetic atrioventricular valve may be performed as part of a single procedure performed in the same day.

According to a further aspect of the disclosure, a method of implanting a prosthetic tricuspid valve into a native tricuspid valve of a patient includes delivering a fixation device to the native tricuspid valve, applying the fixation device to engage and approximate free edges of (i) a septal leaflet of the native tricuspid valve and (ii) a second leaflet of the native tricuspid valve, the second leaflet being either (a) an anterior leaflet of the native tricuspid valve or (b) a posterior leaflet of the native tricuspid valve, after applying the fixation device, delivering the prosthetic tricuspid valve to the native tricuspid valve while the prosthetic tricuspid valve is in a collapsed condition, and deploying the prosthetic tricuspid valve into the native tricuspid valve by expanding the prosthetic tricuspid valve into engagement with the native tricuspid valve, such that no structure of the prosthetic tricuspid valve contacts an atrioventricular (“AV”) node of the patient. Applying the fixation device may include applying a first fixation device to engage and approximate free edges of (i) the septal leaflet of the native tricuspid valve and (ii) the anterior leaflet of the native tricuspid valve, and applying a second fixation device to engage and approximate free edges of (i) the septal leaflet of the native tricuspid valve and (ii) the posterior leaflet of the native tricuspid valve. The steps of applying the fixation device and deploying the prosthetic tricuspid valve may be performed as part of a single procedure performed in the same day.

According to another aspect of the disclosure, a method of implanting a prosthetic mitral valve into a native mitral valve of a patient includes delivering a fixation device to the native mitral valve, applying the fixation device to engage and approximate free edges of (i) an anterior leaflet of the native mitral valve and (ii) a posterior leaflet of the native mitral valve, after applying the fixation device, delivering the prosthetic mitral valve to the native mitral valve while the prosthetic mitral valve is in a collapsed condition, and deploying the prosthetic mitral valve into the native mitral valve by expanding the prosthetic mitral valve into engagement with the native mitral valve, such that no structure of the prosthetic mitral valve obstructs blood flowing through a left ventricular outflow tract (“LVOT”) of the patient. The steps of applying the fixation device and deploying the prosthetic mitral valve may be performed as part of a single procedure performed in the same day.

A further aspect of the disclosure is directed to a method of implanting a prosthetic atrioventricular valve into a native atrioventricular valve of a heart of a patient, wherein the patient has a pacemaker lead extending from an atrium of the heart to a ventricle of the heart through the native atrioventricular valve. The method includes delivering a fixation device to the native atrioventricular valve, applying the fixation device to engage and approximate free edges of two native leaflets of the native atrioventricular valve so that a portion of the pacemaker lead becomes trapped between the fixation device and an adjacent commissure of the native atrioventricular valve, while the pacemaker lead is trapped, delivering the prosthetic atrioventricular valve to the native atrioventricular valve while the prosthetic atrioventricular valve is in a collapsed condition, and deploying the prosthetic atrioventricular valve into the native atrioventricular valve so that the prosthetic atrioventricular valve expands into engagement with the native atrioventricular valve. The native atrioventricular valve may be a native tricuspid valve, the two native leaflets engaged and approximated by the fixation device may be a septal leaflet and a posterior leaflet of the native tricuspid valve, and the commissure may be a commissure connecting the septal leaflet to the posterior leaflet. The steps of applying the fixation device and deploying the prosthetic atrioventricular valve may be performed as part of a single procedure performed in the same day. The method may include implanting a pacemaker into the patient so that the pacemaker lead extends from the atrium to the ventricle through the native atrioventricular valve, and the steps of (i) implanting the pacemaker, (ii) applying the fixation device, and (iii) deploying the prosthetic atrioventricular valve may be performed as part of a single procedure performed in the same day.

According to another aspect of the disclosure, a method of implanting a prosthetic atrioventricular valve into a native atrioventricular valve of a heart of a patient includes delivering the prosthetic atrioventricular valve to the native atrioventricular valve while the prosthetic atrioventricular valve is in a collapsed condition, and deploying the prosthetic atrioventricular valve into the native atrioventricular valve so that the prosthetic atrioventricular valve expands into engagement with the native atrioventricular valve, wherein deploying the prosthetic atrioventricular valve includes (i) positioning a first anchor arm of the prosthetic atrioventricular valve in a first gap space between (a) a first fixation device that engages and approximates free edges of two native leaflets of the native atrioventricular valve and (b) a first native commissure adjacent to the fist fixation device, and (ii) positioning a second anchor arm of the prosthetic atrioventricular valve in a second gap space between (a) a second fixation device that engages and approximates free edges of two native leaflets of the native atrioventricular valve and (b) a second native commissure adjacent to the second fixation device. After the prosthetic atrioventricular valve is deployed, the first anchor arm may include a first base connected to a ventricular end portion of the prosthetic atrioventricular valve, a first terminal end, and a first middle portion between the first base and the first terminal end. After the prosthetic atrioventricular valve is deployed, the first base of the first anchor arm may be positioned on an outflow side of the native atrioventricular valve, the first terminal end of the first anchor arm may be positioned on an inflow side of the native atrioventricular valve, and the first middle portion of the first anchor arm may be positioned within the first gap space. After the prosthetic atrioventricular valve is deployed, the first terminal end of the first anchor arm may be in contact with the first fixation device. Prior to delivering the prosthetic atrioventricular valve to the native atrioventricular valve, the first fixation device may be delivered to the native atrioventricular valve and the first fixation device may be applied to engage and approximate free edges of two native leaflets of the native atrioventricular valve, and the second fixation device may be delivered to the native atrioventricular valve and the second fixation device may be applied to engage and approximate free edges of two native leaflets of the native atrioventricular valve. The steps of (i) applying the first fixation device, (ii) applying the second fixation device, and (iii) deploying the prosthetic atrioventricular valve may be performed as part of a single procedure performed in the same day. After positioning the first anchor arm of the prosthetic atrioventricular valve in the first gap space, an occluder may be delivered to the native atrioventricular valve and the occluder mya be deployed within the first gap space.

According to a further aspect of the disclosure, there is provided a transcatheter positioning device for an atrioventricular valve modification device delivery system, the positioning device comprising a positioning member having an undeployed configuration for delivery of the positioning device to an atrioventricular valve modification device delivery site in the heart of a patient, and a deployed configuration for the positioning member to engage and reposition an object in the heart.

According to a further aspect of the disclosure, there is provided a method of delivering a transcatheter atrioventricular valve modification device to an atrioventricular valve modification device delivery site in the heart of a patient, the method comprising delivering a transcatheter positioning device comprising a positioning member in an undeployed configuration to the delivery site; deploying the positioning member to a deployed configuration; and engaging and repositioning an object in the heart.

According to a further aspect of the disclosure, there is provided an atrioventricular valve repair system comprising a first fixation device comprising first fixation elements configured to engage first heart tissue, the first heart tissue comprising one or more of a) first edge portions of two native leaflets of a native atrioventricular valve, the first edge portions being adjacent a first commissure defined between the two native leaflets, b) one or more chordae tendineae, c) a papillary muscle, and/or d) a moderator band; and a second fixation device comprising second fixation elements configured to engage second heart tissue, the second heart tissue comprising second edge portions of two native leaflets of the native atrioventricular valve, the second edge portions being located away from the first commissure; wherein the first fixation device is smaller than the second fixation device.

According to a further aspect of the disclosure, there is provided a method of modifying a native atrioventricular valve of a patient, the method comprising: delivering a first fixation device to the native atrioventricular valve, the first fixation device comprising first fixation elements; applying the first fixation elements to engage first edge portions of two native leaflets of the native atrioventricular valve, the first edge portions being adjacent a first commissure defined between the two native leaflets; delivering a second fixation device to the native atrioventricular valve, the second fixation device comprising second fixation elements; applying the second fixation elements to engage second edge portions of two native leaflets of the native atrioventricular valve, the second edge portions being located away from the first commissure.

According to a further aspect of the disclosure, there is provided a method of modifying a native atrioventricular valve of a patient, the native atrioventricular valve having a pacemaker lead or leads passing therethrough from the atrium associated with the atrioventricular valve to the ventricle associated with the atrioventricular valve, the method comprising: delivering a first fixation device to the ventricle, the first fixation device comprising first fixation elements; and applying the first fixation elements to engage the lead or leads of the pacemaker along with one or more of a) one or more chordae tendineae, b) a papillary muscle, and/or c) a moderator band in the ventricle, to secure the lead or leads of the pacemaker thereto.

According to a further aspect of the disclosure, there is provided a delivery system for delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the delivery system comprising: a guide catheter configured to pass through the patient's vasculature towards the delivery site, the guide catheter comprising: a first lumen for passage therethrough of a delivery catheter for delivery of an atrioventricular valve modification device to the delivery site; a first steerable portion configured to deflect the guide catheter in the atrium from a first orientation not parallel to the inflow axis to a second orientation substantially parallel to the inflow axis for the guide catheter to pass through the atrioventricular valve from the atrium to the ventricle; and a second steerable portion configured to deflect the guide catheter in the ventricle from the second orientation to a third orientation not parallel to the inflow axis.

According to a further aspect of the disclosure, there is provided a method of delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the method comprising: delivering a guide catheter through the patient's vasculature to the patient's heart; deflecting a first steerable portion of the guide catheter in the atrium from a first orientation not parallel to the inflow axis to a second orientation substantially parallel to the inflow axis; delivering the guide catheter through the atrioventricular valve from the atrium to the ventricle; and deflecting a second steerable portion of the guide catheter in the ventricle from the second orientation to a third orientation not parallel to the inflow axis.

According to a further aspect of the disclosure, there is provided a delivery system for delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the delivery system comprising: a first delivery catheter configured to pass through the patient's vasculature to the delivery site, the first delivery catheter comprising a first steerable delivery catheter portion configured to deflect the first delivery catheter in the ventricle from a first orientation substantially parallel to the inflow axis to a second orientation not parallel to the inflow axis for the first delivery catheter to be directed towards first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

According to a further aspect of the disclosure, there is provided a method of delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the method comprising: delivering a first delivery catheter through the atrioventricular valve from the atrium to the ventricle in a first orientation substantially parallel to the inflow axis; and deflecting a first steerable delivery catheter portion of the first delivery catheter in the ventricle from the first orientation to a second orientation not parallel to the inflow axis for the first delivery catheter to be directed towards first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

According to a further aspect of the disclosure, there is provided a method of modifying a native atrioventricular valve, the method comprising: modifying the atrioventricular valve using a first transcatheter atrioventricular valve modification device; modifying the atrioventricular valve using a second transcatheter atrioventricular valve modification device; wherein the first transcatheter atrioventricular valve modification device differs from the second transcatheter atrioventricular valve modification device.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2 is a side view of an example of a prosthetic atrioventricular valve for use in a transcatheter atrioventricular valve replacement procedure.

FIG. 3 is a perspective view of an example of a fixation device for use in a TEER procedure.

FIG. 4A-1 is a schematic en face view of a heart with fixation devices applied to leaflets of the tricuspid valve.

FIG. 4A-2 is a schematic en face view showing the view of FIG. 4A-1 prior to the application of the fixation devices to the leaflets.

FIG. 4A-3 is a schematic en face view showing the modification of the tricuspid valve annulus between the view of FIG. 4A-2 and FIG. 4A-1.

FIG. 4B is a schematic en face view of a prosthetic heart valve implanted into the tricuspid valve shown in FIG. 4A-1.

FIG. 4C is a schematic lateral view of the prosthetic heart valve shown in FIG. 4B implanted into the tricuspid valve shown in FIG. 4A-1.

FIG. 4D is a flowchart showing example steps of a prosthetic heart valve implantation procedure.

FIG. 5 is a flowchart showing example steps of a prosthetic heart valve implantation procedure.

FIG. 6A is a schematic en face view of a heart with a fixation device applied to leaflets of the mitral valve and a prosthetic heart valve implanted into the mitral valve.

FIG. 6B is a schematic lateral view of the prosthetic heart valve shown in FIG. 6A implanted into the mitral valve shown in FIG. 6A.

FIG. 6C is a flowchart showing example steps of a prosthetic heart valve implantation procedure.

FIG. 7A is a schematic en face view of a heart with a pacemaker lead extending through the tricuspid valve and a fixation device applied to leaflets of the tricuspid valve.

FIG. 7B is a schematic lateral view of a prosthetic heart valve implanted into the tricuspid valve shown in FIG. 7A.

FIG. 7C is a flowchart showing example steps of a prosthetic heart valve implantation procedure.

FIG. 8A is a highly schematic illustration of a cross-section of a prosthetic mitral valve implanted into a mitral valve having fixation devices applied thereto.

FIG. 8B is a schematic illustration of a cross-section of the prosthetic mitral valve of FIG. 8A in an expanded condition.

FIG. 8C is a side view of an occluder in an expanded condition.

FIG. 8D is a flowchart showing example steps of a prosthetic heart valve implantation procedure.

FIG. 9 is a schematic view of a tricuspid valve repair using first and second fixation devices.

FIG. 10 is a schematic view of a mitral valve repair using first and second fixation devices.

FIG. 11 is a flowchart showing an example method of modifying a native atrioventricular valve.

FIG. 12 is a flowchart showing another example method of modifying a native atrioventricular valve.

FIG. 13 is a schematic view showing a first fixation device used to secure a lead of a pacemaker to heart tissue in the right ventricle.

FIG. 14 is a flowchart showing an example method of modifying a native atrioventricular valve to arrive at the configuration of FIG. 13.

FIG. 15 is a side view of an exemplary fixation device for engaging and grasping heart tissue.

FIG. 16 is a side view of another exemplary fixation device for engaging and grasping heart tissue.

FIG. 17 is a schematic top view of fixation elements of a fixation device in an open configuration.

FIG. 18 is a schematic side view of an example of gripping arms of a fixation device.

FIG. 19 is a schematic side view of another example of gripping arms of a fixation device.

FIG. 20 is a schematic side view of another example of gripping arms of a fixation device.

FIG. 21 is a top view of the gripping arms of FIG. 20.

FIG. 22 is a schematic cross-sectional view of a coupling member in an uncoupled configuration.

FIG. 23 is a schematic cross-sectional view of a coupling member in a coupled configuration.

FIG. 24 is a schematic cross-sectional view of a heart showing deployment of a fixation device delivery system.

FIG. 25 is a schematic cross-sectional view of the left side of the heart showing an example delivery of a first fixation device to the left ventricle.

FIG. 26 is a schematic cross-sectional view of the left side of the heart showing another example delivery of a first fixation device to the left ventricle.

FIG. 27 is a flowchart showing an example method for arriving at the configuration shown in FIG. 25.

FIG. 28 is a flowchart showing an example method for arriving at the configuration shown in FIG. 26.

FIG. 29 is a schematic side view of a positioning device having a hook shaped positioning member.

FIG. 30 is a schematic side view of a catheter for deploying the positioning device of FIG. 29.

FIGS. 31A-C are schematic views of a positioning device having a loop shaped positioning member.

FIGS. 32A-B are schematic views of a positioning device having a coil shaped positioning member.

FIGS. 33A-B are schematic views of a positioning member provided by an intermediate portion of a positioning device.

FIGS. 34A-B are schematic side and top-down views of a positioning device having two cooperating positioning members.

FIGS. 35A-B are schematic side and top-down views of a positioning device having two cooperating positioning members using magnets.

FIGS. 36A-B are schematic side and top-down views of a positioning device having two intersecting positioning members.

FIGS. 37A-C are schematic views of a positioning device having a positioning member passing through an opening in a fixation device.

FIGS. 38A-C are schematic views of a positioning device having four positioning members cooperating with a fixation device.

FIG. 39 is a schematic cross-sectional view of a guide catheter for housing a positioning device.

FIG. 40 is a schematic cross-sectional view of a known end member for a delivery catheter.

FIG. 41 is a schematic cross-sectional view of a known delivery catheter.

FIG. 42 is a schematic cross-sectional view of a delivery catheter for housing a positioning device.

FIG. 43 is a schematic side view of a delivery catheter for housing a positioning device, the catheter being releasably coupled to a fixation device.

FIG. 44 is a schematic side view of a fixation device delivery system including a positioning system.

FIG. 45 is a schematic side view of a fixation device delivery system including another positioning system.

FIG. 46 is a schematic side view of a fixation device delivery system including another positioning system.

FIG. 47 is a schematic side view of a fixation device delivery system including another positioning system.

FIG. 48 is a flowchart showing an example method for delivering a transcatheter positioning device to a delivery site in the heart.

FIG. 49 is a flowchart showing an example method for modifying an atrioventricular valve of the heart.

### DETAILED DESCRIPTION

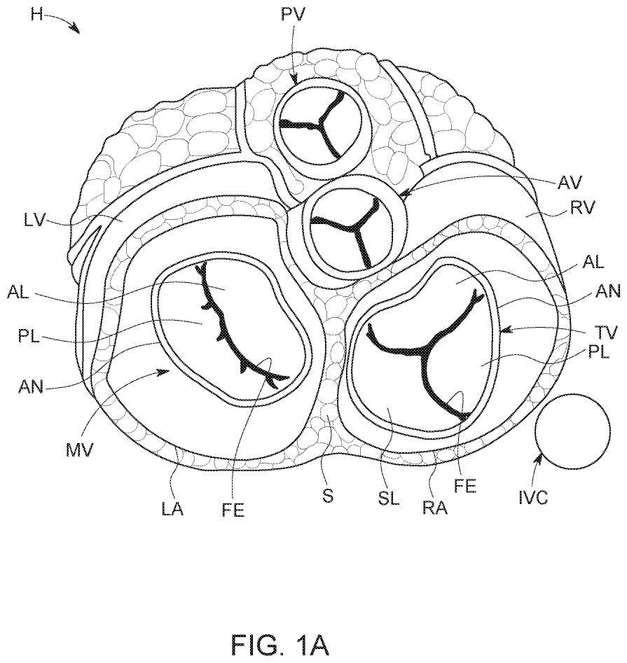

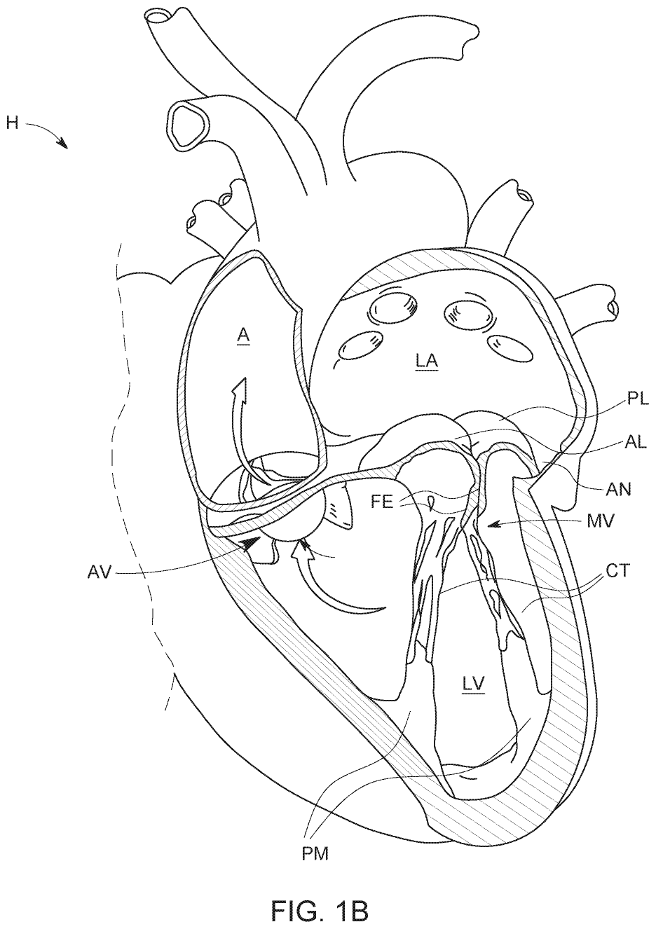

The valves of a heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, the tricuspid valve TV separates the right atrium RA and the right ventricle RV, and the aortic valve AV separates the left ventricle LV and the aorta A. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV typically has three leaflets referred to as the anterior leaflet AL, the posterior leaflet PL, and the septal leaflet SL. However, studies have shown that, although the TV is typically composed of three leaflets of unequal size, in many cases, two or more than three leaflets may be present as anatomic variants in healthy subjects. Thus, reference herein to the tricuspid valve TV should be understood to refer to the atrioventricular valve located between the right atrium RA and right ventricle RV regardless of the number of leaflets be it two, three, or more than three leaflets. However, exemplary embodiments discussed herein refer to the usual anatomic structure of the tricuspid valve TV that includes three leaflets.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

TEER procedures are often performed by obtaining access to the femoral vein, and advancing a fixation device into the right atrium RA via the inferior vena cava IVC, and then crossing the atrial septum S to gain access to the left atrium LA and the mitral valve MV.

As noted above, tricuspid valve regurgitation (“TVR”) is one possible disease state of the tricuspid valve. TVR is a progressive disease and increasing TVR severity is associated with increasing mortality and morbidity. Disease progression is associated with right heart remodeling which includes right ventricle dysfunction and enlargement, increased tricuspid valve coaptation gaps, and progressive tricuspid annulus enlargement. Currently, repair and replacement options take different approaches to reduce regurgitation. Replacement options often have a high screen fail rates due to the large range of annulus sizes and anatomical variability for tricuspid valves as compared to available prosthesis sizes. This anatomical variability may necessitate a large range of device sizes to effectively treat the range of patient anatomies. Tricuspid repair using TEER techniques has proven to be effective in a large proportion of patients with significant TVR. However the progression of disease and lifetime management of such patients remain a challenge.

Tricuspid repair using TEER techniques may be effective in modifying annulus dimensions to prepare the tricuspid valve to receive a transcatheter prosthetic tricuspid valve. Before going into further detail regarding using a TEER procedure in preparation for a transcatheter tricuspid valve replacement (“TTVR”) procedure, examples of TTVR and TEER devices are described below.

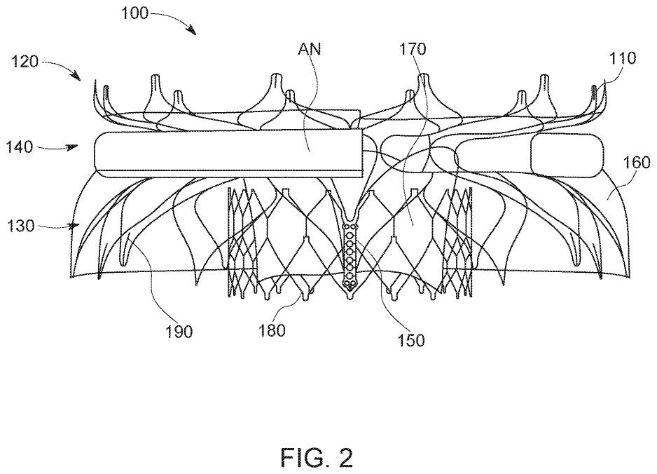

FIG. 2 is a side view of a prosthetic heart valve 100, in particular a prosthetic atrioventricular valve, shown in an expanded, implanted condition. Generally, prosthetic heart valve 100 includes a frame 110, a skirt 160, prosthetic leaflets 170, and a support ring 180. In FIG. 2, the skirt 160 is shown as partially transparent so that internal components of the prosthetic heart valve 100 are visible. Furthermore, a representation of a portion of a native heart valve annulus AN is shown in FIG. 2 with one quarter of the circumference of the valve annulus AN cut-away to better illustrate how a waist of the frame 110 engages the valve annulus AN. The frame 110 may be expandable and collapsible, and formed of a shape-memory material, such as a nickel-titanium alloy such as nitinol. The frame 110 may be shape set (e.g. via heat treatment) to a shape similar to that shown in FIG. 2, for example in which the frame 110 includes a relatively small diameter waist portion 140, with a relatively large diameter atrial anchor or flare 120 and a relatively large diameter ventricular anchor or flare 130 extending outwardly from the waist portion 140. The frame may include a plurality of commissure attachment features 150 extending in an outflow direction from the waist portion 140, and the prosthetic leaflets 170 may be mounted to the frame 110 via the commissure attachment features 150. The support ring 180, if included, may help the prosthetic leaflets 170 and the commissure attachment features 150 maintain a generally cylindrical shape when in use. In some examples, the prosthetic heart valve 100 may anchor, at least in part, by the atrial anchor 120 and ventricular anchor 130 being positioned on opposite sides of the native valve annulus AN. However, tricuspid valve frequently do not have a well-defined annulus, and the annulus may be less fibrous than the annulus of the mitral valve, and thus the native annulus AN may not provide sufficient structure for sufficient anchoring of the prosthetic heart valve 100. Thus, in some examples, the frame 110 may include one or more frictional engagement elements such as tines 190 configured to grip native tissue to assist with anchoring. Still further, in some examples, the skirt 160 may tend to billow outwardly during ventricular systole, which may further anchor the prosthetic heart valve 100 within the native valve annulus AN. It should be understood that prosthetic heart valve 100 is merely one example of a transcatheter prosthetic atrioventricular valve, and various other designs, including those that incorporate elements specific to the mitral valve or elements specific to the tricuspid valve, may be suitable for use in the transcatheter prosthetic valve replacement procedures described in greater detail below. Examples of transcatheter prosthetic atrioventricular valves that show various design options for these types of valves are described in more detail in U.S. Patent Application Publication Nos. 2022/0331097, 2019/0321171, 2024/0325142, and 2019/0142585, the disclosures of which are hereby incorporated by reference herein.

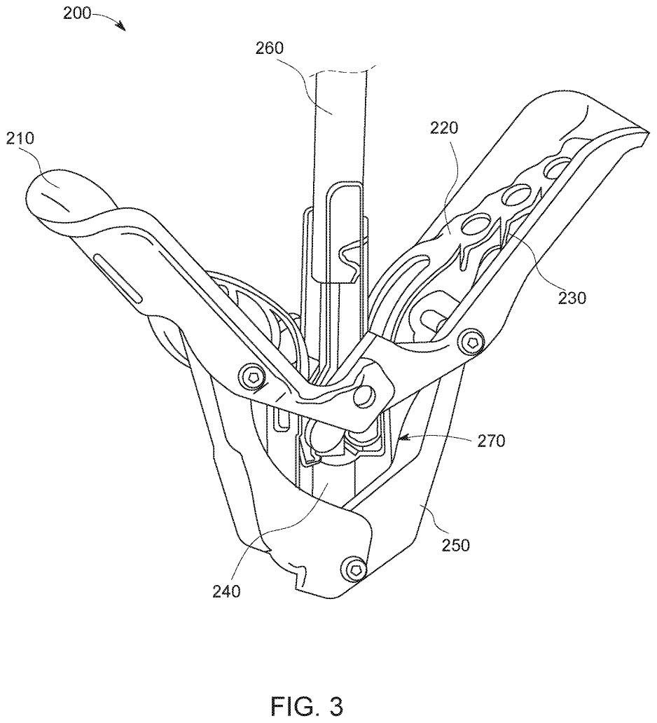

An example fixation device 200 is shown in FIG. 3. Generally, fixation device 200 may include distal elements 210 that include elongate arms. Fixation device 200 may also include a pair of proximal elements 220, which may each have one or more frictional elements 230 extending therefrom. In use, the distal elements 210 may be positioned on outflow surfaces of a pair of native heart valve leaflets, and the proximal elements 230 may be positioned on inflow surfaces of the pair of native heart valve leaflets, with the leaflets being clipped or sandwiched or otherwise secured between the elements to achieve edge-to-edge repair of the native leaflets. Movement of the distal elements 210 may be achieved by movement of a stud 240. Stud 240 may be attached to legs 250 via a rivet or other joint, while opposite ends of the legs 250 may be attached to the distal elements 210 via additional rivets or joints. With this configuration, movement of a delivery system shaft 260 distally will tend to close the distal elements 210, while movement of the delivery system shaft 260 proximally will tend to open the distal elements 210. The fixation device 200 may include a locking mechanism 270 to lock the distal elements at a particular desired closing angle. It should be understood that fixation device 200 is merely one example of a TEER fixation device, and various other designs, including those that incorporate elements specific to the mitral valve or elements specific to the tricuspid valve, may be suitable for use in the TEER procedures described in greater detail below. Additional details of examples of fixation device 200 are provided in U.S. Patent Application Publication No. 2024/0382308, the disclosure of which is hereby incorporated by reference herein.

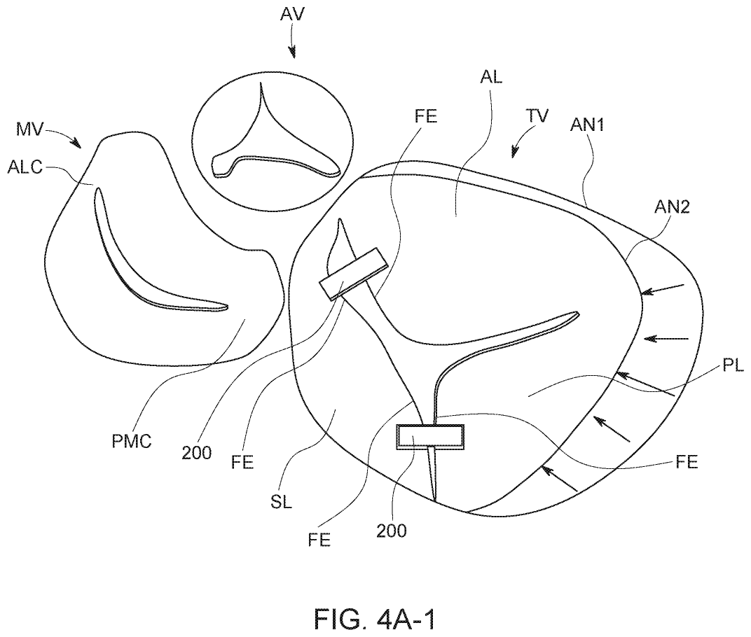

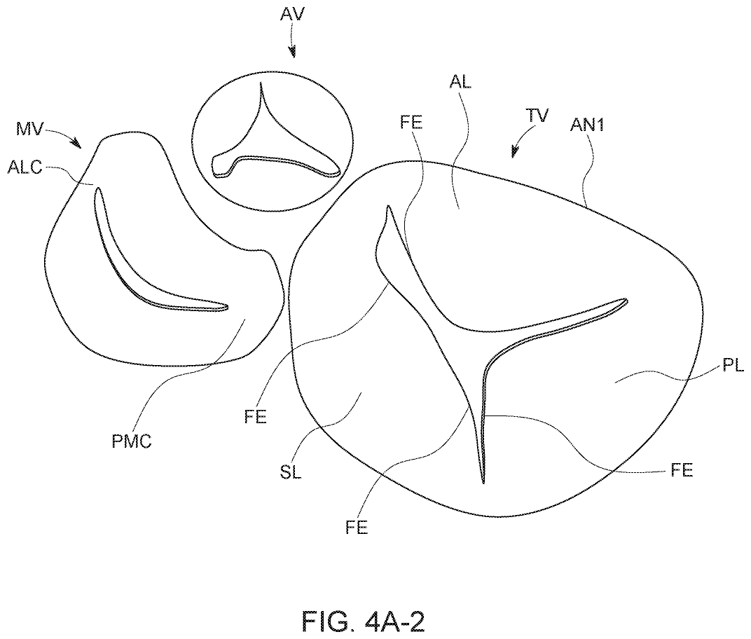

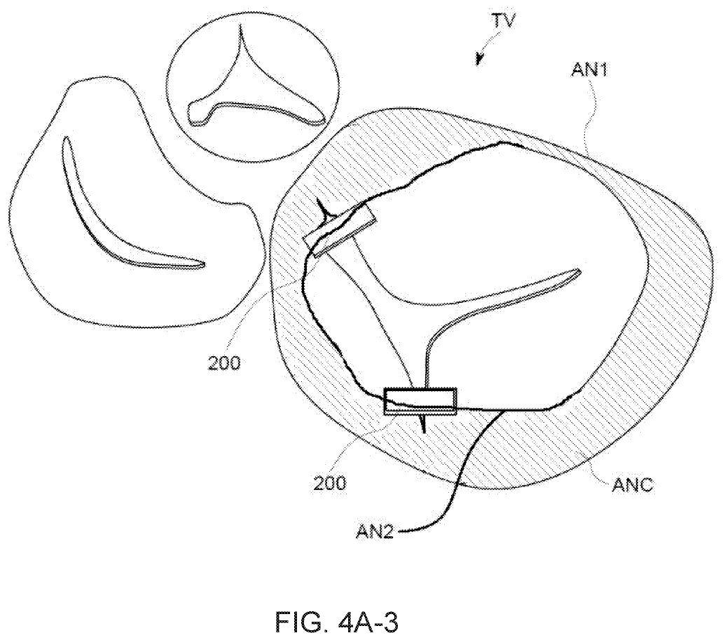

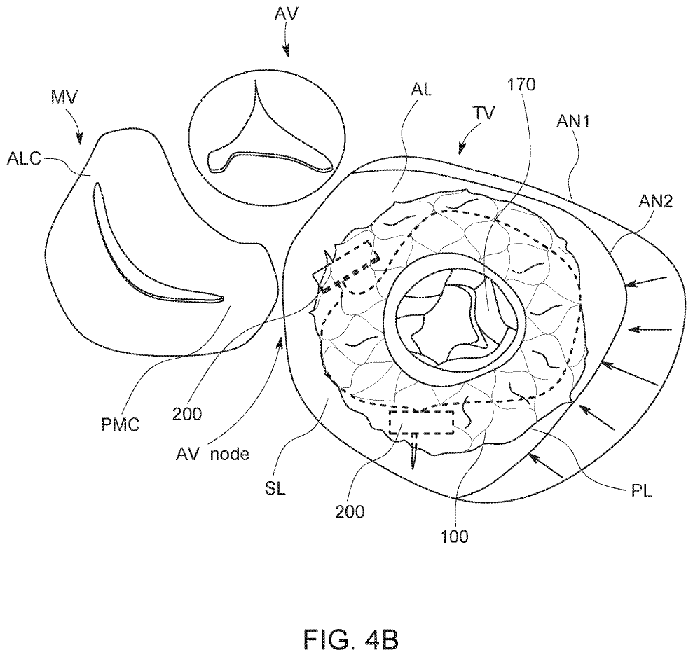

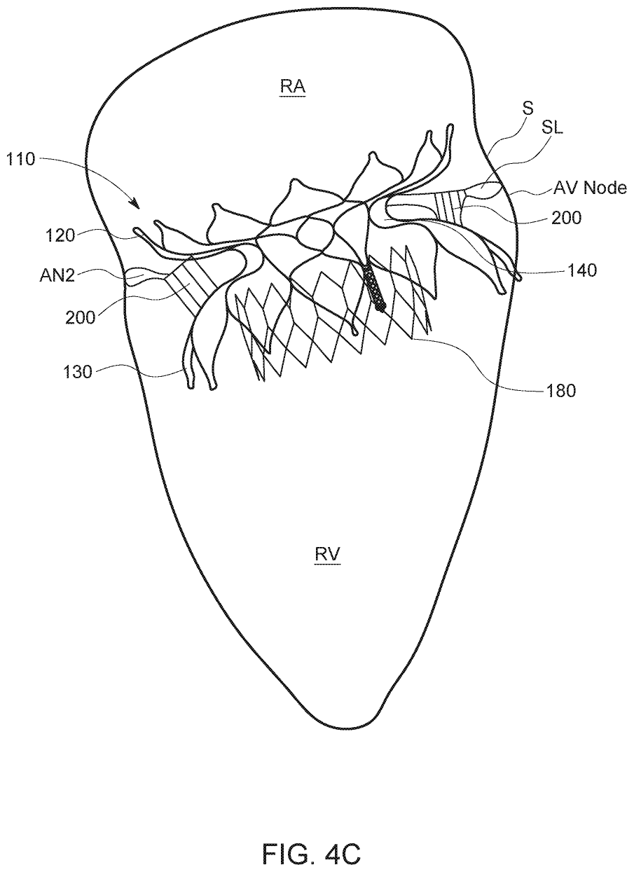

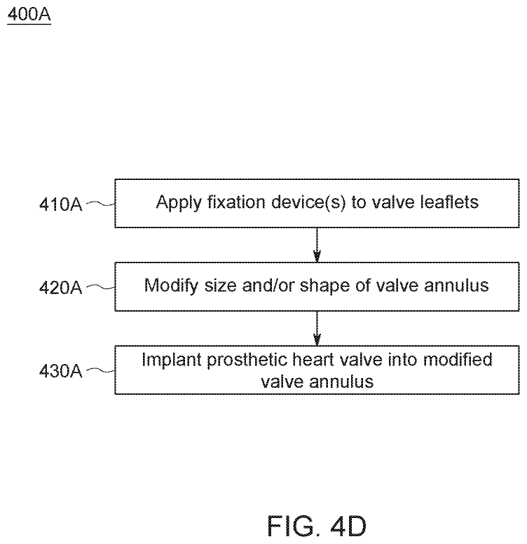

As noted above, tricuspid repair using TEER techniques may be effective in modifying annulus dimensions to prepare the tricuspid valve to receive a transcatheter prosthetic tricuspid valve. For example, FIG. 4A-1 is a schematic representation of an en face view of a heart, including the tricuspid valve TV, mitral valve VM, and aortic valve AV in which a first fixation device 200 has been deployed to engage and approximate free edges FE of the anterior leaflet AL and septal leaflet SL of the tricuspid valve TV, and a second fixation device 200 has been deployed to engage and approximate free edges FE of the posterior leaflet PL and septal leaflet SL of the tricuspid valve TV. FIG. 4A-1 also provides a representation of an original position of the tricuspid valve annulus AN1, which represents the shape and position of the valve annulus AN1 prior to application of the fixation devices 200, as well as a modified shape and position of the valve annulus AN2 after application of the fixation devices 200. FIG. 4A-2 is a schematic en face view showing the view of FIG. 4A-1 prior to the application of the fixation devices to the leaflets, better illustrating the original position of the valve annulus AN1 prior modification and/or re-sizing of the annulus. In the example shown in FIG. 4A-1, following deployment of the two illustrated fixation devices 200, the valve annulus AN2 pulls toward the septum S (the pulling action being represented in FIG. 4A-1 by arrows between the original position of the valve annulus AN1 and the subsequent position of the valve annulus AN2), resulting in a smaller overall area of the valve annulus AN2 and/or movement of the valve annulus AN2 in the direction of the septum S. FIG. 4A-3 is a schematic en face view that illustrates the change in the valve annulus ANC between the original position of the valve annulus AN1 and the subsequent position of the valve annulus AN2. In some examples, prior to modification of the valve annulus AN1, the valve annulus AN1 has an interior perimeter and/or area, which may be measured where the bases of the leaflets meet the annulus, which is larger than the perimeter and/or area of the valve annulus AN2 after being modified, which again may be measured where the bases of the leaflets meat the annulus, with the two perimeter and/or area measurements being taken at the same point in the phase of the cardiac cycle. It should be understood that the fixation devices 200 are schematically represented by rectangles in FIGS. 4A-1 and FIG. 4A-3, and in some examples the portion of fixation devices 200 visible in FIG. 4A-1 and FIG. 4A-3 represent proximal elements 220. However, it should be understood that fixation devices 200 may take other suitable forms, including other types of leaflet fixation devices or clips that are different than the specific, non-limiting example of fixation device 200 shown and described in connection with FIG. 3. Further, although FIG. 4A-1 shows two fixation devices 200, with one engaging and approximating the free edges of the anterior leaflet AL and septal leaflet SL, and the other engaging and approximating the free edges of the posterior leaflet PL and septal leaflet SL, it should be understood that fixation devices 200 (or other types of fixation devices) may be used in different numbers and positions than shown in FIG. 4A-1 to achieve a modified shape and/or position of the valve annulus AN2. For example, only a single fixation device 200 may be used in some examples, and in other examples, two (or more) fixation devices 200 may be applied to the same leaflet pair. Furthermore, in some examples, the modification to the annulus may be achieved immediately after the application of the one or more fixation devices 200 to the leaflets. In other examples, the modification to the annulus may be achieved over a longer duration period, such as weeks, months, or years. For example, although some amount of modification may be expected shortly after application of the fixation device(s) 200, further modification may occur after the heart responds (e.g. by remodeling) over time as regurgitation is reduced or eliminated.

Now referring in addition to FIGS. 4B-4C, FIG. 4B is a representation of the en face view of the heart of FIG. 4A-1, and FIG. 4C is a representation of a lateral view of the heart of FIG. 4A-1, after transcatheter prosthetic heart valve 100 has been implanted in the tricuspid valve TV. It should be understood that, in FIG. 4B, the top or atrial side of the prosthetic heart valve 100 is shown as partially transparent to show examples of positions of the fixation devices 200 and the leaflets SL, PL, AL that may otherwise not be visible. It should also be understood that, in FIG. 4C, only the frame 110 and support ring 180 of prosthetic heart valve 100 are illustrated, with all “soft” components of the prosthetic heart valve 100, such as the prosthetic leaflets 170 and skirt 160, omitted from the view to better illustrate the position of the prosthetic heart valve 100. If an attempt were made to implant prosthetic heart valve 100 in the tricuspid valve TV prior to the application of fixation devices 200, and prior to modification and/or resizing of the valve annulus AN1, the prosthetic heart valve 100 may not fit because of the large size of the valve annulus AN1 pre-modification. Thus, at least one benefit of modification and/or resizing of the tricuspid valve TV with fixation devices 200 to reduce the size of a highly dilated valve annulus AN1 is that, after the annulus is modified to be a smaller sized and/or shaped valve annulus AN2, the valve annulus AN2 may be more likely to be sized to receive a transcatheter prosthetic heart valve 100. As noted above, prosthetic heart valves are typically manufactured in a limited number of sizes, and a highly dilated valve annulus AN1 may be too large to receive an existing prosthetic heart valve 100, such that modifying the annulus to a smaller size and/or shape may allow an existing prosthetic heart valve 100 to be implanted in the modified valve annulus AN2. Stated in another way, a more limited range of sizes of prosthetic heart valves 100 (or variants thereof) may be able to treat a larger patient population if fixation devices 200 (or variants thereof) are used to modify and/or reduce the size and/or shape of the native valve annulus AN1 of patients. In other words, if the prosthetic heart valve is self-expandable, it may have an operational size in the absence of applied forced, where the operational size would be too small to anchor to the tricuspid valve TV and/or the valve annulus AN when the valve annulus has the dilated size AN1, but that same operational size may be sufficiently large to anchor to the tricuspid valve TV and/or the valve annulus AN when the valve annulus has the modified size AN2.

In addition to, or separate from, the potential first benefit addressed above, the existence of the fixation devices 200 may also provide for more stable anchoring of the prosthetic heart valve 100. For example, as best shown in FIG. 4C, after deployment of prosthetic heart valve 100 into the tricuspid valve TV, one or more of the fixation devices 200 may be located near the waist portion 140 of the frame 110, between the atrial anchor 120 and the ventricular anchor 130. In some examples, the ventricular-facing side of the atrial anchor 120 and/or the atrial-facing side of the ventricular anchor 130 may contact (directly, or indirectly if any skirt such as skirt 160 is positioned on the outer surface of the frame 110) one or more of the fixation devices 200. This contact may provide further stability (e.g. better anchoring) of the prosthetic heart valve 100, for example compared to if prosthetic heart valve 100 only contacted tissue of the native valve annulus and/or the leaflets. This enhanced anchoring may be particularly notable when prosthetic heart valve 100 is implanted into a tricuspid valve TV, for example because the annulus AN of a tricuspid valve TV is typically less well-formed and less-rigid (e.g. less fibrous) compared to the annulus AN of a mitral valve MV. Even though the enhanced anchoring benefit described above may be more pronounced in the tricuspid valve TV, it should be understood that the benefit may still be useful in other valves, such as the mitral valve MV. Further, although FIGS. 4B-4C are shown and described in the context of implantation of the prosthetic heart valve 100 shown and described in connection with FIG. 2, other examples of prosthetic heart valves may have similar or identical benefits when being implanted after intentional modification of the native valve annulus using one or more fixation devices such as fixation devices 200.

FIG. 4D is a flowchart showing example steps of a prosthetic heart valve implantation method 400A. In one step 410A of the method 400A, one or more fixation devices are applied to one or more pairs of native valve leaflets. If the valve being replaced is a mitral valve MV, one or more fixation devices may be applied to engage and approximate the free edges FE of the anterior leaflet AL and posterior leaflet PL of the mitral valve MV. If the valve being replaced is a tricuspid valve TV, one or more fixation devices may be applied to engage and approximate (i) the free edges FE of the septal leaflet SL and the anterior leaflet AL, (ii) the free edges FE of the septal leaflet SL and the posterior leaflet PL, or (iii) the free edges FE of the anterior leaflet AL and the posterior leaflet PL. It should be understood that the leaflet pairs described above are exemplary only, particularly given that native mitral and tricuspid valves may have variability in the number of actual leaflets that form the valve (e.g. mitral valves MV do not always exist with a well-defined bicuspid leaflet configuration and tricuspid valves TV do not always exist with a well-defined tricuspid leaflet configuration). The one or more fixation devices applied to the pair(s) of leaflets in step 410A may take the form of fixation device 200 described above in connection with FIG. 3, or may take any other suitable form that provides for leaflet engagement and/or approximation of leaflet free edges FE, for example by pinching, sandwiching, or otherwise clipping the free edges FE of native leaflets together. In exemplary step 420A of method 400A, the size and/or shape of the heart valve annulus is modified by the one or more fixation devices applied in step 410A. The modification may reduce the size and/or shape of the native valve annulus. In some examples, some or all the modification of valve annulus may occur rapidly, for example immediately upon or shortly after application of the fixation devices. If the valve being replaced is the mitral valve MV, it is the native mitral valve annulus that is modified in step 420A, whereas if the valve being replaced is the tricuspid valve TV, it is the native tricuspid valve annulus that is modified in step 420A. After the native valve annulus has been modified, a transcatheter prosthetic heart valve is delivered to the native heart valve being replaced and deployed into the modified valve annulus in step 430A. In some examples, the method 400A may be performed in a single overall procedure, instead of in separate procedures over time. For example, the method 400A may be performed with the patient remaining in a single location (e.g. within the same operating site or the same catheter lab setting) for steps 410A, 420A, and 430A and/or steps 410A, 420A, and 430A may all be performed within the same day. In another example, the method 400A may be performed with the patient remaining under a single continuous round of anesthesia (e.g. while remaining at least partially under the effects of anesthesia). In some examples, the prosthetic heart valve implanted in step 430A may be the prosthetic heart valve 100 shown and described in connection with FIG. 2, although it should be understood that any transcatheter prosthetic mitral or tricuspid valve may be implanted in step 430A. If the prosthetic heart valve implanted in step 430A includes a frame with an atrial anchor or flare and a ventricular anchor or flare, which may be similar to the configuration shown and described in connection with frame 110, one or more of the fixation devices may be positioned between the atrial anchor or flare and the ventricular anchor or flare to provide for additional anchoring stability. However, in other examples, the prosthetic heart valve may not interact substantially, or at all, with the fixation devices applied in step 410A. Although in some examples, the method 400A may be performed as part of a single overall procedure, in other examples, the method 400A may be performed with significant time elapsing between step 410A and step 430A. In such examples, step 410A may be performed, with an example result being reducing severe or torrential regurgitation acutely, but then the patient is left to recover (and/or the patient's heart is allowed to compensate) for a period of time, such as weeks, months, or years. After the heart remodels over this time period (and step 420A occurs), the prosthetic heart valve may then be implanted in step 430A. In the cases in which step 420A occurs over a longer time period (e.g. weeks, months, or years), the fixation device(s) may endothelialize as the surrounding tissue heals over time. In these examples, the prosthetic heart valve implanted in step 430A may be performed (i) without modification of the prior-implanted fixation device(s) 200, (ii) after cutting the leaflet(s) but without removing the fixation device(s) 200 from the leaflets, or the leaflets can be cut on either side of the fixation device(s) so that the fixation device(s) 200 can be removed completely prior to deployment of the replacement valve

In some examples, the prosthetic heart valve implanted in step 430A is a self-expanding prosthetic heart valve. Prosthetic heart valve 100 is one example of a self-expanding prosthetic heart valve. However, in other examples, the prosthetic heart valve implanted in step 430A is a balloon-expandable prosthetic heart valve (e.g. the prosthetic heart valve includes a plastically-expandable frame, which may be formed of stainless steel, cobalt chromium, or other suitable metals or metal alloys). Balloon-expandable prosthetic heart valves often have different anchoring considerations than self-expandable prosthetic heart valves. For example, balloon-expandable prosthetic aortic valves often rely on calcification of the aortic valve to provide a surface against which the prosthetic heart valve is able to anchor upon expansion. Some balloon-expandable prosthetic mitral valves require a separate anchor or docking device to be implanted prior to balloon-expansion of the prosthetic mitral valve to allow for sufficiently rigid structure against which the prosthetic mitral valve may anchor. One example of such a balloon-expandable prosthetic heart valve that employs docking element into which the balloon-expandable prosthetic heart valve is anchored is described in greater detail in U.S. Patent Application Publication No. 2018/0177594, the disclosure of which is hereby incorporated by reference herein. In some examples of method 400A, in step 410A, at least two fixation devices may be applied on opposite sides of the native valve opening. For example, if the valve being replaced is a mitral valve MV, at least one fixation device may be applied to the anterior leaflet AL and posterior leaflet PL near the anterolateral commissure ALC, and at least one fixation device may be applied to the anterior leaflet AL and the posterior leaflet PL near the posteromedial commissure PMC. Then, in step 430A, the prosthetic heart valve may be balloon-expanded in the mitral valve MV so that the fixation devices are on generally opposite sides of the prosthetic heart valve, with the fixation devices providing enough rigidity for the balloon-expandable prosthetic heart valve to anchor against (e.g. to resist migration into the left atrium during use). If the valve being replaced is a tricuspid valve TV, at least two fixation devices may be applied to the tricuspid valve TV to engage and approximate the free edges FE of two different pairs of leaflets, or otherwise at least three fixation devices may be applied to the tricuspid valve TV to engage and approximate the free edges FE of all three pairs of leaflets, such that each fixation device is near the commissure that connects the two leaflets of the leaflet pair. Then, in step 430A, the prosthetic heart valve may be balloon-expanded in the tricuspid valve TV so that the prosthetic heart valve contacts two or three of the fixation devices. For example, if three fixation devices are used, the fixation devices may be generally spaced at about 120 degree intervals (although the exact spacing would be dependent, at least in part, on the specific configuration of the native anatomy) so that the fixation devices provide three rigid anchor points to anchor the prosthetic heart valve in place (e.g. to resist migration into the right atrium during use). If only two fixation devices are used, the relative positioning of the two fixation devices may still be able to provide sufficient rigidity against which the balloon-expandable prosthetic heart valve may anchor. It should be understood that, if fixation devices applied in step 410A are being used primarily for providing rigidity for anchoring a balloon-expandable prosthetic heart valve, method 400A may be completed with or without any (or any substantial) modification in the size or geometry of the native valve annulus as in step 420A, and steps 410A and 430A may be performed as part of a single overall procedure, or as completely separate procedures (e.g. the balloon-expandable prosthetic heart valve could be implanted days, weeks, months, or years after the fixation devices are applied). It should also be understood that, although examples are described in this paragraph in which at least two fixation devices are applied to provide rigidity for anchoring a balloon-expandable prosthetic heart valve, even a single fixation device may provide sufficient rigidity for a balloon-expandable prosthetic heart valve, with the native tissue (whether or not the single fixation device significantly modifies the shape of the annulus) providing sufficient anchoring structure in combination with the single fixation device.

Although method 400A is generally described in connection with using one or more fixation devices to (i) modify the size and/or shape of the native annulus to make the native annulus more suited to receive an available size of an existing prosthetic heart valve and/or to (ii) provide additional anchor points for a self-expanding or balloon-expandable prosthetic heart valve to anchor against, in some examples one or more fixation devices may be used to assist with positioning the prosthetic heart valve in a desirable position and/or orientation relative to other portions of the native anatomy.

One example of using one or more fixation devices to allow for preferential positioning of a prosthetic heart valve involves avoidance of contact with the atrioventricular (“AV”) node. The AV node is an anatomical structure (or cluster of structures) that assists with electrical conduction in the heart to coordinate beating of the heart. The AV node is typically located in the right atrium RA, on or near the atrial septum S adjacent the septal leaflet SL of the tricuspid valve TV. If a prosthetic tricuspid valve is implanted in a way that results in structure of the prosthetic tricuspid valve pressing against, or otherwise contacting the AV node (a representative position of which is illustrated in FIGS. 4B-4C), the proper conduction of electricity via the AV node may be disturbed, potentially resulting in irregular beating of the heart which may need to be treated by implantation of a pacemaker.

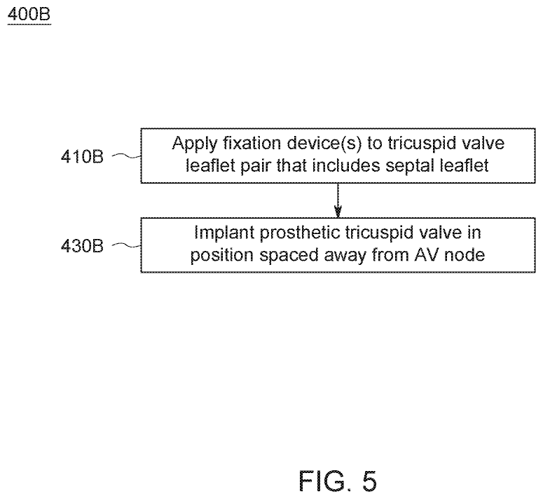

FIG. 5 is a flowchart showing example steps of a prosthetic heart valve implantation method 400B. Although method 400B may be generally similar to method 400A, method 400B is specific to preparing the tricuspid valve TV for receiving a prosthetic tricuspid valve in a position and/or orientation such that the prosthetic tricuspid valve does not contact the AV node. In one step 410B of the method 400B, at least one fixation device is applied to a pair of leaflets that includes the septal leaflet SL, including either (i) the septal leaflet SL and the anterior leaflet AL and/or (ii) the septal leaflet SL and the posterior leaflet PL. One or more fixation devices may be applied to either, or both, leaflet pairs. The illustration shown in FIG. 4B shows one example in which a first fixation device is applied to (i) the septal leaflet SL and the anterior leaflet AL pair, while a second fixation device is applied to (ii) the septal leaflet SL and the posterior leaflet PL pair. Performing step 410B may result in modification of the size and/or shape of the valve annulus as in step 420A of method 400A, although it should be understood that such a step is not necessarily required as part of method 400B. In some examples, to be able to position the prosthetic heart valve even farther from the AV node, two, or more than two, fixation devices may be applied to (i) the septal leaflet SL and the anterior leaflet AL pair, and/or (ii) the septal leaflet SL and the posterior leaflet PL pair, with more fixation devices generally corresponding to being able to position the prosthetic tricuspid valve farther from the AV node. In some examples, zero fixation devices may be applied to the anterior leaflet AL and posterior leaflet PL pair, as such fixation devices may tend to cause a prosthetic heart valve implanted into the tricuspid valve TV to be positioned closer to the AV node. However, in other examples, one or more fixation devices may still be applied to the anterior leaflet AL and posterior leaflet PL pair if the other fixation devices allow for the prosthetic tricuspid valve to be deployed in a position and/or orientation out of contact with the AV node. The one or more fixation devices applied to the pair(s) of leaflets in step 410B may take the form of fixation device 200 described above in connection with FIG. 3, or may take any other suitable form that provides for leaflet engagement and/or approximation of free edges FE of leaflets, for example by pinching, sandwiching, or otherwise clipping the free edges FE of native leaflets together. After the fixation device(s) have been applied in step 410B, a transcatheter prosthetic tricuspid valve is delivered to the tricuspid valve TV and deployed in step 430B so that no portions of the prosthetic tricuspid valve contact the AV node. In some examples, the method 400B may be performed in a single overall procedure, instead of in separate procedures over time. For example, the method 400B may be performed with the patient remaining in a single location (e.g. within the same operating site or the same catheter lab setting) for steps 410B and 430B and/or steps 410B and 430B all being performed within the same day. In some examples, the prosthetic tricuspid valve implanted in step 430B may be the prosthetic heart valve 100 shown and described in connection with FIG. 2, although it should be understood that any transcatheter prosthetic tricuspid valve may be implanted in step 430B. Further, it should be understood that although a primary goal of method 400B is to implant a prosthetic tricuspid valve so that it avoids disturbing electrical conduction within the heart, other benefits, including any of those described in connection with method 400A, may also be realized by method 400B.

Another example of using one or more fixation devices to allow for preferential positioning of a prosthetic heart valve involves avoidance of obstruction of the LVOT by the prosthetic heart valve. Although the above-described modification of the native valve annulus (e.g. as described in connection with step 420A of method 400A) may be performed on either the tricuspid valve TV or the mitral valve MV, the range of annulus sizes for the mitral valve among patients is typically not as variable as seen for the tricuspid valve. One issue that is particularly relevant for the mitral valve, however, is possible obstruction of the LVOT from a prosthetic mitral valve. The LVOT is the path by which blood flows from the left ventricle LV during ventricular systole through the open aortic valve AV toward the aorta A. Structures of prosthetic mitral valves that extend any distance into the left ventricle LV may be at risk of obstructing blood flow along the LVOT, and as noted above, such obstruction is one reason that a patient may be excluded from a prosthetic mitral valve replacement procedure. The likelihood of such obstruction may be a factor of one or both of (i) the distance into the left ventricle LV which structure of the prosthetic mitral valve extends and (ii) the distance between the LVOT and any structures of the prosthetic mitral valve extending into the left ventricle LV. A similar technique as described in connection with method 400B may be employed in a patient's mitral valve to establish or increase an amount of offset between the LVOT and structures of the prosthetic mitral valve extending into the left ventricle LV.

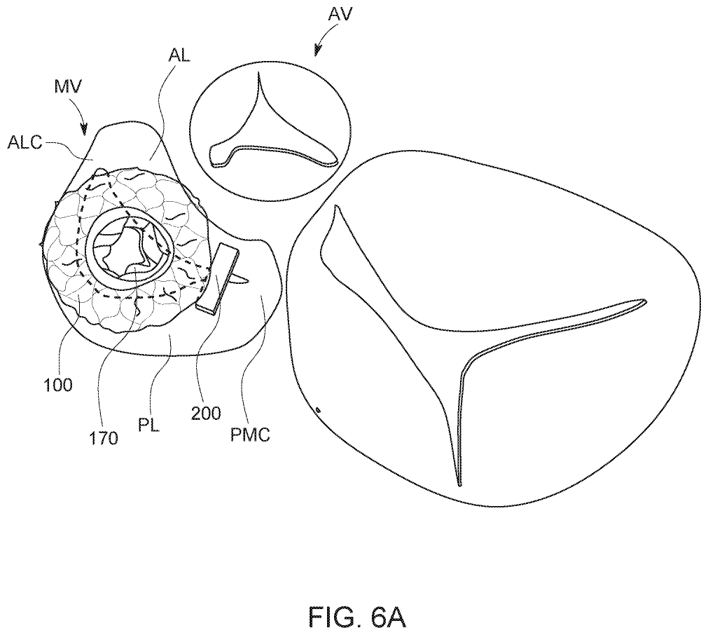

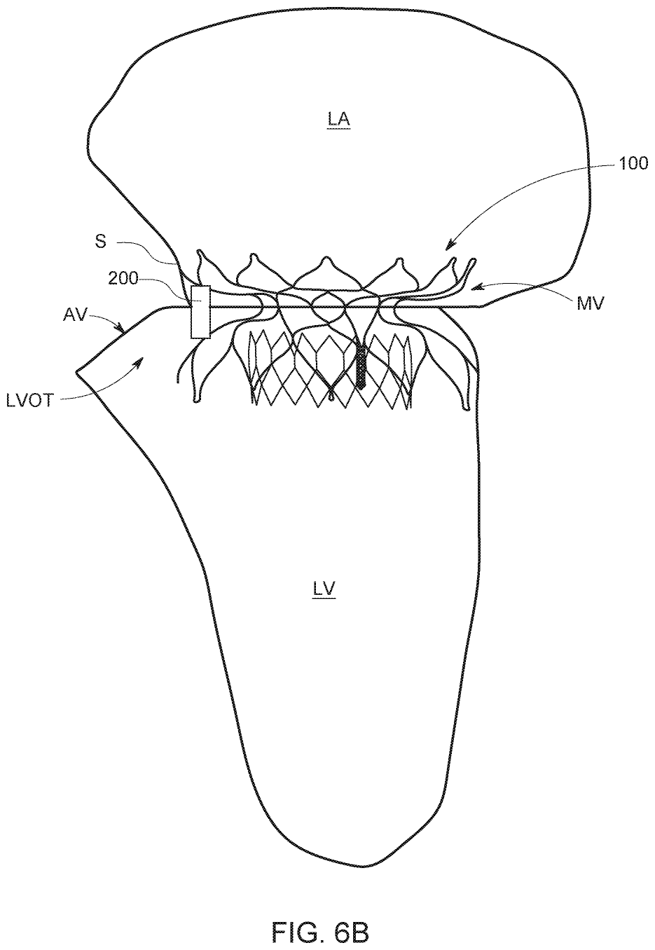

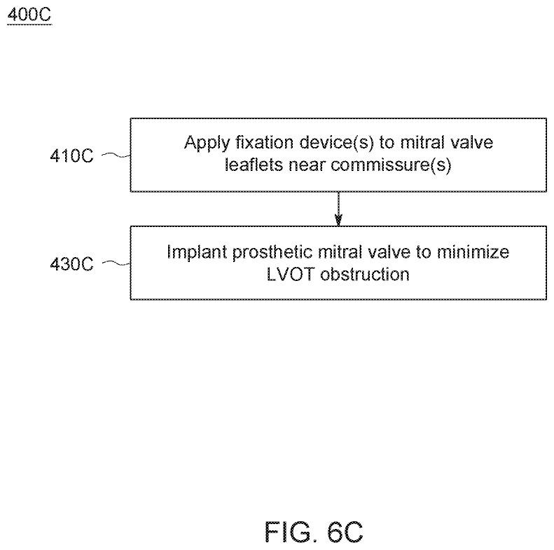

FIG. 6A is a representation of an en face view of the heart (similar to FIGS. 4A-1 through FIG. 4B) after a fixation device has been applied to the mitral valve MV and a prosthetic mitral valve has been implanted into the mitral valve MV. FIG. 6B is a representation of a lateral view of the heart of FIG. 6A, after the prosthetic mitral valve has been implanted in the mitral valve MV. It should be understood that, in FIG. 6A, the top or atrial side of the prosthetic mitral valve is shown as partially transparent to show examples of the position of the fixation device 200 and the leaflets AL, PL, that may otherwise not be clearly visible. It should also be understood that, in FIG. 6B, only the frame 110 and support ring 180 of prosthetic heart valve 100 are illustrated, with all “soft” components of the prosthetic heart valve 100, such as the prosthetic leaflets 170 and skirt 160, omitted from the view to better illustrate the position of the prosthetic heart valve 100.

FIG. 6C is a flowchart showing example steps of a prosthetic heart valve implantation method 400C. Although method 400C may be generally similar to method 400A, method 400C is specific to preparing the mitral valve MV for receiving a prosthetic mitral valve in a position and/or orientation such that the LVOT obstruction is minimized or avoided. In one step 410C of the method 400C, at least one fixation device is applied to the anterior leaflet AL and posterior leaflet PL, in a position near the one of the commissures (e.g. the anterolateral commissure AMC or posteromedial commissure PMC) of the mitral valve MV. In the example shown in FIG. 6A, a fixation device is applied to the anterior leaflet AL and posterior leaflet PL pair near the posteromedial commissure PMC, although in other examples, the fixation device may be applied to the leaflet pair near the anterolateral commissure ALC. In some examples, two or more fixation devices may be applied to the leaflet pair near the posteromedial commissure PMC, and in other examples, two or more fixation devices may be applied to the leaflet pair near the anterolateral commissure ALC. In some examples, two or more fixation devices may be applied to the leaflet pair near the posteromedial commissure PMC and also near the anterolateral commissure ALC. Performing step 410C may result in modification of the size and/or shape of the valve annulus as in step 420A of method 400A, although it should be understood that such a step is not necessarily required as part of method 400C. The one or more fixation devices applied to the pair(s) of leaflets in step 410C may take the form of fixation device 200 described above in connection with FIG. 3, or may take any other suitable form that provides for leaflet engagement and/or approximation of free edges FE of leaflets, for example by pinching, sandwiching, or otherwise clipping the free edges FE of native leaflets together. After the fixation device(s) have been applied in step 410C, a transcatheter prosthetic mitral valve is delivered to the mitral valve MV and deployed in step 430C so that any obstruction of the LVOT by structures of the prosthetic mitral valve is minimized or eliminated. In some examples, the method 400C may be performed in a single overall procedure, instead of in separate procedures over time. For example, the method 400C may be performed with the patient remaining in a single location (e.g. within the same operating site or the same catheter lab setting) for steps 410C and 430C and/or steps 410C and 430C all being performed within the same day. In some examples, the prosthetic mitral valve implanted in step 430C may be the prosthetic heart valve 100 shown and described in connection with FIG. 2, although it should be understood that any transcatheter prosthetic mitral valve may be implanted in step 430C. Upon implantation, as illustrated for example in FIG. 6B, the prosthetic mitral valve may be positioned more posteriorly (and thus more offset from the LVOT) due to the existence and position of the fixation device(s) than would otherwise be seen in the absence of the fixation device(s). Further, it should be understood that although a primary goal of method 400C is to implant a prosthetic mitral valve so that obstruction of the LVOT is minimized or eliminated, other benefits, including any of those described in connection with method 400A, may also be realized by method 400C.

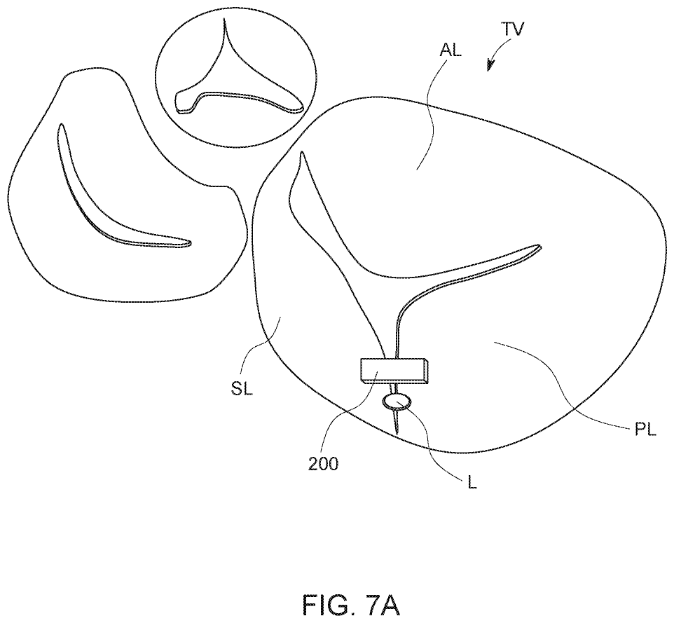

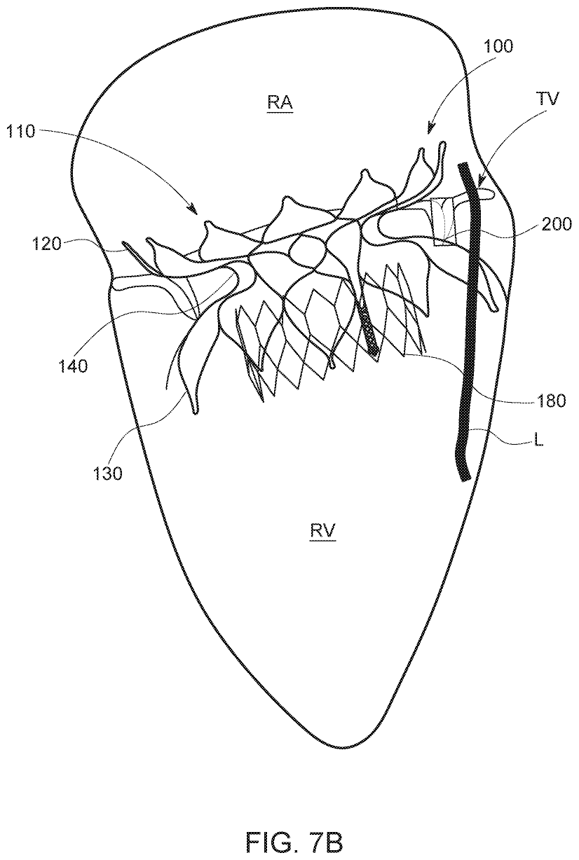

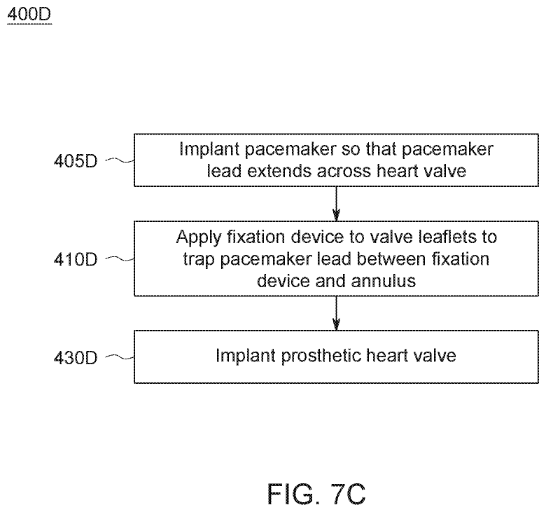

Another consideration with transcatheter prosthetic atrioventricular valve implantations is possible interference from pacemaker leads. When a pacemaker is implanted into a patient, there are typically two lead wires, one of which passes from the superior vena cava, into the right atrium RA, through the tricuspid valve TV, and into the right ventricle RV where an end of the lead is engaged with heart tissue of the left ventricle LV. Because this lead placement is typical, the examples shown and described in connection with FIGS. 7A-7C focus on situations in which a pacemaker lead passes through the tricuspid valve TV. However, it should be understood that the methods described in connection with FIGS. 7A-7C could similarly be applied to situations in which a pacemaker lead passes through the mitral valve MV. Whether the pacemaker lead passes through the tricuspid valve TV or mitral valve MV, there is an overlap between patients who have a pacemaker that end up needing a replacement heart valve, as well as an overlap between patients who have a replacement heart valve that end up needing a pacemaker. In fact, at least some patients may require a replacement heart valve as a direct result of regurgitation occurring at the heart valve due to the pacemaker lead making proper coaptation of the native leaflets impossible. Similarly, at least some patients may require a pacemaker as a direct result of electrical conduction disturbances resulting from a prosthetic heart valve implantation (e.g. because a prosthetic heart valve is pressing against the AV node). In situations in which a patient that has (or needs) a pacemaker with a lead crossing the tricuspid valve TV is also a candidate for a transcatheter prosthetic tricuspid valve implantation, one or more fixation devices may be used to manage the pacemaker lead to help ensure it does not interfere with the implantation and/or functioning of the prosthetic tricuspid valve.

FIG. 7A is a representation of an en face view of the heart (similar to FIG. 4A-1) after a fixation device has been applied to the tricuspid valve TV, and FIG. 7B is a representation of a lateral view of the heart of FIG. 7A, after a prosthetic tricuspid valve has been implanted in the tricuspid valve TV. It should also be understood that, in FIG. 7B, only the frame 110 and support ring 180 of prosthetic heart valve 100 are illustrated, with all “soft” components of the prosthetic heart valve 100, such as the prosthetic leaflets 170 and skirt 160, omitted from the view to better illustrate the position of the prosthetic heart valve 100.

FIG. 7C is a flowchart showing example steps of a prosthetic heart valve implantation method 400D. In one step 405D of method 400D, a pacemaker is implanted so that a pacemaker lead L extends through a native heart valve (including a tricuspid valve TV or a mitral valve MV). It should be understood that, in some examples of method 400D, the pacemaker is already implanted prior to a determination being made to implant a prosthetic heart valve. In this scenario, step 405D may be performed days, weeks, months, or years prior to the remaining steps of method 400D. In other examples, step 405D may be performed with the other steps of method 400D as part of a single overall procedure, for example to either (i) treat a conduction issue that needs to be treated or (ii) to pre-emptively address possible conduction issues that might result from the prosthetic heart valve implantation. Regardless of the timing of step 405D, in step 410D of the method 400D, at least one fixation device is applied to a pair of tricuspid valve leaflets TV. The particular leaflet pair to which the fixation device is applied may be based, at least in part, on where the pacemaker lead is positioned. For example, if the pacemaker lead L is generally positioned near the commissure between the septal leaflet SL and the posterior leaflet PL, the fixation device may be applied to that leaflet pair so that the pacemaker lead L becomes trapped or “jailed” between the fixation device and the native valve annulus. As used herein, the terms “trapping” and “jailing” are used interchangeably. These terms, when used in connection with trapping or jailing a pacemaker lead, refer to the pacemaker lead being circumferentially surrounded by or bounded by a combination of patient tissue and one or more object(s) implanted into or onto patient tissue. In other words, upon implanting one or more object(s) (e.g. a fixation device) into or onto patient tissue to trap or jail the pacemaker lead, the pacemaker lead is restricted from radial movement (e.g. in a direction orthogonal to blood flow) beyond a boundary defined by the patient tissue and the object(s) implanted into or onto patient tissue. If the pacemaker lead is generally positioned closer to a different commissure, the fixation device may be applied to the relevant leaflet pair defining that commissure to trap the pacemaker lead between the fixation device and the native valve annulus. Performing step 410D may result in modification of the size and/or shape of the valve annulus as in step 420A of method 400A, although it should be understood that such a step is not necessarily required as part of method 400D. Further, method 400D may be combined with method 400B to help ensure the prosthetic heart valve avoids contact with the AV node, although such contact may be less detrimental if a pacemaker is already functioning to maintain a desired heart rhythm. The one or more fixation devices applied to the pair(s) of leaflets in step 410D may take the form of fixation device 200 described above in connection with FIG. 3, or may take any other suitable form that provides for leaflet engagement and/or approximation of free edges FE of leaflets, for example by pinching, sandwiching, or otherwise clipping the free edges FE of native leaflets together in a way that jails or otherwise restricts movement of the pacemaker lead L. After the fixation device(s) have been applied in step 410D, a transcatheter prosthetic tricuspid valve is delivered to the tricuspid valve TV and deployed in step 430D. Because the pacemaker lead L has already been jailed by the fixation device 200, the position of the pacemaker lead L is known and any significant movement of the pacemaker lead L that could otherwise interfere with the implantation and/or operation of the prosthetic heart valve 100 is mitigated. For example, as shown in the example of FIG. 7B (which omits the superior portions of the pacemaker lead L which would typically extend upward and into the superior vena cava), the portion of the pacemaker lead L traversing the tricuspid valve TV may be held tightly against the annulus of the tricuspid valve TV by the fixation device 200 so that it is largely out of the way and prevented from interfering with the deployment and/or operation of the prosthetic tricuspid valve 100. As noted above, in some examples, all steps of the method 400D may be performed in a single overall procedure, instead of in separate procedures over time. For example, the method 400D may be performed with the patient remaining in a single location (e.g. within the same operating site or the same catheter lab setting) for steps 405D, 410D and 430D and/or steps 405D, 410D and 430D may all be performed within the same day. However, the various steps may be performed as parts of different procedures over a longer time period. In some examples, the prosthetic tricuspid valve implanted in step 430D may be the prosthetic heart valve 100 shown and described in connection with FIG. 2, although it should be understood that any transcatheter prosthetic mitral valve may be implanted in step 430D. Further, it should be understood that although a primary goal of method 400D is to jail or otherwise restrict movement of a pacemaker lead L in preparation for implantation of a prosthetic tricuspid valve, other benefits, including any of those described in connection with method 400A, may also be realized by method 400D. Further, if method 400D is being applied to a mitral valve, method 400D may be combined with steps of method 400C, and if method 400D is being applied to a tricuspid valve, method 400D may be combined with steps of method 400B.

Although method 400D is described in the context of using a fixation device to jail a previously implanted pacemaker lead L, in some examples, the order may be reversed. In other words, if a patient with a fixation device requires a pacemaker lead to be implanted, space between the fixation device and the adjacent leaflet commissure may be used as a “port” through which the pacemaker lead L may be inserted. In this example, a replacement heart valve need not be implanted (although it may be), with one benefit being the existence of a pre-created “port” through which the pacemaker lead L may be passed and which jails the pacemaker lead L as described above.

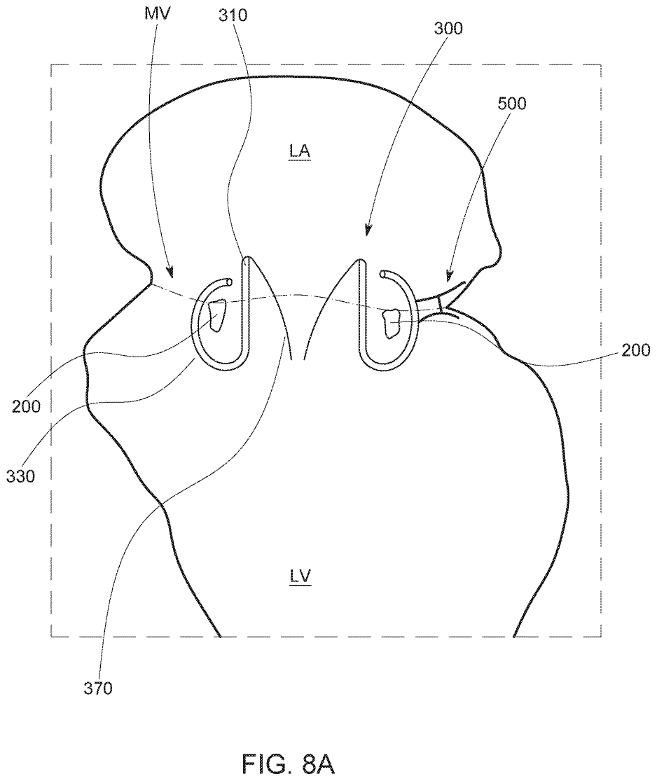

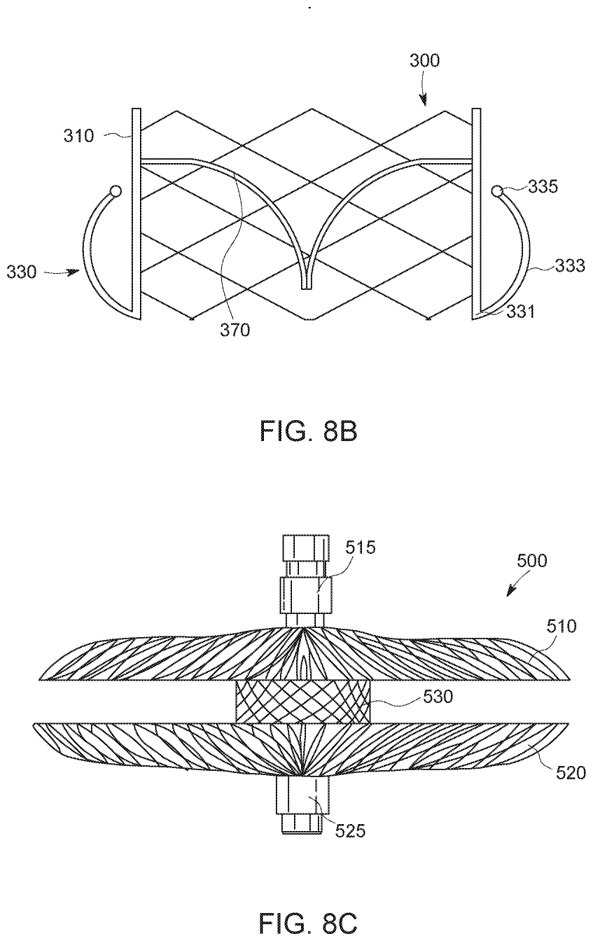

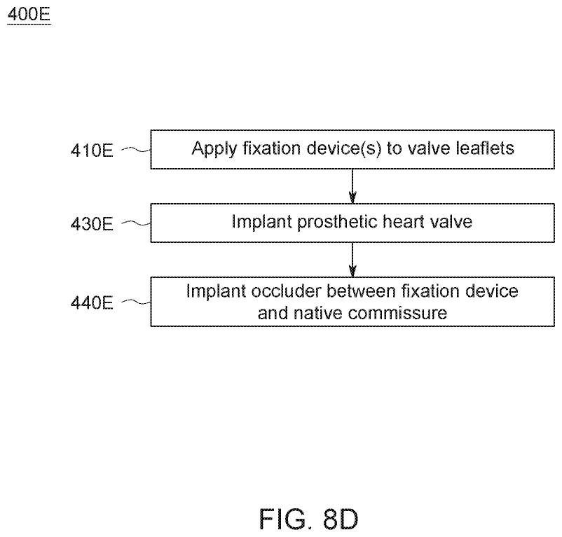

In some examples, a prosthetic heart valve have features designed specifically to interact in a particular fashion with fixation devices that are applied to leaflets of a mitral valve MV or tricuspid valve TV. For example, FIG. 8A is a highly schematic illustration of a cross-section of a mitral valve MV to which two fixation devices 200 have been applied, with a prosthetic heart valve 300 deployed into the mitral valve MV to engage the two fixation devices 200 for anchoring. FIG. 8A also shows an optional occluder 500 deployed between one of the fixation devices and the corresponding native commissure.

In the example of FIG. 8A, a single fixation device 200 has been applied to the pair of mitral valve leaflets AL, PL near the anterolateral commissure ALC, and a single fixation device 200 has been applied to the pair of mitral valve leaflets AL, PL near the posteromedial commissure PMC. Referring briefly to the FIG. 8D, which is a flowchart showing example steps of a prosthetic heart valve implantation method 400E, the fixation devices may be applied in a first step 410E. As with other examples herein, the fixation devices may be applied as part of a single overall procedure that includes the prosthetic heart valve implantation in step 430E, or the fixation devices may have been applied previously as part of a separate procedure days, weeks, months, or years earlier than the other steps of method 400E, described in greater detail below. The fixation devices applied to the pair(s) of leaflets in step 410E may take the form of fixation device 200 described above in connection with FIG. 3, or may take any other suitable form that provides for leaflet engagement and/or approximation of free edges FE of leaflets, for example by pinching, sandwiching, or otherwise clipping the free edges FE of native leaflets together. Further, although method 400E is described in the context of implanting a prosthetic mitral valve, in other examples the method may be similarly performed but with implantation of a prosthetic tricuspid valve. If the valve being replaced is the mitral valve MV, it may be preferable to have at least one fixation device applied near each of the mitral valve commissures, but in other examples at least one fixation device may only be applied near only a single one of the commissures. Similarly, if the valve being replaced is the tricuspid valve TV, it may be preferable to have at least one fixation device applied near two or three of the tricuspid valve commissures, but in other examples at least one fixation device may only be applied near only a single one of the commissures.

Referring again to FIG. 8D, after the fixation devices are applied in step 410E, a prosthetic heart valve is implanted into the native valve in step 430E. Referring again to FIG. 8A, in which two fixation devices 200 have been applied to the leaflets of the mitral valve MV near each of the native commissures, a transcatheter prosthetic heart valve 300 has been implanted into the mitral valve MV (e.g. step 430E of method 400E) so that a main portion of the frame 310 (which houses one, two, or more prosthetic leaflets 370) is positioned in a central portion of the native mitral valve MV between the two fixation devices, and two anchor arms 330 hook from the ventricular side of the native leaflets, through the space between the fixation devices 200 and the corresponding adjacent native commissures, back to the atrial side of the two native leaflets. One example of prosthetic heart valve 300 is shown in isolation (and in cross-section) in FIG. 8B in an expanded or deployed condition. In the illustrated example, the prosthetic heart valve 300 includes a generally cylindrical main frame 310 which may be formed of a shape-memory material, such as a nickel-titanium alloy like nitinol. In some examples, the frame 310 may be cut (e.g. laser cut) from a tube of the shape-memory material and shape-set (e.g. via heat treatment) to the desired shape. In this example, prosthetic heart valve 300 includes three prosthetic leaflets 370 mounted within the frame (although only two are illustrated in FIG. 8B), but it should be understood that the prosthetic heart valve 300 may include any suitable number of prosthetic leaflets 370. The prosthetic heart valve 300 may include one or mor skirts (e.g. inner and/or outer skirts) to assist with sealing and/or mounting components, such as the prosthetic leaflets 370, to the main frame 310. In the illustrated example, a pair of anchor arms 330 extend from the main frame 310 at or near an outflow or ventricular end of the main frame 310. In some examples, the anchor arms 330 are formed integrally with the remainder of the main frame 310. In other examples, the anchor arms 330 may be formed separately, and then attached to (e.g. via sutures) to the main frame 310. In either case, the anchor arms 330 are each preferably formed of a shape-memory material, such as a nickel-titanium alloy such as nitinol. The anchor arms 330 may be shape-set (e.g. via heat treatment) so that the anchor arms 330 extend upwardly (e.g. in the inflow or atrial direction) from their points of attachment to the main frame 310, with the anchor arms 330 terminating in tips 335. In some examples, the tips 335 are formed with atraumatic or blunt features, such as a tissue or fabric padding or a blunted shape so that the anchor arm 330 does not terminate in a sharp end that is likely to pierce into or otherwise damage native tissue. If the prosthetic heart valve 300 includes two anchor arms 330 for use as part of a mitral valve replacement, as in the example of FIG. 8B, they may be positioned at substantially opposite sides of the main frame 300 (e.g. a radial spacing of between about 160 and about 200 degrees, including between about 170 and about 190 degrees, including about 180 degrees). If the prosthetic heart valve 300 includes two or three anchor arms 330 for use as part of a tricuspid valve replacement, two circumferentially adjacent anchor arms 330 may be positioned with a radial spacing of between about 100 and about 140 degrees, including between about 110 and about 130 degrees, including about 120 degrees.

Still referring to FIGS. 8A-8B, when the prosthetic heart valve 300 is in the expanded or deployed condition, a middle portion 333 of each anchor arm 330 (e.g. about halfway the length between the tip 335 of the anchor arm 330 and the base 331 or location where the anchor arm 330 joins the main frame 310) may be positioned farther radially outward from a central longitudinal axis of the main frame 310 than both of the base 331 and the tip 335. With this shape, each anchor arm 330 has a looping shape so that, upon implantation of the prosthetic heart valve 300, the base 331 of the anchor arm 330 may be positioned below (e.g. ventricularly) of a corresponding fixation device 200, the middle portion 333 of the anchor arm 330 may pass through the space between the native leaflets between the fixation device 200 and the adjacent native commissure, and the tip 335 of the anchor arm 335 may be positioned above (e.g. atrially) of the corresponding fixation device 200. In some examples, upon implantation and achieving a configuration similar to that shown in FIG. 8A, the tips 335 of the anchor arms 335 may touch, contact, or otherwise engage with the inflow side (e.g. atrial side) of the corresponding fixation device 200. However, in other examples, the tips 335 may not directly contact the fixation device 200, while still being positioned aligned with the fixation device. With this configuration, the anchor arms 330 may function to prevent migration of the prosthetic heart valve 300 both toward the left atrium LA (during ventricular systole) and toward the left ventricle LV (during ventricular diastole). The same general configuration is applicable to a tricuspid valve replacement, although the number and position of the anchor arms 330 may be tailored to the number and placement of the fixation devices within the native tricuspid valve TV. For example, if three fixation devices are being applied to each leaflet pair of the tricuspid valve TV, the prosthetic heart valve 300 may include three anchor arms 330.

The anchor arms 330 may be deployed in any suitable manner to hook behind and then above the corresponding fixation devices 200, which may be performed prior to or simultaneously with the expansion of the main frame 310 into the orifice of the native mitral valve MV between the fixation devices 200. In some examples, during delivery while the prosthetic heart valve 300 is in the collapsed condition, the anchor arms 330 may be inverted so that the anchor arms extend axially in the outflow direction from the main frame 310, with the tips 335 being positioned at the leading end of the delivery catheter just prior to deployment of the prosthetic heart valve 300. In this example, the tips 335 of the anchor arms 330 may be the first structure of the prosthetic heart valve 300 to exit the delivery catheter, with the anchor arms 330 reverting toward the orientation shown in FIGS. 8A-8B as more and more of the anchor arms 330 clear the delivery catheter. As the anchor arms 330 transition from the inverted condition within the catheter to the reverted (e.g. shape-set) condition outside of the catheter, the anchor arms 330 hook behind and then above the fixation devices 200. Examples of suitable anchor arms, as well as deployment by inversion of the anchor arms, are described in greater detail in U.S. Patent Application Publication Nos. 2016/0278923 and 2020/0179109, the disclosures of which is hereby incorporated by reference herein. In other examples, when the prosthetic heart valve 300 is collapsed within a catheter in a delivery condition, the anchor arms 330 are not inverted, such that the tips 335 extend toward the inflow or atrial end of the prosthetic heart valve 300 both while in a collapsed delivery condition and in an expanded or deployed condition. In these examples, a secondary catheter may cover the anchor arms 330 during delivery which may be withdrawn while the remainder of the prosthetic heart valve 300 remains collapsed within a primary catheter which is at least partially within the left ventricle LV. After the secondary catheter is withdrawn from the anchor arms 330, the anchor arms 330 may expand radially outwards, at which point the prosthetic heart valve may be retracted in the atrial direction to advance the anchor arms 330 through the space between the native leaflets between the fixation device 200 and the corresponding native commissure. After the anchor arms 330 are hooked around the fixation devices, the primary catheter can be withdrawn to allow for the main frame 310 to expand to complete the implantation. Examples of suitable anchor arms, as well as deployment without inversion of the anchor arms, are described in greater detail in U.S. Patent Application Publication No. 2012/0323316, the disclosure of which is hereby incorporated by reference herein.

Referring again to FIG. 8D, in some examples, the method 400E may be complete after steps 410E and 430E are completed. However, in some examples, the space between the fixation device 200 and its corresponding native commissure may become prone to leakage, for example due to the anchor arms 330 extending through this space and/or due to the anchor arms 330 interacting with their corresponding fixation devices 200. Thus, method 400E is illustrated with an optional additional step 440E of implanting an occluder into the space between the native leaflets, radially outward of a fixation device 200 and radially inward of the native commissure adjacent to the fixation device 200. FIG. 8A illustrates on exemplary occluder 500 having been implanted into one of the two spaces. The occluder 500, once implanted, may partially or completely occlude blood from leaking across this small gap space, and the seal may become more complete over time as tissue ingrowth occurs on and/or within the occluder 500. Preferably, if any occluders 500 are implanted in step 440E, they are implanted only after the prosthetic heart valve 300 has been implanted in step 430E. Otherwise, the anchor arm 330 may be blocked from advancing through the gap space between the fixation device 200 and the adjacent native commissure if the occluder 500 is already implanted when the prosthetic heart valve 300 is deployed.

An example of an occluder 500 is shown in FIG. 8C in an expanded condition. Occluder 500 may be formed by cutting (e.g. laser cutting) a tube of shape-memory material, such as a nickel-titanium alloy like nitinol, and shape setting the tube (e.g. via heat treatment) into the desired shape, for example using a mold. In other examples, including that shown in FIG. 8C, the occluder 500 may be formed by braiding or weaving a plurality of strands of shape-memory material, such as a nickel-titanium alloy like nitinol, and shape setting the woven or braided material (e.g. via heat treatment) into the desired shape, for example using a mold. The particular occluder 500 shown in FIG. 8C is shape-set so that, in the absence of applied forces, the occluder 500 includes a first flange 510 and a second flange 520 that are spaced apart from each other and connected by a waist 530. In this particular example, the first flange 510 and the second flange 520 have diameters that are similar or identical to each other, and which are both larger than a diameter of the waist portion 530, with the first flange 510, second flange 520, and waist 530 being continuous and/or integral structures. Also in this particular example, the terminal ends of the strands that form the first flange 510 are gathered and secured (e.g. via a clamping mechanism) by a first clamp or hub 515, and the terminal ends of the strands that form the second flange 520 are gathered and secured (e.g. via a clamping mechanism) by a second clamp or hub 525. One or both of the first hub 515 and second hub 525 may include connection features, such as internal or external threads, that allow for temporary connection to a complementary feature (e.g. matching threads) of a delivery device. It should be understood that an occluder used to occlude the space between a fixation device 200 and the adjacent native commissure may take various other forms, and the illustrated occluder 500 is merely one example. Additional examples of occluders that may be suitable for use as occluder 500, with or without modifications, are described in U.S. Patent Application Publication Nos. 2009/0099647, 2013/0218189, and 2014/0135827, the disclosure of which are hereby incorporated by reference herein.

It should be understood that although a primary goal of method 400E is to achieve anchoring of a prosthetic heart valve based on interaction between anchor arms of the prosthetic heart valve with fixation devices that were previously applied to the leaflets of the native heart valve, other benefits, including any of those described in connection with method 400A, may also be realized by method 400E. Further, if method 400E is being applied to a mitral valve, method 400E may be combined with steps of method 400C or 400D, and if method 400E is being applied to a tricuspid valve, method 400E may be combined with steps of method 400B or 400D. Further, it should be understood that, although the implantation of an occluder 500 to seal potential leaks between a fixation device 200 and its adjacent native commissure is described in step 440E as part of method 400E, if desired, an occluder may be similarly implanted in any of the other methods described herein to address concerns of potential leaks between a fixation device and an adjacent native commissure.

The various methods described above include different devices that are configured for implantation into the heart of a patient via a minimally invasive, transcatheter procedure. For example, the fixation devices (e.g. fixation device 200), the prosthetic heart valves (e.g. prosthetic heart valve 100 or 300), and the occluders (e.g. occluder 500) may all be delivered and deployed using delivery catheters that are sized to pass through the patient's vasculature, allowing for non-surgical implantation of any or all of the devices. As noted herein, in some examples, one or more of the devices described herein may be implanted as part of separate procedures (e.g. procedures that occur days, weeks, months, or years apart from each other), or as part of the same procedure (e.g. a procedure that is performed in the same day and/or in a single location such as a catheter lab setting or surgical site). If multiple devices (e.g. one or more fixation devices and a prosthetic heart valve) are being implanted as part of the same procedure, in some examples each device (or each device type) may be implanted using a catheter or other delivery system specific for the device, but in other examples, at least one delivery system component (such as an introducer) may be used to assist with the delivery of more than one device (or device type) to increase efficiency of the procedure of delivery different devices. For example, U.S. Provisional Patent Application No. 63/724,448, filed Nov. 25, 2024, titled “Multi-Device Steerable Catheter for Multi-Device Implantation,” and which is hereby incorporated by reference herein, describes suitable systems for delivering fixation devices, prosthetic heart valves, and occluders using at least one delivery component in common for the different types of devices.

As noted, in particular in relation to FIGS. 7A-7C, some patients may have a pacemaker, or more than one pacemaker, fitted. The or each pacemaker may be provided with a lead, or more than one lead, which passes from an atrium of the heart to the corresponding ventricle. For example, one or more leads may pass from the right atrium to the right ventricle, passing through the tricuspid valve. By way of further example, one or more leads may pass from the left atrium to the left ventricle, passing through the mitral valve.

The lead or leads may affect the operation of the relevant atrioventricular valve. For example, the lead(s) may prevent leaflets of the valve from closing together, or coapting, fully or properly, with the effect that regurgitation through the incompletely closed valve may occur. In addition, or alternatively, the presence of the lead(s) may interfere with the proper placement and/or configuration of a fixation device, for example in a transcatheter edge-to-edge repair of the mitral valve, or a transcatheter edge-to-edge repair of the tricuspid valve. In addition, or alternatively, the presence of the lead(s) may interfere with the proper placement and/or configuration of a transcatheter replacement valve, for example in a transcatheter tricuspid valve replacement (TTVR) procedure or in a transcatheter mitral valve replacement (TMVR) procedure.

It would therefore be advantageous to mitigate the effect of a lead or leads passing through an atrioventricular valve. For simplicity, reference is now made to examples having one such lead, but it is intended that such references include corresponding examples in which there are two leads, or more than two leads, passing through a given atrioventricular valve.

As described in relation to FIGS. 7A-7C, a fixation device may be applied to two of the native leaflets of an atrioventricular valve at or adjacent the commissure defined between the two leaflets. The location of deployment of the fixation device may be selected to provide a gap, opening, or space, between the fixation device and the commissure. The gap is configured to accommodate a pacemaker lead passing through the atrioventricular valve from the atrium associated with the valve to the ventricle associated with the valve. With the fixation device deployed at or adjacent the commissure, and with the lead passing through the gap, the lead may be secured or held in place. A subsequent atrioventricular valve modification procedure may then be performed. Such procedure may include a transcatheter edge-to-edge valve repair using a second fixation device to engage and approximate two leaflets of the valve. The two leaflets may be the same two leaflets to which the first fixation device is deployed or, if present, another set of two leaflets of the valve. Alternatively, such procedure may include a transcatheter replacement valve.

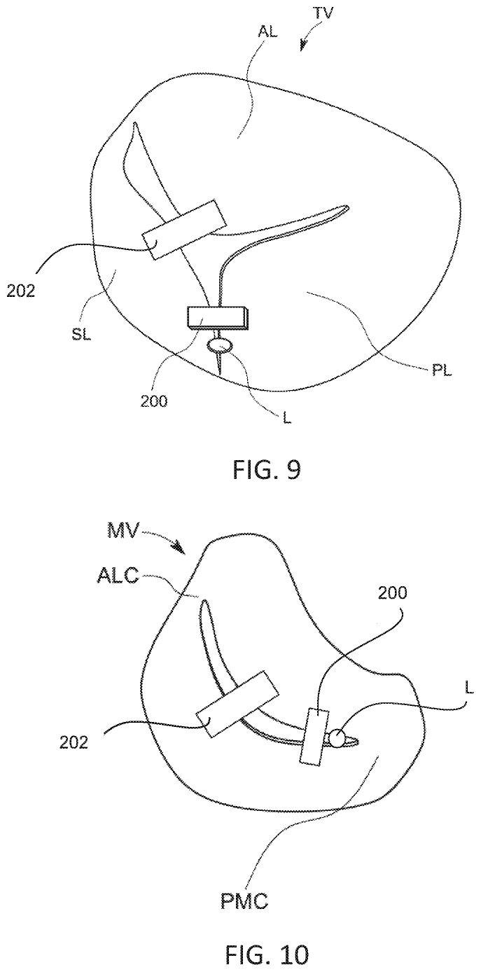

FIG. 9 shows schematically an embodiment in which a first fixation device 200 is used to secure a lead L of a pacemaker and a second fixation device 202 is used to perform a transcatheter edge-to-edge valve repair, in a tricuspid valve TV of a patient. The first fixation device 200 is provided with first fixation elements (not shown) which are configured to engage heart tissue. In the embodiment of FIG. 9, the heart tissue includes a first edge portion of a first native leaflet of the tricuspid valve and a first edge portion of a second native leaflet of the tricuspid valve. The first edge portions are adjacent to a first commissure defined between the two leaflets. In the embodiment of FIG. 9, the leaflets whose first edge portions are engaged are the septal leaflet SL and the posterior leaflet PL. The commissure defined between them is the posteroseptal, or septal-posterior, commissure.

The first edge portions of the leaflets which are engaged by the first fixation device 200 are located adjacent the first commissure so that, when the first fixation device is deployed in position, a gap, or space or opening, is provided between the first commissure and the first fixation device. The gap is configured to have a size suitable for accommodating and/or securing the pacemaker lead L passing from the right atrium to the right ventricle. The lead L may already be generally located at or near the first commissure before deployment of the first fixation device 200. In this case, the first fixation device is used to maintain the position of the lead so that it does not interfere with the operation of the tricuspid valve, or any interference from the lead is low. Alternatively, the lead may not be at or near the first commissure before deployment of the first fixation device 200. In this case, the first fixation device may be configured during deployment to engage and guide the lead L to or towards the first commissure before the first fixation elements engage the first edge portions of the leaflets. The first fixation device may then be deployed to maintain the position of the lead so that it does not interfere with the operation of the tricuspid valve, or any interference from the lead is low.

With the pacemaker lead L secured in the gap adjacent the first commissure, a tricuspid valve repair procedure may be performed. In particular, a second fixation device 202 for reducing tricuspid valve regurgitation may be deployed in the tricuspid valve. The second fixation device 202 is provided with second fixation elements (not shown) which are configured to engage second edge portions of two native leaflets of the tricuspid valve. In the embodiment of FIG. 9, the second fixation device 202 is deployed on the septal leaflet and anterior leaflet pair. While the second fixation device 202 may be deployed at a range of locations between the leaflet pair, the second fixation device 202 in the embodiment shown is located at a generally intermediate position along the edges of the leaflets. The selection of the deployment position of the second fixation device 202 may be guided by where through the leaflet pair regurgitation has been identified to be addressed.

While the embodiment of FIG. 9 illustrates the first fixation device 200 being deployed adjacent the posteroseptal commissure and engaging first edge portions of the septal leaflet SL and the posterior leaflet PL, the first fixation device may alternatively be deployed adjacent other commissures of the tricuspid valve TV. For example, the first fixation device 200 may be deployed to engage first edge portions of the septal leaflet SL and the anterior leaflet AL, adjacent the anteroseptal, or septal-anterior, commissure defined between them. By way of further example, the first fixation device 200 may be deployed to engage first edge portions of the anterior leaflet AL and the posterior leaflet PL, adjacent the anteroposterior, or anterior-posterior, commissure defined between them.

Furthermore, while the embodiment of FIG. 9 illustrates the second fixation device 202 being deployed to engage second edge portions of the septal leaflet SL and the anterior leaflet AL, the second fixation device may alternatively be deployed to engage other leaflet pairs of the tricuspid valve TV. For example, the second fixation device 202 may be deployed to engage second edge portions of the anterior leaflet AL and the posterior leaflet PL. By way of further example, the second fixation device 202 may be deployed to engage second edge portions of the posterior leaflet PL and the septal leaflet SL. In each of these cases, the second fixation device 202 is deployed further away from the commissure adjacent which the first fixation device 200 is deployed. In other words, the first edge portions of the leaflets engaged by the first fixation device 200 are adjacent the first commissure and the second edge portions of the leaflets engaged by the second fixation device 202 are located away from the first commissure.

While the embodiment of FIG. 9 illustrates the first fixation device 200 being deployed adjacent the posteroseptal commissure and engaging first edge portions of the septal leaflet SL and the posterior leaflet PL, the first fixation device may alternatively be deployed adjacent other commissures of the tricuspid valve TV. For example, the first fixation device 200 may be deployed to engage first edge portions of the septal leaflet SL and the anterior leaflet AL, adjacent the anteroseptal, or septal-anterior, commissure defined between them. By way of further example, the first fixation device 200 may be deployed to engage first edge portions of the anterior leaflet AL and the posterior leaflet PL, adjacent the anteroposterior, or anterior-posterior, commissure defined between them.

Furthermore, while the embodiment of FIG. 9 illustrates the second fixation device 202 being deployed to engage second edge portions of the septal leaflet SL and the anterior leaflet AL, the second fixation device may alternatively be deployed to engage other leaflet pairs of the tricuspid valve TV. For example, the second fixation device 202 may be deployed to engage second edge portions of the anterior leaflet AL and the posterior leaflet PL. By way of further example, the second fixation device 202 may be deployed to engage second edge portions of the posterior leaflet PL and the septal leaflet SL. In each of these cases, the second fixation device 202 is deployed further away from the commissure adjacent which the first fixation device 200 is deployed. In other words, the first edge portions of the leaflets engaged by the first fixation device 200 are adjacent the first commissure and the second edge portions of the leaflets engaged by the second fixation device 202 are located away from the first commissure.

FIG. 10 shows schematically an embodiment in which a first fixation device 200 is used to secure a lead L of a pacemaker and a second fixation device 202 is used to perform a transcatheter edge-to-edge valve repair, in a mitral valve MV of a patient. The devices and steps described in relation to FIG. 9 may be applied correspondingly to the embodiment of FIG. 10.

The first fixation device 200 is provided with first fixation elements (not shown) which are configured to engage heart tissue. In the embodiment of FIG. 10, the heart tissue includes a first edge portion of a first native leaflet of the mitral valve and a first edge portion of a second native leaflet of the mitral valve. The first edge portions are adjacent to a first commissure defined between the two leaflets. In the embodiment of FIG. 10, the leaflets whose first edge portions are engaged are the anterior leaflet and the posterior leaflet. The commissure defined between them is the anteroposterior, or anterior-posterior, commissure.

The first edge portions of the leaflets which are engaged by the first fixation device 200 are located adjacent the first commissure so that, when the first fixation device is deployed in position, a gap, or space or opening, is provided between the first commissure and the first fixation device. The gap is configured to have a size suitable for accommodating and/or securing the pacemaker lead L passing from the left atrium to the left ventricle. The lead L may already be generally located at or near the first commissure before deployment of the first fixation device 200. In this case, the first fixation device is used to maintain the position of the lead so that it does not interfere with the operation of the mitral valve, or any interference from the lead is low. Alternatively, the lead may not be at or near the first commissure before deployment of the first fixation device 200. In this case, the first fixation device may be configured during deployment to engage and guide the lead L to or towards the first commissure before the first fixation elements engage the first edge portions of the leaflets. The first fixation device may then be deployed to maintain the position of the lead so that it does not interfere with the operation of the mitral valve, or any interference from the lead is low.

With the pacemaker lead L secured in the gap adjacent the first commissure, a mitral valve repair procedure may be performed. In particular, a second fixation device 202 for reducing mitral valve regurgitation may be deployed in the mitral valve. The second fixation device 202 is provided with second fixation elements (not shown) which are configured to engage second edge portions of the two native leaflets of the mitral valve. The second edge portions are further away from the first commissure than the first edge portions. While the second fixation device 202 may be deployed at a range of locations between the leaflet pair, the second fixation device 202 in the embodiment shown is located at a generally intermediate position along the edges of the leaflets. The selection of the deployment position of the second fixation device 202 may be guided by where through the leaflet pair regurgitation has been identified to be addressed.

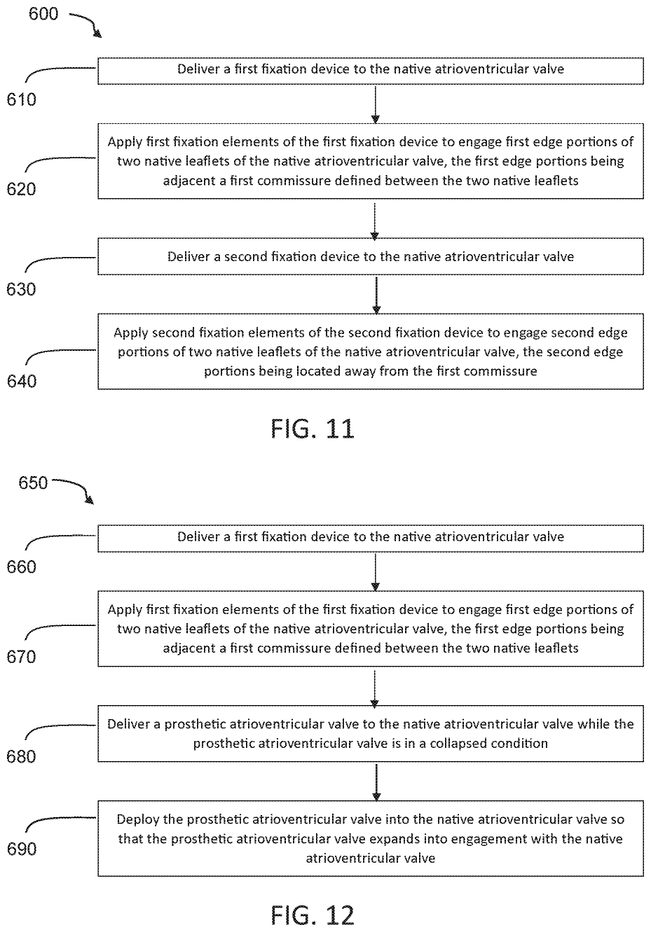

FIG. 11 shows an embodiment of a method 600 of modifying a native atrioventricular valve of a patient which may be implemented to arrive at the configuration of devices illustrated in FIG. 9 or in FIG. 10. Step 610 of method 600 includes delivering a first fixation device to the native atrioventricular valve. The first fixation device is provided with first fixation elements for engaging heart tissue. The atrioventricular valve may be the tricuspid valve or the mitral valve.

Step 620 of method 600 includes applying the first fixation elements of the first fixation device to engage first edge portions of two native leaflets of the native atrioventricular valve. The first edge portions of the leaflets are adjacent a first commissure defined between the two native leaflets. In the case of the tricuspid valve, the leaflets may be any two of the leaflets selected from the anterior leaflet, the posterior leaflet, and the septal leaflet. In the case of the mitral valve, the leaflets are the anterior leaflet and the posterior leaflet.

As part of step 620, the first fixation device may be positioned adjacent the first commissure. The position of the first fixation device is selected to provide a gap, or space or opening, between the first commissure and the first fixation device. The gap is configured to accommodate and/or secure a pacemaker lead or leads passing through the atrioventricular valve. During positioning of the first fixation device, the lead(s) may be engaged and/or repositioned by the first fixation device to move the lead(s) to or towards the first commissure. Alternatively, if the lead(s) is already generally located at or near the first commissure, the first fixation device may engage the lead(s) to maintain the lead(s) at or adjacent the first commissure. In this way, when the first fixation device is deployed and the first fixation elements have engaged the first edge portions of the leaflets, the lead(s) may be maintained and/or secured in position at or adjacent the first commissure, in the gap provided.

Step 630 of method 600 includes delivering a second fixation device to the native atrioventricular valve. The second fixation device is provided with second fixation elements for engaging and approximating second edge portions of two leaflets in the valve. The second fixation device is configured to repair the valve by reducing regurgitation therethrough.

Step 640 of method 600 includes applying the second fixation elements to engage the second edge portions of the two native leaflets of the native atrioventricular valve. The second edge portions are located away from the first commissure. In other words, the second edge portions are located further away from the first commissure than the first edge portions. In the case of the mitral valve, the first and second edge portions are on the same two leaflets of the valve; namely, the anterior leaflet and the posterior leaflet. In the case of the tricuspid valve, the first and second edge portions may be on the same two leaflets of the valve, but are not necessarily so. The first edge portions may be located on any selected two of the anterior leaflet, the posterior leaflet, and the septal leaflet, adjacent or close to the respective first commissure defined between the selected leaflets. The second edge portions may be located on any selected two of the anterior leaflet, the posterior leaflet, and the septal leaflet. The location of the second edge portions is further away from the first commissure than the location of the first edge portions.

In this way, a second fixation device 202 may be deployed to repair a native atrioventricular valve, in particular by reducing regurgitation through the valve, without the presence of a pacemaker lead(s) interfering with the positioning, deployment, and/or functionality of the fixation device. In addition, or alternatively, a fixation device 202 for addressing atrioventricular valve regurgitation may be deployed without disturbing, catching on, or interfering with a pacemaker lead(s) passing through the valve.

FIG. 12 shows an embodiment of a method 650 of modifying a native atrioventricular valve of a patient which may be implemented to arrive at the configuration of devices, and use of method 400D, illustrated in FIGS. 7A-7C. While FIGS. 7A-7B illustrate an embodiment applied to the tricuspid valve, the method may correspondingly be applied to the mitral valve of a patient.

Steps 660 and 670 of method 650 correspond to steps 610 and 620 of method 600, so reference is made to the description of method 600 and steps 610, 620, above.

Step 680 of method 650 includes delivering a prosthetic atrioventricular valve to the native atrioventricular valve while the prosthetic atrioventricular valve is in a collapsed condition.

Step 690 of method 650 includes deploying the prosthetic atrioventricular valve into the native atrioventricular valve so that the prosthetic atrioventricular valve expands into engagement with the native atrioventricular valve.

In this way, a prosthetic atrioventricular valve may be deployed to replace a native atrioventricular valve without the presence of a pacemaker lead(s) interfering with the positioning, deployment, and/or functionality of the prosthetic atrioventricular valve. In addition, or alternatively, a prosthetic atrioventricular valve may be deployed without disturbing, catching on, or interfering with a pacemaker lead(s) passing through the valve.

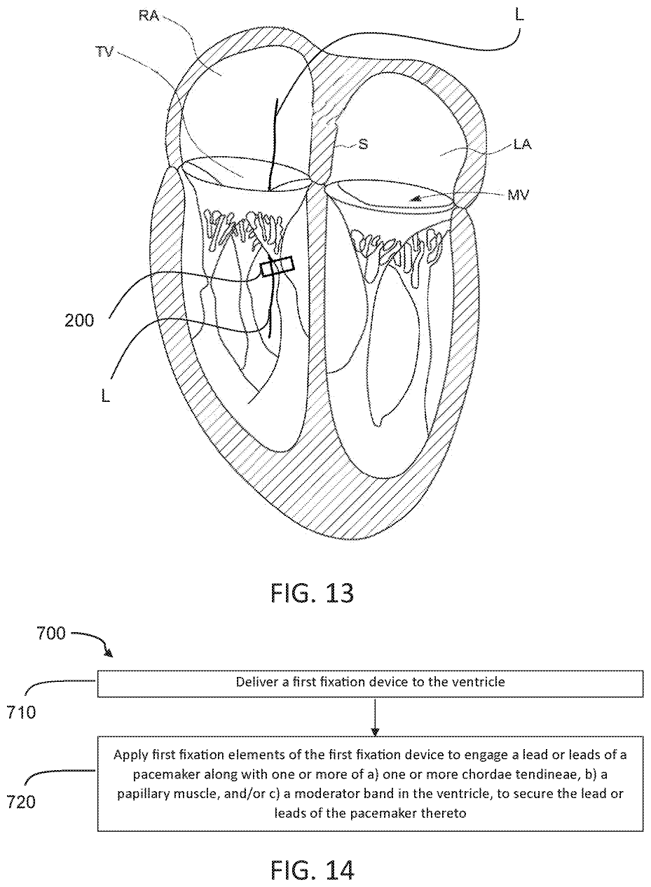

FIG. 13 shows schematically an embodiment in which a first fixation device 200 is used to secure a lead L of a pacemaker to heart tissue in the right ventricle of a patient's heart. The first fixation device 200 is provided with first fixation elements (not shown) which are configured to engage the heart tissue. In the embodiment of FIG. 13, the heart tissue includes one or more chordae tendineae extending from one of the leaflets of the atrioventricular valve to one of the papillary muscles in the ventricle. Depending on the initial location of the pacemaker lead(s) and/or the desired secured location for the lead(s), the fixation device 200 may be configured to engage any group or sub-group of one or more chordae tendineae. In other embodiments, the first fixation device 200 may be configured alternatively or additionally to engage a papillary muscle in the ventricle and/or a moderator band in the ventricle, if present. Generally, a moderator band may be found in the right ventricle of the heart. However, in some patients, there may be left ventricle bands or fibromuscular structures present, and one or more of these structures may be referred to as a moderator band.

In addition to engaging the ventricular heart tissue, the first fixation device 200 is configured to engage the pacemaker lead(s) L passing through the tricuspid valve from the right atrium to the right ventricle. In this way, the first fixation device 200 may be deployed to engage both the lead(s) and the heart tissue to secure the lead(s) to the tissue.

The selection of the heart tissue to engage—namely, one or more of a) one or more chordae tendineae, b) a papillary muscle, and/or c) a moderator band in the ventricle may depend on the initial location of the lead(s). The lead(s) L may already be generally located at or near a commissure of one of the heart valve leaflet pairs before deployment of the first fixation device 200. In this case, the first fixation device is used to maintain the position of the lead(s) so that it does not interfere with the operation of the tricuspid valve, or any interference from the lead is low. Alternatively, the lead(s) may not be at or near a commissure before deployment of the first fixation device 200. In this case, the first fixation device may be configured during deployment to engage and guide the lead(s) L to or towards a commissure before the first fixation elements engage the heart tissue. The first fixation device may then be deployed to maintain the position of the lead(s) so that it does not interfere with the operation of the tricuspid valve, or any interference from the lead is low.

With the pacemaker lead(s) L secured with the heart tissue by the first fixation device 200, a tricuspid valve repair or replacement procedure may be performed. For example, a second fixation device 202 for reducing tricuspid valve regurgitation may be deployed in the tricuspid valve. By way of further example, a prosthetic atrioventricular valve may be deployed in the tricuspid valve. Correspondingly, when applied to the mitral valve, with the pacemaker lead(s) L secured with the heart tissue by the first fixation device 200, a mitral valve repair or replacement procedure may be performed. For example, a second fixation device 202 for reducing mitral valve regurgitation may be deployed in the mitral valve. By way of further example, a prosthetic atrioventricular valve may be deployed in the mitral valve.

FIG. 14 shows an embodiment of a method 700 of modifying a native atrioventricular valve of a patient which may be implemented to arrive at the configuration of devices illustrated in FIG. 13 or described above. Step 710 of method 700 includes delivering a first fixation device to the ventricle of a patient's heart. The first fixation device is provided with first fixation elements for engaging heart tissue. The atrioventricular valve may be the tricuspid valve or the mitral valve and the ventricle may be the right ventricle or the left ventricle, respectively.

Step 720 of method 700 includes applying the first fixation elements of the first fixation device 200 to engage a lead or leads of a pacemaker, along with one or more of a) one or more chordae tendineae, b) a papillary muscle, and/or c) a moderator band in the ventricle, to secure the lead or leads of the pacemaker thereto.

As part of step 720, during positioning of the first fixation device, the lead(s) may be engaged and/or repositioned by the first fixation device to move the lead(s) to or towards a commissure defined between two native leaflets of the native atrioventricular valve. Alternatively, if the lead(s) is already generally located at or near the commissure, the first fixation device may engage the lead(s) to maintain the lead(s) at or adjacent the commissure. In this way, when the first fixation device is deployed and the first fixation elements have engaged the lead(s) and the ventricular heart tissue, the lead(s) may be maintained and/or secured in position at or adjacent the commissure.

With the pacemaker lead(s) L secured with the heart tissue by the first fixation device 200, an atrioventricular valve repair or replacement procedure may be performed. For example, a second fixation device 202 for reducing atrioventricular valve regurgitation may be deployed in the valve. By way of further example, a prosthetic atrioventricular valve may be deployed in the atrioventricular valve.

In this way, a second fixation device 202 may be deployed to repair a native atrioventricular valve, in particular by reducing regurgitation through the valve, without the presence of a pacemaker lead(s) interfering with the positioning, deployment, and/or functionality of the fixation device. In addition, or alternatively, a fixation device 202 for addressing atrioventricular valve regurgitation may be deployed without disturbing, catching on, or interfering with a pacemaker lead(s) passing through the valve. Additionally or alternatively still, a prosthetic atrioventricular valve may be deployed to replace a native atrioventricular valve without the presence of a pacemaker lead(s) interfering with the positioning, deployment, and/or functionality of the prosthetic atrioventricular valve. In addition, or alternatively, a prosthetic atrioventricular valve may be deployed without disturbing, catching on, or interfering with a pacemaker lead(s) passing through the valve.

In any one or more of the embodiments described above and/or in connection with FIGS. 7A-7C and 9-14, the first fixation device 200 may be as shown and/or described in connection with FIG. 3. Additional details of examples of fixation device 200 are provided in U.S. Patent Application Publication No. 2024/0382308, the disclosure of which is hereby incorporated by reference herein. For example, the first fixation device 200 for securing a pacemaker lead(s) L may take the same form as a second fixation device 202 for reducing atrioventricular valve regurgitation.

Alternatively, the first fixation device 200 may be smaller than the second fixation device 202. The first fixation device 200 may be smaller than a known, commercially available fixation device. Such a known fixation device may be the MitraClip® G4 NT fixation device, available from Abbott Laboratories. The second fixation device 202 may be a known, commercially available fixation device. For example, the second fixation device 202 may be the MitraClip® G4 NT fixation device.

The first fixation device 200 may have generally the same or a similar form or structure as the second fixation device 202, but may be configured with smaller overall dimensions. This may provide an advantage in the first fixation device 200 taking up relatively less space than the second fixation device 202. At the same time, since the heart tissue the first fixation device 200 is configured to engage may have a smaller extent than the free edges of valve leaflets the second fixation device 202 is configured to engage, the first fixation device 200 being of smaller dimensions does not affect its function. In other words, the amount of heart tissue to be engaged at or near the edges of atrioventricular leaflets adjacent the commissure defined between those leaflets, or the amount of heart tissue for engagement with a pacemaker lead(s) when the tissue is one or more chordae tendineae, a papillary muscle, and/or a moderator band, may be relatively less.

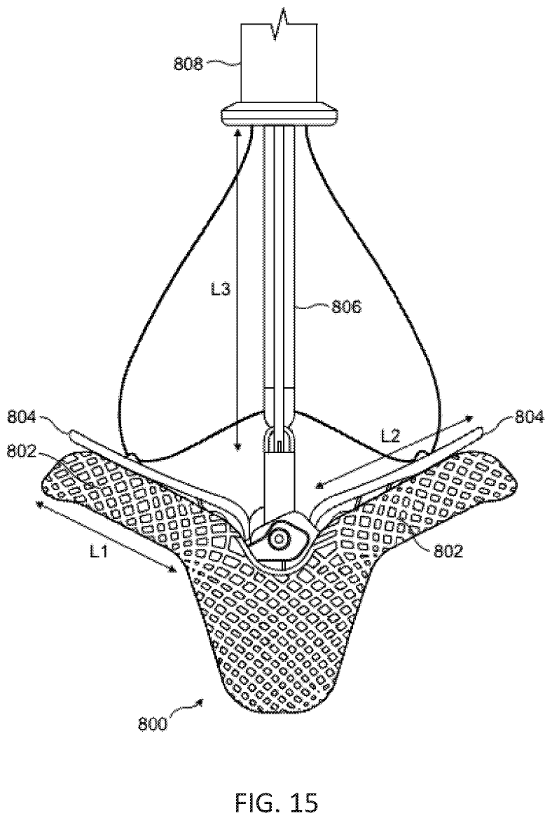

The first fixation device 200 may be configured to be smaller than the second fixation device 202 in one or more of a number of ways. The length of the fixation elements of the first fixation device 200 may be shorter than the length of the fixation elements of the second fixation device 202. Additionally or alternatively, the fixation devices may be provided with gripping elements to cooperate with the fixation elements to engage heart tissue, and the length of the gripping elements of the first fixation device 200 may be shorter than the length of the gripping elements of the second fixation device 202. Additionally or alternatively still, the fixation devices may be releasably coupled to a coupling member of a delivery shaft for delivering the fixation devices to the heart, and the length of the coupling member for the first fixation device 200 may be shorter than the length of the coupling member for the second fixation device 202. These dimensional configurations are discussed in more detail in relation to FIGS. 15-23.

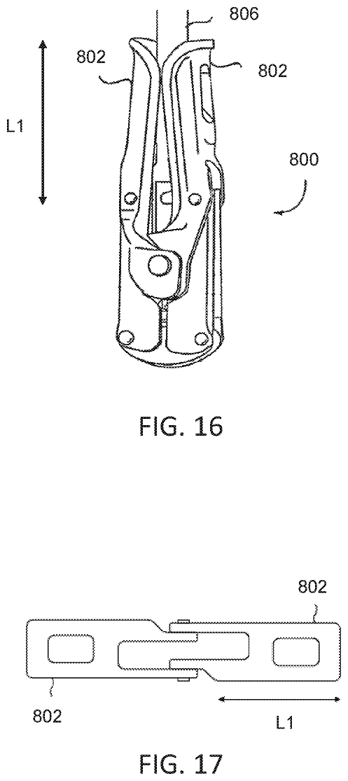

FIG. 15 shows a side view of an exemplary fixation device 800 for engaging and grasping heart tissue. The fixation device 800 has fixation elements 802 and gripping elements 804. A respective fixation element 802 and gripping element 804 are moveable relative to one another to capture and grasp heart tissue therebetween. In the example shown in FIG. 15, part of the fixation device 800, including the fixation elements 802, is covered with a biocompatible covering. The covering is, however, optional and may be omitted. FIG. 16 illustrates such a fixation device 800 provided without a covering, or provided before a covering has been applied.

Each fixation element 802 has a length L1 over which the fixation element is effective to cooperate with a gripping element 804 to engage tissue. In other words, the length L1 represents the tissue engagement length for a fixation element 802. FIG. 17 illustrates a plan view of the fixation elements 802 of the fixation device 800, in which the fixation elements are shown in isolation from the remainder of the fixation device. The portion of the fixation element 802 for tissue engagement is indicated with length L1.

A first implementation of fixation device 800 may be provided as the first fixation device 200. A second implementation of fixation device 800 may be provided as the second fixation device 202. Advantageously, the length L1 in the first fixation device 200 may be configured to be shorter than the length L1 in the second fixation device 202. This can offer a smaller first fixation device 202 which occupies less space in the heart. This offers the benefit of repositioning and/or securing a pacemaker lead(s) out of the way, to allow the second fixation device to be delivered and deployed more straightforwardly, while also keeping the impact of the presence of the first fixation device on the delivery and/or deployment of the second fixation device low.

In an advantageous configuration, the first fixation element length may be 0.05 inches (1.3 mm) shorter than the second fixation element length. In an optional configuration, the first fixation element length may be 0.265 inches (6.73 mm) and the second fixation element length may be 0.315 inches (8.00 mm).

Referring back to FIG. 15, each gripping element 804 has a length L2 over which the gripping element is effective to cooperate with a fixation element 802 to engage tissue. In other words, the length L2 represents the tissue engagement length for a gripping element 804. A first implementation of fixation device 800 may be provided as the first fixation device 200. A second implementation of fixation device 800 may be provided as the second fixation device 202. Advantageously, the length L2 in the first fixation device 200 may be configured to be shorter than the length L2 in the second fixation device 202. This can offer a smaller first fixation device 202 which occupies less space in the heart. This offers the benefit of repositioning and/or securing a pacemaker lead(s) out of the way, to allow the second fixation device to be delivered and deployed more straightforwardly, while also keeping the impact of the presence of the first fixation device on the delivery and/or deployment of the second fixation device low.

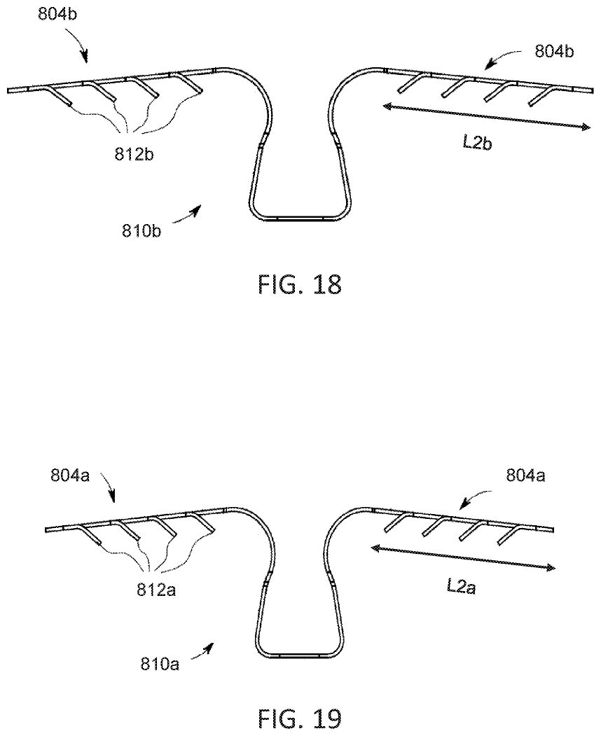

FIG. 18 illustrates a side view of gripping elements 804b of the fixation device 800 when implemented as the second fixation device 202. The gripping elements 804b are provided as part of a gripper structure 810b, which is shown in FIG. 18 in isolation from the remainder of the fixation device. In this configuration, the gripper structure 810b is provided as an integral structure including the two gripper elements 804b and a coupling portion therebetween. The coupling structure 810b may be formed from a shape memory material, such as Nitinol or the like. In other configurations, the gripper elements 810b may be provided separately and coupled together. Each of the gripping elements 804b is provided with a plurality of friction elements 812b. The frictional elements 812b may have the form of barbs, prongs, tines, or the like. The frictional elements 812b may be provided separately from the gripping elements 804b and coupled thereto. Advantageously, the frictional elements 812b are integrally formed with the gripping elements 804b, such as by being laser cut or mechanically cut from the material of the gripper structure 810b and bent or shaped into position. The frictional elements 812b are configured to engage and at least temporarily secure heart tissue. In the example of FIG. 18, each gripping element 804b includes four frictional elements 812b on each side of the gripping element. Because of the side view illustrated in FIG. 18, only the near-side frictional elements are apparent, while the far-side frictional elements are obscured by the near-side frictional elements. However, the number of frictional elements may be varied to be one, two, three, five, six, or more than six frictional elements on each side of a gripping element. The configuration of the frictional elements may also be varied so that frictional elements are provided on only one side of each gripping element, or are provided at an intermediate location across the width of the gripping element. In FIG. 18, the portion of a gripping element 804b for tissue engagement is indicated with length L2b.

FIG. 19 illustrates a side view of gripping elements 804a of the fixation device 800 when implemented as the first fixation device 200. The description of the gripping elements and gripper structure in relation to FIG. 18 applies correspondingly to FIG. 19, but with the reference numerals ending with a letter ‘a’ in FIG. 19, rather than the letter ‘b’ as in FIG. 18. It can be seen that the gripping elements 804a are smaller than the gripping elements 804b. In other words, the length L2a is shorter than the length L2b. The gripping elements 804a are shown with the same number of frictional elements 812a as the number of frictional elements 812b on the gripping elements 804b, but the frictional elements 812a are spaced more closely together than the frictional elements 812b.

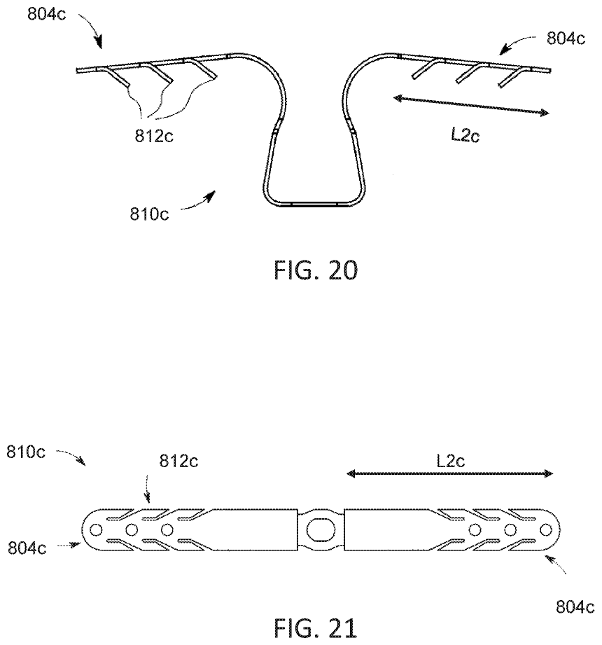

FIGS. 20-21 show an alternative configuration of gripping elements for the first fixation device 200 in which the length of the gripping elements is shorter than the length of the gripping elements of the second fixation device. FIG. 20 shows a side view and FIG. 21 shows a plan view of the alternative configuration. In this configuration, the gripping elements 804c of the first fixation device 200 are provided with fewer frictional elements 812c than the gripping elements 804b of the second fixation device 202. As shown, each gripping element 804b may be provided with four frictional elements 812b on each side of the gripping element. However, each gripping element 804c may be provided with three frictional elements 812c on each side of the gripping element. In this way, the length L2c of the gripping element 804c may be shorter than the length L2b of the gripping element 804b. The total number and/or configuration of frictional elements 812b on a gripping element 804b may be varied as explained above. Whatever the number and/or configuration of frictional elements 812b on the gripping elements 804b of the second fixation device 202, the number of frictional elements 812c on the gripping elements 804c of the first fixation device 200 may be lower.

In an advantageous configuration, the gripping element length L2b is 0.05 inches (1.3 mm) shorter than the gripping element length L2 a and/or L2 c. In an optional configuration, the gripping element length L2 b is 0.256 inches (6.50 mm) and the gripping element length L2 a is 0.306 inches (7.77 mm). In another optional configuration, the gripping element length L2 b is 0.256 inches (6.50 mm) and the gripping element length L2 c is 0.306 inches (7.77 mm).

The gripping element configurations shown in FIGS. 19-21 offer a smaller first fixation device 202 which occupies less space in the heart. This offers the benefit of repositioning and/or securing a pacemaker lead(s) out of the way, to allow the second fixation device to be delivered and deployed more straightforwardly, while also keeping the impact of the presence of the first fixation device on the delivery and/or deployment of the second fixation device low.

The reduction in size of a gripping element and/or the reduction in number of frictional elements on the gripping element may be combined with the reduction in size of a fixation element in the first fixation device. The above advantages also apply to such combination.

Referring back to FIG. 15, the fixation device 800 is shown releasably coupled to a coupling member 806 of a delivery catheter 808 for delivering the fixation device to the heart. The coupling member 806 has a length L3 from the distal end of the delivery catheter 808 to the distal end of the coupling member, where the coupling member releasably couples to the fixation device. A first implementation of fixation device 800 may be provided as the first fixation device 200, with a first delivery catheter being provided for delivering the first fixation device. A second implementation of fixation device 800 may be provided as the second fixation device 202, with a second delivery catheter being provided for delivering the second fixation device. Advantageously, the length L3 of the coupling member 806 for coupling the first delivery catheter to the first fixation device 200 may be configured to be shorter than the length L3 of the coupling member 806 for coupling the second delivery catheter to the second fixation device 202. This can advantageously offer a smaller delivery profile for the first fixation device 200.

In an advantageous configuration, the first coupling member length L3 for the coupling member for the first fixation device is 0.05 inches (1.3 mm) shorter than the second coupling member length L3 for the coupling member for the second fixation device. In an optional configuration, the first coupling member length is 0.356 inches (9.04 mm) and the second coupling member length is 0.406 inches (10.3 mm).

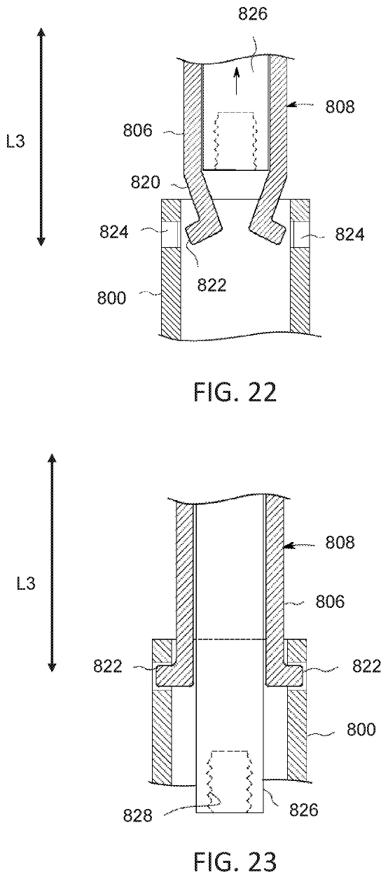

Any of the coupling members 806 described herein may be configured to releasably couple to a corresponding fixation device 800 by means of a coupling mechanism. The releasable coupling mechanism may be provided in a number of ways, including by means of a threaded coupling, a pin and slot coupling, a snap fit coupling, a friction fit coupling, a magnetic coupling, and the like. As shown in FIGS. 22-23, the releasable coupling mechanism 820 may include one of the coupling member 806 or the fixation device 800 being provided with moveable members 820 with outwardly protruding portions 822, such as L-shaped legs, which releasably engage with corresponding recesses or openings 824 in the other of the coupling device or the fixation device. The moveable members 820 may be biased inwards, to disengage from the corresponding recesses or openings 824, so that the coupling member 806 and the fixation device 800 may be released from each other when desired. A locking member 826 may be provided to force the moveable members 820 outwards and into engagement with the corresponding recesses or openings 824, to maintain the fixation device 800 coupled to the coupling member 806 until release is desired. The locking member 826 may be provided in the form of an actuator rod passing through the coupling member 806, between the moveable members 820. When the locking member 826 is located between the moveable members 820, the members are forced outwards and their protruding portions 822 may releasably couple to the corresponding recesses or openings 824. When the locking member 826 is removed, such as being withdrawn axially, the members 820 are configured to move inwards and decouple from the corresponding recesses or openings 824, so that the coupling member 806 and the fixation device 800 may be separated.

Advantageously, the actuator rod 826 may provide another function in addition to locking and unlocking the coupling member 806 to the fixation device 800. The distal end of the actuator rod 826 may be provided with a coupling mechanism 828, such as a threaded engagement mechanism, to releasably couple to the fixation device 800. Such coupling may allow the actuator rod to control the position and/or deployment of the fixation device.

The reduction in size of a coupling member for a delivery catheter to releasably couple to the first fixation device may be combined with a) the reduction in size of a gripping element and/or the reduction in number of frictional elements on the gripping element in the first fixation device, and/or b) the reduction in size of a fixation element in the first fixation device. The above advantages also apply to such combination.

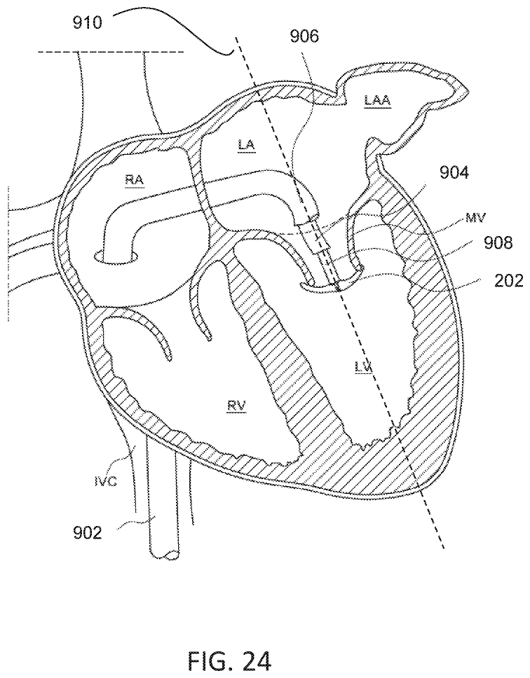

Delivery of the second fixation device 202, to engage and approximate valve leaflets to reduce valve regurgitation, may be achieved in a number of ways. One way is described in relation to FIG. 24, illustrating delivery to the mitral valve. Corresponding procedures may be implemented for delivery to the tricuspid valve, as will be appreciated. FIG. 24 shows a schematic cross section of a heart, including the right atrium (“RA”), the left atrium (“LA”), the right ventricle (“RV”), the left ventricle (“LV”), the mitral valve (“MV”) between the left atrium and the left ventricle, and the inferior vena cava (“IVC”), among other parts. Access to the heart is typically achieved via a remote vascular access site, such as the femoral vein. A typical procedure involves the following steps. A guidewire (not shown) is passed through the vasculature from the remote access site, through the inferior vena cava, and into the right atrium. The guidewire is directed to the interatrial septum, which it penetrates by means of a sharp or cutting tip, to enter the left atrium. Once there, an outer guide catheter (not shown) is passed over the guidewire to the left atrium. A dilator (not shown) may be used with the outer guide catheter to open up, and/or maintain open, the pathway for the outer guide catheter, including for passing through the interatrial septum and into the left atrium. When the outer guide catheter is in position, the optional dilator, if used, may then be withdrawn.

The outer guide catheter has a lumen therethrough, through which a fixation device delivery system may be delivered to the left atrium. The fixation device delivery system includes an inner guide catheter 902 and a delivery catheter 904. The inner guide catheter 902 is steerable to position a distal end 906 of the inner guide catheter 902 in a desired orientation within the left atrium. The inner guide catheter 902 has a lumen therethrough, through which the delivery catheter 904 may be delivered to the left atrium. The delivery catheter 904 is releasably coupled to a fixation device 202 which is to be implanted at the atrioventricular valve, in this case the mitral valve. The releasable coupling is provided by a coupling member 908. The coupling member 908 is deployable and retractable with the delivery catheter 904. The coupling member 908 may be released from the fixation device 202 when the fixation device is deployed in position at the mitral valve.

The outer guide catheter extends through the interatrial septum and terminates within the left atrium. In this way, the outer guide catheter allows for the delivery of components through its lumen into the left atrium. The inner guide catheter 902 is delivered through the lumen of the outer guide catheter, being guided therethrough to the left atrium, and out of the distal end of the outer guide catheter. At this point, the distal end 906 of the inner guide catheter 902 is not directed towards the mitral valve. The mitral valve is provided between the left atrium and the left ventricle and allows blood to flow from the left atrium to the left ventricle. An inflow axis 910 may be defined through the mitral valve, running from the left atrium to the left ventricle. The entry orientation at which the inner guide catheter 902 enters the left atrium, exiting the outer guide catheter, is not in line with, or parallel with, the inflow axis 910. The entry orientation may be as much as substantially perpendicular, or at 90 °, to the inflow axis 910. The inner guide catheter 902 is provided with a steerable portion which is configured to deflect the inner guide catheter in the left atrium from the entry orientation, not parallel to the inflow axis 910, to a delivery orientation, substantially parallel to the inflow axis. The steerable portion is generally provided at or adjacent the distal end of the inner guide catheter 902. The steerable portion is configured to deflect from a straight, or substantially straight, configuration to a bent configuration comprising a bend of up to 90 °. In this way, the inner guide catheter 902 may be re-oriented to be directed downwards and towards the mitral valve, generally or substantially parallel to the inflow axis 910.

The steerable portion may be controlled by a steering control interface provided at or near the proximal end of the inner guide catheter. The steering control interface may include one or more than one knob, lever, or other rotatable and/or translatable actuator to move the steerable portion. The steerable portion may thereby be moved in a first direction and a second, opposite direction along a first axis. The steerable portion may additionally be moved in a third direction and a fourth, opposite direction along a second axis, the second axis being perpendicular to the first axis. These directions may be referred to as anterior and posterior on a first axis and medial and lateral on a second axis. The directions may alternatively be referred to as forwards and backwards on one axis and left and right on the other axis. The steering control interface is coupled to the steerable portion by a wire assembly which is configured to pull and/or push the steerable portion to deflect the steerable portion as desired. Typically, a steerable ring is provided in the inner guide catheter's wall, with the wire assembly coupling to the steerable ring. One wire may be provided between the steering control interface and the steerable ring for each direction of pull or push desired. Advantageously, four wires are provided, one each at 0 °, 90 °, 180 °, and 270 ° around the ring, so that the ring may be pulled (or pushed, but pulling under tension is generally preferred) in any one or more directions.

With the inner guide catheter 902 oriented at its distal end 906 substantially parallel to the inflow axis 910, the delivery catheter 904 may be deployed beyond the distal end 906 of the inner guide catheter 902 to position the fixation device 202 at the mitral valve as desired. The delivery catheter 904 and fixation device 202 may be delivered through the valve to the left ventricle to position the fixation device 202 below the valve leaflets in order to capture the leaflets. The leaflets may then be engaged by the fixation device 202 and approximated, to grasp the leaflets together at the fixation device. With satisfactory reduction in valve regurgitation being observed through imaging, the fixation device 202 may be released from the coupling member 908 of the delivery catheter 904, to be left in place in the heart. The delivery catheter 904 may then be retracted into the inner guide catheter's lumen. The delivery catheter 904 and the inner guide catheter 902 may then be withdrawn from the patient's body, either one at a time or simultaneously.

Delivery of the first fixation device 200, to trap and/or secure a pacemaker lead or leads, may be performed before delivery of the second fixation device 202. Delivery of the first fixation device 200 may be achieved in a number of ways. One way may use a similar delivery system to that for the second fixation device, but with the system being adapted for the delivery of the first fixation device. As discussed, the first fixation device 200 may be delivered to engage particular heart tissue, including one or more of a) edge portions of two native leaflets of the atrioventricular valve, where the edge portions are adjacent a valve commissure defined between the two native leaflets, b) one or more chordae tendineae, c) a papillary muscle, and/or d) a moderator band. An inner guide catheter and/or a delivery catheter for a first fixation device 200 may be adapted for such delivery.

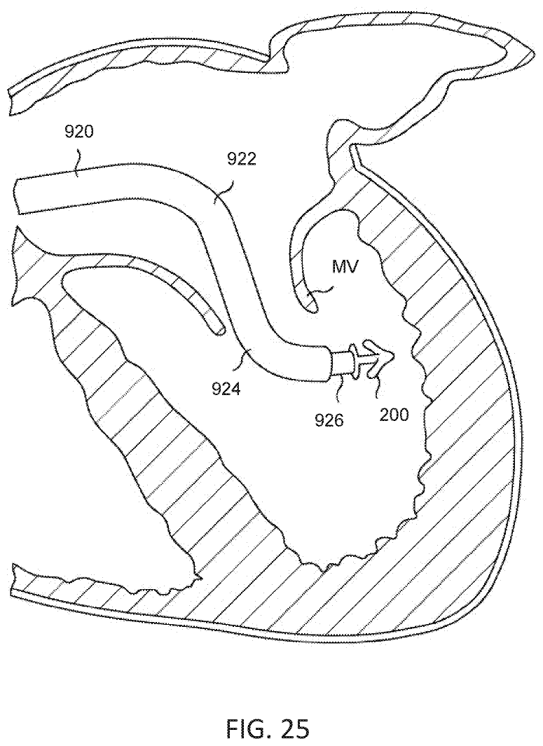

FIG. 25 shows schematically an exemplary implementation, in which a fixation device delivery system is configured to be delivered to the ventricle for delivery of a first fixation device 200 thereto. FIG. 25 shows an example for delivery to the left ventricle. However, a corresponding delivery system may be implemented for delivery to the right ventricle, as will be appreciated. The fixation device delivery system includes an inner guide catheter 920 and a delivery catheter 926. The inner guide catheter 920 is steerable to position a distal end of the inner guide catheter 920 in a desired orientation within the left ventricle. The inner guide catheter 920 has a lumen therethrough, through which the delivery catheter 926 may be delivered to the left ventricle. The delivery catheter 926 is releasably coupled to a first fixation device 200 which is to be used to engage and/or secure a pacemaker lead or leads into or adjacent a valve commissure, as described above. The releasable coupling is provided by a coupling member. The coupling member is deployable and retractable with the delivery catheter 926. The coupling member may be released from the fixation device 200 when the fixation device is deployed in a desired position, to maintain the lead(s) out of the way, at or adjacent an outer edge or annulus of the valve, such as in one of the valve's commissures.

The inner guide catheter 920 may be delivered to the left atrium through an outer guide catheter (not shown), in a similar manner as described in connection with FIG. 24 and delivery of the inner guide catheter 902 for the second fixation device 202. The inner guide catheter 920 is delivered through the lumen of the outer guide catheter, being guided therethrough to the left atrium, and out of the distal end of the outer guide catheter. At this point, the distal end of the inner guide catheter 920 is not directed towards the mitral valve. In other words, the entry orientation at which the inner guide catheter 920 enters the left atrium, exiting the outer guide catheter, is not in line with, or parallel with, the inflow axis of the mitral valve. The entry orientation may be as much as substantially perpendicular, or at 90 °, to the inflow axis. The inner guide catheter 920 is provided with a first steerable portion 922 which is configured to deflect the inner guide catheter in the left atrium from the entry orientation, not parallel to the inflow axis, to a second, inflow orientation, substantially parallel to the inflow axis. The first steerable portion 922 is configured to deflect from a straight, or substantially straight, configuration to a bent configuration comprising a bend of up to 90 °. In this way, the inner guide catheter 920 may be re-oriented to be directed downwards and towards the mitral valve, parallel, or substantially parallel, to the inflow axis.

The first steerable portion 922 may be controlled by a first steering control interface provided at or near the proximal end of the inner guide catheter 920. The first steering control interface may include one or more than one knob, lever, or other rotatable and/or translatable actuator to move the first steerable portion. The first steerable portion may thereby be moved in a first direction and a second, opposite direction along a first axis. The first steerable portion may additionally be moved in a third direction and a fourth, opposite direction along a second axis, the second axis being perpendicular to the first axis. These directions may be referred to as anterior and posterior on a first axis and medial and lateral on a second axis. The directions may alternatively be referred to as forwards and backwards on one axis and left and right on the other axis. The first steering control interface is coupled to the first steerable portion 922 by a wire assembly which is configured to pull and/or push the first steerable portion 922 to deflect the first steerable portion as desired. Typically, a steerable ring is provided in the inner guide catheter's wall, with the wire assembly coupling to the steerable ring. One wire may be provided between the first steering control interface and the steerable ring for each direction of pull or push desired. Advantageously, four wires are provided, one each at 0 °, 90 °, 180 °, and 270 ° around the ring, so that the ring may be pulled (or pushed, but pulling under tension is generally preferred) in any one or more directions.

With the first steerable portion 922 of the inner guide catheter 920 oriented substantially parallel to the inflow axis, the distal end of the inner guide catheter 920 is configured to pass through the mitral valve and into the left ventricle. The inner guide catheter 920 is provided with a second steerable portion 924, distal to the first steerable portion 922. The second steerable portion 924 is configured to deflect the inner guide catheter in the ventricle from the inflow orientation, substantially parallel to the inflow axis, to a third, delivery orientation, not parallel to the inflow axis. The second steerable portion 924 is configured to deflect from a straight, or substantially straight, configuration to a bent configuration comprising a bend of up to 90 °. The third, delivery orientation of the inner guide catheter 920 may be as much as substantially perpendicular, or at 90 °, to the inflow axis. In this way, the inner guide catheter 920 may be re-oriented to be directed towards the heart tissue which the first fixation device 200 is configured to engage. The heart tissue may include one or more of a) edge portions of two native leaflets of the atrioventricular valve, where the edge portions are adjacent a valve commissure defined between the two native leaflets, b) one or more chordae tendineae, c) a papillary muscle, and/or d) a moderator band.

The second steerable portion 924 may be controlled by a second, respective steering control interface provided at or near the proximal end of the inner guide catheter 920. The second steering control interface may include one or more respective knobs, levers, or other rotatable and/or translatable actuators to move the second steerable portion, along with a respective wire assembly and a respective steerable ring, or the like, in a corresponding manner to that described in connection with the first steering control interface.

In view of the space limitations in the ventricle and/or to allow the inner guide catheter 920 to be oriented as desired, the second steerable portion 924 is preferably configured to make a sharp or tight turn. Optionally, the second steerable portion 924 may be configured with a radius of curvature of 0.5 cm to 1 cm.

Additionally or alternatively, the second steerable portion 924 may be disposed adjacent to the distal end of the inner guide catheter 920. Optionally, the second steerable portion 924 may be configured 1 cm to 2 cm from the distal end of the inner guide catheter.

The delivery catheter 926 for delivering the first fixation device 200 may be delivered further into the ventricle than the delivery catheter 904 for delivering the second fixation device 202. The delivery catheter 926 for delivering the first fixation device 200 may optionally therefore be longer than the delivery catheter 904 for delivering the second fixation device 202. Advantageously, the delivery catheter 926 may be 2.5 cm to 6 cm longer than the delivery catheter 904.

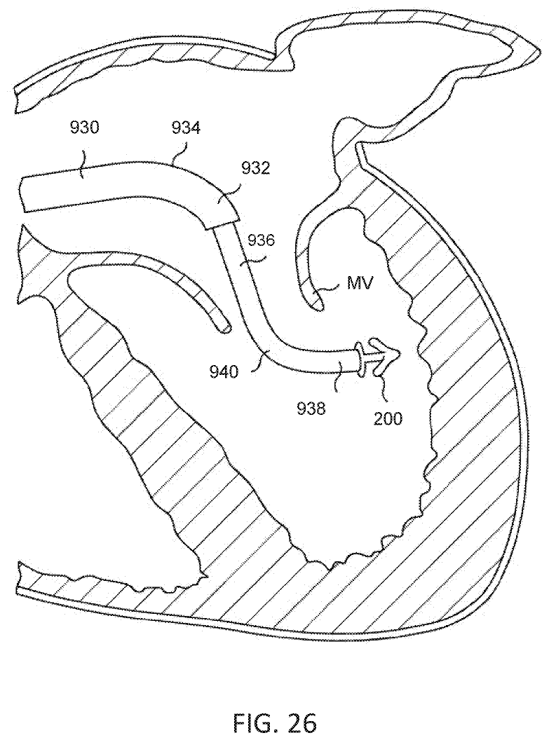

FIG. 26 shows schematically an alternative exemplary implementation, in which a fixation device delivery system is configured to be delivered to the ventricle for delivery of a first fixation device 200 thereto. In this implementation, the deflection of the delivery system in the ventricle is achieved by the delivery catheter 936. While FIG. 26 shows an example for delivery to the left ventricle, a corresponding delivery system may be implemented for delivery to the right ventricle, as will be appreciated.

The fixation device delivery system includes an inner guide catheter 930 and a delivery catheter 936. The inner guide catheter 930 is steerable to position a distal end 932 of the inner guide catheter 930 in a desired orientation within the left atrium. The inner guide catheter 930 has a lumen therethrough, through which the delivery catheter 936 may be delivered to the left atrium and then into the left ventricle. The delivery catheter 936 is releasably coupled to a first fixation device 200 which is to be used to engage and/or secure a pacemaker lead or leads into or adjacent a valve commissure, as described above. The releasable coupling is provided by a coupling member. The coupling member is deployable and retractable with the delivery catheter 936. The coupling member may be released from the fixation device 200 when the fixation device is deployed in a desired position, to maintain the lead(s) out of the way, at or adjacent an outer edge or annulus of the valve, such as in one of the valve's commissures.

The inner guide catheter 930 may be delivered to the left atrium through an outer guide catheter (not shown), in a similar manner as described in connection with FIG. 24 and delivery of the inner guide catheter 902 for the second fixation device 202. The inner guide catheter 930 is delivered through the lumen of the outer guide catheter, being guided therethrough to the left atrium, and out of the distal end of the outer guide catheter. At this point, the distal end 932 of the inner guide catheter 930 is not directed towards the mitral valve. In other words, the entry orientation at which the inner guide catheter 930 enters the left atrium, exiting the outer guide catheter, is not in line with, or parallel with, the inflow axis of the mitral valve. The entry orientation may be as much as substantially perpendicular, or at 90 °, to the inflow axis. The inner guide catheter 930 is provided with a steerable portion 934 which is configured to deflect the inner guide catheter in the left atrium from the entry orientation, not parallel to the inflow axis, to a second, inflow orientation, substantially parallel to the inflow axis. The steerable portion 934 is configured to deflect from a straight, or substantially straight, configuration to a bent configuration comprising a bend of up to 90 °. In this way, the inner guide catheter 930 may be re-oriented to be directed downwards and towards the mitral valve, parallel, or substantially parallel, to the inflow axis.

Similar to steerable portion 922 in FIG. 25, steerable portion 934 may be controlled by a respective steering control interface provided at or near the proximal end of the inner guide catheter 930. The steering control interface may include one or more respective knobs, levers, or other rotatable and/or translatable actuators to move the steerable portion 922, along with a respective wire assembly and a respective steerable ring, or the like, in a corresponding manner to that described above.

With the steerable portion 934 of the inner guide catheter 930 oriented substantially parallel to the inflow axis, the delivery catheter 936 is configured to pass through the mitral valve and into the left ventricle. The delivery catheter 936 is provided with a steerable portion 940. The steerable portion 940 is configured to deflect the delivery catheter 936 in the ventricle from the inflow orientation, substantially parallel to the inflow axis, to a third, delivery orientation, not parallel to the inflow axis. The steerable portion 940 is configured to deflect from a straight, or substantially straight, configuration to a bent configuration comprising a bend of up to 90 °. The third, delivery orientation of the delivery catheter 936 may be as much as substantially perpendicular, or at 90 °, to the inflow axis. In this way, the delivery catheter 936 may be re-oriented to be directed towards the heart tissue which the first fixation device 200 is configured to engage. The heart tissue may include one or more of a) edge portions of two native leaflets of the atrioventricular valve, where the edge portions are adjacent a valve commissure defined between the two native leaflets, b) one or more chordae tendineae, c) a papillary muscle, and/or d) a moderator band.

The steerable portion 940 may be controlled by a respective steering control interface provided at or near the proximal end of the delivery catheter 936. The steering control interface may include one or more respective knobs, levers, or other rotatable and/or translatable actuators to move the second steerable portion, along with a respective wire assembly and a respective steerable ring, or the like, in a corresponding manner to that described in connection with the steering control interface provided with the inner guide catheter 930.

Again, in view of the space limitations in the ventricle and/or to allow the delivery catheter 936 to be oriented as desired, the steerable portion 940 is preferably configured to make a sharp or tight turn. Optionally, the steerable portion 940 may be configured with a radius of curvature of 0.5 cm to 1 cm.

Additionally or alternatively, the steerable portion 940 may be disposed adjacent to the distal end 938 of the delivery catheter 936. Optionally, the steerable portion 940 may be configured 1 cm to 3 cm from the distal end 938 of the delivery catheter 936.

The delivery catheter 936 for delivering the first fixation device 200 may be delivered further into the ventricle than the delivery catheter 904 for delivering the second fixation device 202. The delivery catheter 936 for delivering the first fixation device 200 may optionally therefore be longer than the delivery catheter 904 for delivering the second fixation device 202. Advantageously, the delivery catheter 936 may be 2.5 cm to 6 cm longer than the delivery catheter 904.

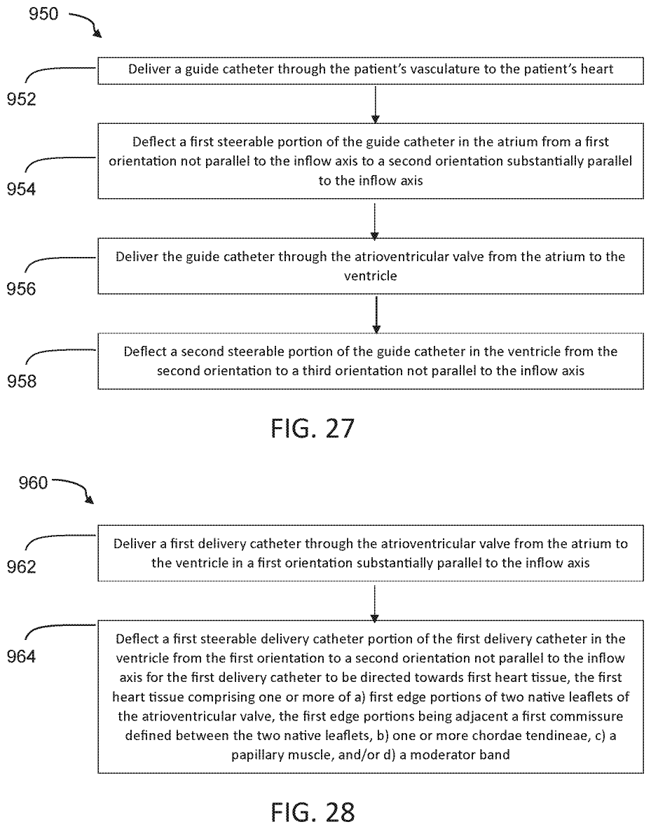

FIG. 27 illustrates a method 950 for delivering a transcatheter atrioventricular valve modification device to a delivery site within the heart of a patient. The method may be used to arrive at the configuration shown in FIG. 25. In step 952, a guide catheter is delivered through the patient's vasculature to the patient's heart. In step 954, a first steerable portion of the guide catheter is deflected in the atrium from a first orientation not parallel to the inflow axis to a second orientation substantially parallel to the inflow axis. In step 956, the guide catheter is delivered through the atrioventricular valve from the atrium to the ventricle. In step 958, a second steerable portion of the guide catheter is deflected in the ventricle from the second orientation to a third orientation not parallel to the inflow axis.

The guide catheter includes a lumen for passage therethrough of a delivery catheter for delivery of an atrioventricular valve modification device to the delivery site. The method 950 may further include delivering the first delivery catheter through the first lumen. Following delivery of the first delivery catheter through the lumen, and optional deployment of a first fixation device releasably coupled to the first delivery catheter, the first delivery catheter may be withdrawn from the lumen. Subsequently, a second delivery catheter may be delivered through the lumen, for optional deployment of a device releasably coupled to the second delivery catheter. Depending on the location for deployment of such device and/or the desired location or orientation to deliver the second delivery catheter, it may be desired to reposition the guide catheter before delivery of the second delivery catheter. Accordingly, after withdrawal of the first delivery catheter, the second steerable portion of the guide catheter may be deflected in the ventricle back to the second orientation before delivering the second delivery catheter through the lumen. The guide catheter may additionally be retracted or raised to move the distal end of the guide catheter back into the atrium for delivery of the second delivery catheter.

FIG. 28 illustrates a method 960 for delivering a transcatheter atrioventricular valve modification device to a delivery site within the heart of a patient. The method may be used to arrive at the configuration shown in FIG. 26. In step 962, a first delivery catheter is delivered through the atrioventricular valve from the atrium to the ventricle in a first orientation substantially parallel to the inflow axis. In step 964, a first steerable delivery catheter portion of the first delivery catheter is deflected in the ventricle from the first orientation to a second orientation not parallel to the inflow axis for the first delivery catheter to be directed towards first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

The method 960 may include the prior steps of delivering a guide catheter through the patient's vasculature to the atrium, and deflecting the guide catheter in the atrium from a third orientation not parallel to the inflow axis to a fourth orientation substantially parallel to the inflow axis for the guide catheter to deliver the first delivery catheter to the atrium in the first orientation.

The first delivery catheter may be releasably coupled to a first fixation device including first fixation elements. The method may further include engaging the first fixation elements with the first heart tissue. The method may additionally or alternatively include trapping a pacemaker lead or leads passing through the atrioventricular valve between the first commissure and the first fixation device. The method may additionally or alternatively include engaging the first fixation elements with a pacemaker lead or leads passing through the atrioventricular valve along with the one or more of b) the one or more chordae tendineae, c) the papillary muscle, and/or d) the moderator band, to secure the lead or leads of the pacemaker thereto.

The method may further include delivering a second delivery catheter through the atrioventricular valve from the atrium to the ventricle in the first orientation. The second delivery catheter may be releasably coupled to a second fixation device including second fixation elements. The method may further include engaging the second fixation elements with second heart tissue, the second heart tissue including second edge portions of two native leaflets of the native atrioventricular valve, the second edge portions being located away from the first commissure. In other words, the second edge portions may be further away from the first commissure than the first edge portions. This applies whether the second edge portions are on the same two leaflets as the first edge portions, as would be the case with a mitral valve, or on a different set of two leaflets, as may be the case with a tricuspid valve.

The method may alternatively include delivering a second delivery catheter to the atrium in the first orientation, the second delivery catheter being releasably coupled to a prosthetic atrioventricular valve. The method may further include deploying the prosthetic atrioventricular valve into the native atrioventricular valve so that the prosthetic atrioventricular valve expands into engagement with the native atrioventricular valve.

In any one or more of the embodiments described above and/or in connection with FIGS. 24-28, the first fixation device 200 and/or the second fixation device 202 may be as shown and/or described in connection with FIG. 3. Additional details of examples of a fixation device are provided in U.S. Patent Application Publication No. 2024/0382308, the disclosure of which is hereby incorporated by reference herein. For example, the first fixation device 200 for securing a pacemaker lead(s) L may take the same form as a second fixation device 202 for reducing atrioventricular valve regurgitation. Alternatively, the first fixation device 200 may be smaller than the second fixation device 202. The first fixation device 200 may be smaller than a known, commercially available fixation device. Such a known fixation device may be the MitraClip® G4 NT fixation device, available from Abbott Laboratories. The second fixation device 202 may be a known, commercially available fixation device. For example, the second fixation device 202 may be the MitraClip® G4 NT fixation device.

The first fixation device 200 may be smaller than the second fixation device 202 in any one or more of the ways described above in relation to FIGS. 15-23.

While the second delivery catheter has been described as being releasably coupled to a second fixation device for atrioventricular valve repair, instead, after deployment of a first fixation device for engaging and/or securing a pacemaker lead or leads, a prosthetic atrioventricular valve may be deployed for atrioventricular valve replacement, for example as described in relation to FIG. 12 and/or FIGS. 13-14.

As noted, some patients may have a pacemaker, or more than one pacemaker, fitted, with the or each pacemaker being provided with a lead, or more than one lead, which passes from an atrium of the heart to the corresponding ventricle. For example, one or more leads may pass from the right atrium to the right ventricle, passing through the tricuspid valve. By way of further example, one or more leads may pass from the left atrium to the left ventricle, passing through the mitral valve. The lead(s) may interfere with the proper placement and/or configuration of a fixation device; and/or may interfere with the proper placement and/or configuration of a transcatheter replacement valve. For example, the presence of a lead(s) may present a challenge to steering of a device to be deployed. In some cases, this may lead to a need to remove the lead(s) for the device deployment and then to replace the lead(s).

With reference to FIGS. 7A-C and 9-28 and accompanying description, a substantially permanent way of addressing the above problem is provided. A first fixation device 200 may be deployed to engage and/or secure the lead(s). The lead(s) may be trapped between a) a first fixation device engaging leaflet tissue adjacent a commissure defined between two leaflets, and b) the commissure so defined. Additionally, or alternatively, the lead(s) may be engaged by a first fixation device while also engaging heart tissue including one or more of a) one or more chordae tendineae, b) a papillary muscle, and/or c) a moderator band, so that the lead(s) and the heart tissue are secured together by the first fixation device.

In some implementations, where either a fixation device for reducing valve regurgitation or a prosthetic replacement valve for replacing the native valve is to be deployed, it may be advantageous to reposition the lead(s) temporarily before and/or during deployment of the fixation device or the prosthetic replacement valve. In this way, the lead(s) may be moved out of the way for delivery and/or deployment of the fixation device or the prosthetic replacement valve, without the step of deploying a first fixation device to permanently trap the lead(s). In other implementations, where a first fixation device to permanently trap the lead(s) is to be deployed, it may also be advantageous to reposition the lead(s) temporarily before and/or during deployment of the first fixation device. In this way, the lead(s) may be moved to a desired position for trapping by the first fixation device.

A transcatheter positioning device may therefore be provided for temporarily repositioning an object in the heart of a patient, such as a pacemaker lead or leads. The positioning device includes a positioning member having an undeployed configuration and a deployed configuration. The undeployed configuration is for delivery of the positioning device to an atrioventricular valve modification device delivery site in the heart of a patient. The deployed configuration is for the positioning member to engage and reposition an object in the heart. The object may include a lead or leads of a pacemaker passing through the atrioventricular valve from the atrium associated with the atrioventricular valve to the ventricle associated with the atrioventricular valve. The object may additionally or alternatively include one or more of a) one or more chordae tendineae, b) a papillary muscle, and/or c) a moderator band in the ventricle associated with the atrioventricular valve.

The object to be repositioned in the heart may be engaged by the positioning member in the deployed configuration. The positioning member may then be used to move and reposition the object. The object in the repositioned location may as a result no longer interfere with the delivery and/or deployment of an atrioventricular valve modification device, such as a fixation device or a prosthetic replacement valve, or any interference by the object may be reduced.

In one example, the positioning member is used to engage and reposition a pacemaker lead. The pacemaker lead may be initially located at or near an intended atrioventricular valve modification device delivery site, thereby potentially or actually interfering with the delivery and/or deployment of the device. The positioning member may be deployed to engage with the lead and to reposition the lead away from the intended device delivery site. For example, the lead may be repositioned to or towards a commissure defined between two native valve leaflets. An atrioventricular valve modification device may then be delivered and deployed to the valve without interference, or with reduced interference, from the pacemaker lead. After deployment of the valve modification device, the positioning member may be returned to its undeployed configuration and withdrawn from the heart.

The positioning device may be provided in a lumen, or more than one lumen, of a catheter. Advantageously, the catheter may be a catheter configured for another function, to which catheter the positioning device lumen(s) is added for the additional positioning functionality. For example, a positioning device lumen(s) and positioning device may be added to a delivery catheter used to deploy a fixation device or a prosthetic atrioventricular valve. Alternatively or additionally, a positioning device lumen(s) and positioning device may be added to an inner guide catheter used to guide a delivery catheter to a desired location in the heart. Alternatively or additionally still, a positioning device lumen(s) and positioning device may be added to an outer guide catheter used to guide a device delivery system, including an inner guide catheter, to a desired location in the heart.

Providing the positioning device in the delivery catheter and/or the outer guide catheter allows for use of existing steering mechanisms associated with the delivery catheter (for example, through the inner guide catheter delivering the delivery catheter) and/or the outer guide catheter. An advantage of providing the positioning device in the outer guide catheter would be that the positioning device could be used prior to advancing the atrioventricular valve modification device delivery system through the outer guide catheter. However, the outer guide catheter offers more limited steering capabilities compared with an atrioventricular valve modification device delivery system. An advantage of providing the positioning device in the delivery catheter would be more precise steering and optionally an additional degree of freedom using the control interface of an atrioventricular valve modification device delivery system. For example, the control interface may be configured to allow steering and/or adjustment in a +/−x-axis direction, in a +/−y-axis direction, and/or in a +/−z-axis direction. In an example delivery system, an outer guide catheter may be configured with a control interface providing S/L, septal/lateral, directional control and +/−height control. An inner delivery catheter may be configured with a control interface providing F/E, flex/extend, directional control.

In more detail, the catheter may be a delivery catheter configured to pass through the patient's vasculature to the delivery site. The delivery catheter includes a first lumen for delivery of an atrioventricular valve modification device to a delivery site in the heart. The atrioventricular valve modification device may be a fixation device or a prosthetic atrioventricular valve. The delivery catheter also includes a second lumen for the positioning device. The second lumen may be configured to constrain the positioning member of the positioning device within the second lumen in an undeployed configuration. The positioning member may be configured to be deployed from the second lumen to assume a deployed configuration.

Alternatively or additionally, a catheter provided for the positioning device may be an outer guide catheter configured to pass through the patient's vasculature towards the delivery site. The outer guide catheter includes a first lumen for delivery of an atrioventricular valve modification device delivery system to a delivery site in the heart. The atrioventricular valve modification device may be a fixation device or a prosthetic atrioventricular valve. The delivery system may include an inner guide catheter and/or a delivery catheter. The outer guide catheter also includes a second lumen for the positioning device. The second lumen may be configured to constrain the positioning member of the positioning device within the second lumen in an undeployed configuration. The positioning member may be configured to be deployed from the second lumen to assume a deployed configuration.

Alternatively or additionally still, a catheter provided for the positioning device may be an inner guide catheter configured to pass through the patient's vasculature towards the delivery site. The inner guide catheter includes a first lumen for delivery of a delivery catheter releasably coupled to an atrioventricular valve modification device to a delivery site in the heart. The atrioventricular valve modification device may be a fixation device or a prosthetic atrioventricular valve. The inner guide catheter also includes a second lumen for the positioning device. The second lumen may be configured to constrain the positioning member of the positioning device within the second lumen in an undeployed configuration. The positioning member may be configured to be deployed from the second lumen to assume a deployed configuration.

In each or any of the above examples, the first lumen of the respective catheter may be a main lumen of the catheter. The first lumen may be a central lumen, located substantially centrally in the catheter. The first lumen may have a larger diameter than other lumens in the catheter.

In each or any of the above examples, the second lumen of the respective catheter may be a secondary lumen of the catheter. The second lumen may be an off-center lumen, located outwards, for example radially away from, the center of the catheter. The second lumen may have a smaller diameter than the first lumen. The catheter may include multiple secondary lumens and more than one secondary lumen may be provided for housing one or more than one positioning device. One configuration of a catheter may include a polymer-extruded member having a main lumen and one or more than one secondary lumen.

The positioning member may be configured to take on a given shape or form when in the deployed configuration. The given shape or form is advantageously predetermined to provide a structure which may engage a pacemaker lead, to facilitate movement or repositioning of the lead by the positioning member. The given shape or form may include a receiving zone into which a pacemaker lead may be received or guided to assist in moving and repositioning the lead. For example, the receiving zone my include one or more of a curve, indent, recess, hook, mouth, and/or arms. The given shape or form may alternatively or additionally include a push zone which may engage and reposition a pacemaker lead, optionally without accommodating the lead within its structure. For example, the push zone may include one or more of an outward curve, a loop, a hook, a straight portion, a blocking portion, and/or a bumper portion.

The positioning member may be configured to take on the given shape or form when unconstrained. In other words, the deployed configuration of the positioning member may be a relaxed or unstressed configuration of the positioning member. The positioning member may be forced out of the relaxed or unstressed configuration in the undeployed configuration. For example, the positioning member may be held under tension in the undeployed configuration. The positioning member may be additionally or alternatively be constrained by being held or stored in one or more than one second lumen of the catheter.

In any or each of the above configurations, the positioning member may include a shape-memory material. Optionally, the positioning device as well as the positioning member may include a shape-memory material. The shape-memory material may be provided in the form of an elongate member. For example, the shape-memory material may be provided in the form of a wire. The shape-memory material may be configured to assume the deployed configuration when the positioning member is unconstrained. The shape-memory material may include a shape-memory alloy, such as a nickel-titanium alloy. In particular, the shape-memory material may be Nitinol. Alternatively or additionally, the shape-memory material may include a shape-memory polymer. Advantageously, the positioning member, optionally also the positioning device, is provided in the form of a Nitinol wire.

Deployment and retraction of the positioning device may be controlled by a positioning device control interface. The positioning device control interface may be provided at a proximal end of the catheter housing the positioning device. The control interface may include a positioning device control mechanism for deploying the positioning member between the undeployed configuration and the deployed configuration. The control interface may include one or more than one knob, lever, or other rotatable and/or translatable actuator to move the positioning member between the undeployed and deployed configurations. The positioning member may be configured to be retracted back into the second lumen for withdrawal from the heart of the patient with the delivery system.

It may be advantageous in some implementations to provide for rotation of the positioning member. The positioning device control mechanism may additionally include a rotational control mechanism for rotating the positioning member.

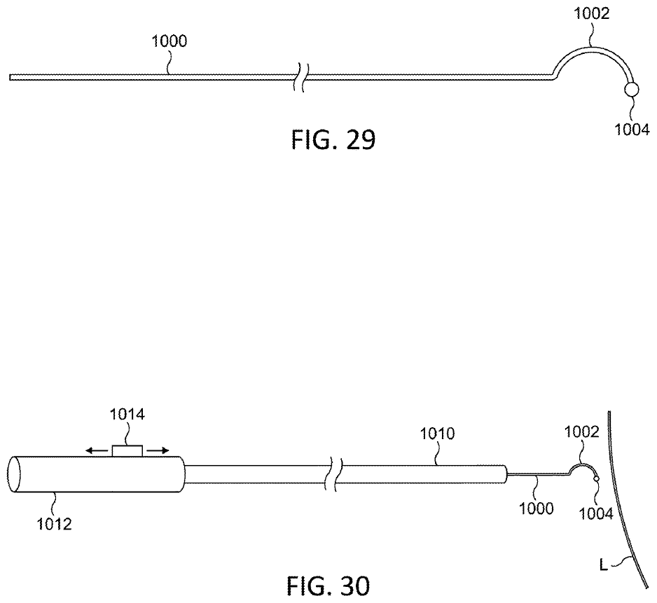

As discussed, the positioning member may take on a number of different shapes or forms in the deployed configuration. Some example shapes or forms are described with reference to FIGS. 29-48. Any one, or any combination, of these shapes or forms may be implemented in a positioning device. A single positioning member may include one or a combination of shapes or forms in the deployed configuration. In implementations with more than one positioning member, each positioning member may include one or a combination of shapes or forms in the deployed configuration. Each positioning member may have the same structure as the other positioning member(s) of the positioning device. Alternatively, each positioning member may have a different structure from the other positioning member(s).

FIG. 29 shows a positioning device 1000, including a positioning member 1002 in a deployed configuration including a hook shaped portion. The positioning member 1002 includes a curved distal end. Optionally, the positioning member 1002 may include an atraumatic tip 1004 to the distal end, for example by being smoothed, rounded, and/or provided with a bulbous tip. The hook shaped portion provides two functional sides. The concave side of the hook shaped portion offers a receiving zone, which may capture the pacemaker lead within the hook. The size of the curved portion determines the degree of tolerance in the hook shaped portion engaging the lead. The positioning member may be deployed first and then moved to engage the pacemaker lead. Alternatively, the positioning member may be deployed where the lead is initially located, so that the positioning member ensnares or wraps around, partially or fully, the lead while the positioning member is being deployed. Once engaged, the lead may be repositioned by moving the positioning member to the desired location for the lead. The convex side of the hook shaped portion offers a push zone, which may be urged against the lead. Again, once engaged, the lead may be repositioned by moving the positioning member to the desired location for the lead. The positioning member in this configuration provides for atraumatic engagement with a pacemaker lead and any part of the heart anatomy the positioning member may come into contact with during repositioning of the lead.

FIG. 30 schematically shows the positioning device 1000 of FIG. 29 being provided in a lumen 1008 of a catheter 1010. A positioning device control interface 1012 is provided at a proximal end of the catheter 1010. The positioning device control interface 1012 is provided with an actuator 1014 for controlling the deployment and retraction of the positioning device 1000. The actuator 1014 here is shown as a translatable slide button, but the actuator may include one or more than one knob, lever, or other rotatable and/or translatable actuator to move the positioning member between the undeployed and deployed configurations. Actuation of the actuator 1014 in a first direction is configured to deploy the positioning device 1000 from the end of the catheter 1010. Actuation of the actuator 1014 in a second, reverse or opposite direction is configured to retract the positioning device 1000 into the catheter 1010, to return the positioning member to its undeployed configuration. The positioning device control interface may additionally include a rotational control mechanism for rotating the positioning member about the longitudinal axis of the catheter 1010. This may assist with engaging and/or repositioning the pacemaker lead.

Once a pacemaker lead has been engaged, repositioning the lead may be achieved by further deployment of the positioning device to move the lead in the direction of deployment of the positioning device. The positioning device may be configured to continually or discretely change its orientation as it is further deployed from the catheter, to reposition the lead laterally and/or longitudinally in a predetermined manner. Alternatively, the positioning device may simply be extended longitudinally out of the catheter to reposition the lead. In addition or alternatively, the catheter itself may be configured to be steerable and/or a guide catheter through which the catheter passes may be configured to be steerable. Using one or both such steerable catheters, the orientation of the positioning device may be adjusted so that the positioning member repositions the lead to or towards a desired location.

The above discussion in relation to FIG. 30 may be correspondingly applied to any and each of the positioning devices described herein, whatever their deployed shape or form.

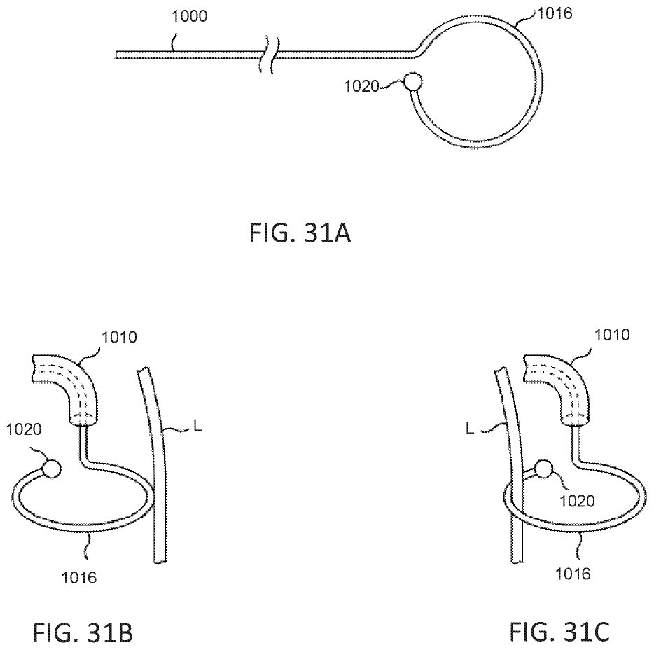

FIGS. 31A-C show a positioning device 1000, including a positioning member 1016 in a deployed configuration including a loop shaped portion. The positioning member 1016 includes a hoop or loop at its distal end 1018. Optionally, the positioning member 1016 may include an atraumatic tip 1020 to the distal end 1018, for example by being smoothed, rounded, and/or provided with a bulbous tip. The loop shaped portion may provide two functional sides. The inside of the loop, or the concave side of the loop shaped portion, offers a receiving zone, which may capture the pacemaker lead within the loop, as shown in FIG. 31C. The loop may include an opening in the turn of the loop, the opening allowing the lead to pass into the inside of the loop. The opening may be defined by the separation between the positioning member 1016 at the start of the loop shaped portion and the distal end 1018 of the positioning member. The size of the opening may influence the degree of tolerance in the loop shaped portion engaging the lead. The positioning member may be deployed first and then moved to engage the pacemaker lead. Alternatively, the positioning member may be deployed where the lead is initially located, so that the positioning member ensnares or loops around the lead while the positioning member is being deployed. Once engaged, the lead may be repositioned by moving the positioning member to the desired location for the lead. The convex or outer side of the loop shaped portion offers a push zone, which may be urged against the lead, as shown in FIG. 31B. Again, once engaged, the lead may be repositioned by moving the positioning member to the desired location for the lead. The positioning member in this configuration provides for atraumatic engagement with a pacemaker lead and any part of the heart anatomy the positioning member may come into contact with during repositioning of the lead. The loop shape portion may be generally in the same plane as the distal end of the catheter lumen from which the positioning member is deployed, as shown in FIG. 31A. Alternatively, the loop shape portion may assume an angle relative to the plane of the distal end of the catheter lumen from which the positioning member is deployed, as shown in FIGS. 31B-C. The loop shape portion may be perpendicular to the part of the positioning device extending into the lumen. This may provide a lasso shape to the positioning member.

The positioning device 1000 may optionally be provided with a positioning member which in a deployed configuration includes a double loop shaped portion. One or other of the loops may engage a pacemaker lead. This may be within one of the loops or by pushing a lead with the loop. An intersection between adjacent loops, for example a U or V shaped intersection, may be used to provide a receiving zone for capturing and engaging a lead. If there is more than one pacemaker lead, each loop may engage a respective lead. The positioning member in this configuration provides for atraumatic engagement with a pacemaker lead and any part of the heart anatomy the positioning member may come into contact with during repositioning of the lead.

Such a U or V shaped zone may alternatively be provided by a positioning member which in a deployed configuration forms a U or V shaped portion, for example by including a substantially heart shaped portion. The ‘top’ of the heart shaped portion may provide the U or V shaped zone for receiving a pacemaker lead. The positioning member in this configuration may provide for atraumatic engagement with a pacemaker lead and any part of the heart anatomy the positioning member may come into contact with during repositioning of the lead.

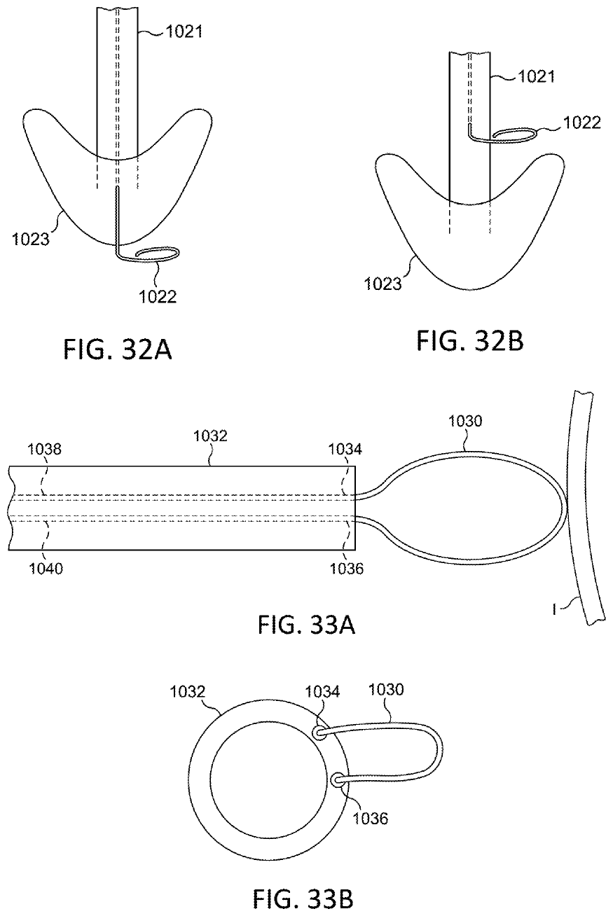

FIGS. 32A-B show a positioning device 1000, including a positioning member 1022 in a deployed configuration including a coil or helix shaped portion. The positioning member 1022 includes a coiled member with one or more than one turn at its distal end. Optionally, the positioning member 1022 may include an atraumatic tip to the distal end, for example by being smoothed, rounded, and/or provided with a bulbous tip. In implementations where the distal end terminates radially inwards of the turn(s) forming the coil or helix shaped portion, the outermost part of the positioning member 1022, being a curved coil or helix, will be atraumatic. The coil or helix shaped portion may provide two functional sides. The inside of the coil or helix offers a receiving zone, which may capture the pacemaker lead within the coil or helix. The positioning member may be deployed where the lead is initially located, so that the positioning member ensnares or coils around the lead while the positioning member is being deployed. Once engaged, the lead may be repositioned by moving the positioning member to the desired location for the lead. The convex or outer side of the coil or helix shaped portion offers a push zone, which may be urged against the lead. Again, once engaged, the lead may be repositioned by moving the positioning member to the desired location for the lead. The positioning member in this configuration provides for atraumatic engagement with a pacemaker lead and any part of the heart anatomy the positioning member may come into contact with during repositioning of the lead.

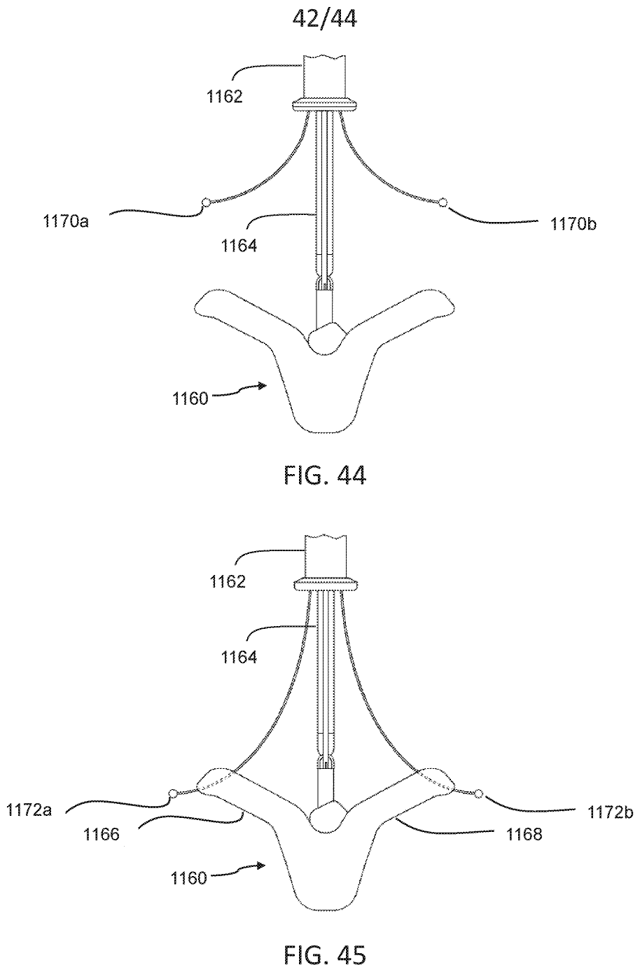

The positioning device 1000 in FIG. 32A is shown being deployed from a portion of a fixation device 1023. The portion may be a side of the main body of the fixation device, as shown, Alternatively, the portion may be a side of a fixation element of a fixation device, or an outer or inner end of a fixation element of a fixation device. The positioning device 1000 in FIG. 32B is shown being deployed from a delivery catheter 1021 for delivering a fixation device 1023 to an atrioventricular valve. However, the positioning device 1000, in this arrangement and in the other arrangements described herein, may be provided with any catheter of a delivery system, including any one or more of a delivery catheter, an inner guide catheter, and/or an outer guide catheter. The delivery system may be configured for delivery of a fixation device and/or a prosthetic replacement valve. Reference is made to FIGS. 44-47 and the associated description for exemplary structural and/or configurational arrangements which may be implemented with positioning device 1000.

An alternative to a coil or helix shaped portion for the positioning member is a spiral shaped portion. Deploying such a spiral shaped portion where a pacemaker lead is initially located may allow the spiral shaped portion to gradually wrap around the lead, with smaller turn(s) of the turns of the spiral capturing the lead more securely for repositioning. Optionally but advantageously, a radius of curvature of the spiral shaped portion may decrease as the positioning member is deployed. In this way, a larger-dimension turn may be deployed first, to provide a larger capture zone for the lead, then a smaller-dimension turn may be deployed to more securely hold the lead for repositioning. Alternatively, the outside structure of the positioning member may be used to push the lead to a desired location.

Other forms and/or shapes for the positioning member may be provided. For example, the positioning member in the deployed configuration may include an S shaped portion. Alternatively, the positioning member in the deployed configuration may include a FIG. 3 shaped portion. Alternatively, the positioning member in the deployed configuration may include a FIG. 8 shaped portion.

The positioning member may be arranged at the distal end of the positioning device 1000. In this arrangement, the positioning member may be housed or stowed in a lumen of a catheter in the undeployed configuration. By urging the distal end of the positioning device 1000 out of the lumen, the positioning member may take on its deployed configuration. The positioning device 1000 may be moved in and out of the lumen by a control interface at the proximal end of the catheter, actuating a proximal end of the positioning device.

In an alternative arrangement, both ends of the positioning device 1000 may remain within the catheter. An intermediate portion of the positioning device 1000 may provide the positioning member, which may be deployed from the catheter. The intermediate portion may be deployed by urging one or both of the ends of the positioning device 1000 towards the distal end of the catheter. The intermediate portion may then be unconstrained and take on the deployed configuration of the positioning member. In one arrangement, both ends of the positioning device 1000 are located at the proximal end of the catheter. When both ends are in their proximal-most position, the positioning member, provided by the intermediate portion, is constrained in its undeployed configuration. In particular, the positioning member is held under tension in this arrangement. Actuating one or both of the proximal ends of the positioning device 1000 to move the end or ends distally reduces the tension on the positioning member and allows the positioning member to take on its deployed configuration, outside of the catheter. For this arrangement, one lumen in the catheter may be provided for both ends of the positioning device 1000 to pass through. Alternatively, two lumens may be provided, one for a first end of the positioning device and the other for a second end of the positioning device. In either case, the intermediate portion of the positioning device is located at the distal end of the catheter so that the positioning member may be deployed from the catheter when one or both ends of the positioning device are moved distally. This arrangement offers an atraumatic positioning member in that the ends of the positioning device are retained within the catheter during use.

In an alternative arrangement, both ends of the positioning device 1000 are not located at the proximal end of the catheter. A first end may be so located, while the second end of the positioning device may be located at a location more distal along the catheter. Indeed, the second end may be located at or adjacent the distal end of the catheter. In this case, the intermediate portion providing the positioning member may be provided adjacent to the second end, for deployment out of the catheter. The second end may be fixed in position and the first end may be actuated to deploy the positioning device.

FIGS. 33A-B show an arrangement in which a positioning member 1030 is provided by an intermediate portion of the positioning device 1000. FIG. 33A shows a schematic side view of a catheter 1032 and positioning device 1000, with the positioning member 1030 in a deployed configuration. FIG. 33B shows a schematic end view of the catheter 1032 with the positioning member 1030 in the deployed configuration. The catheter 1032 is provided with two secondary lumens 1034, 1036. A first end 1038 of the positioning device 1000 passes through a first lumen 1034. A second end 1040 of the positioning device 1000 passes through a second lumen 1036. In the undeployed configuration of the positioning member 1030, both ends 1038, 1040 of the positioning device 1000 are under tension, for example by being retracted within the lumens 1034, 1036. In this configuration, only a part of the intermediate portion is located outside of the lumens 1034, 1036. That part runs between the distal ends of the first and second lumens 1034, 1036 at the end of the catheter 1032. The positioning member 1030 is deployed from the catheter 1032 by releasing the tension on the first and/or second end 1038, 1040 of the positioning device 1000. One or both of the first and/or second end 1038, 1040 is actuated to move distally. This allows the positioning member 1030, provided by the intermediate portion of the positioning device, to be deployed from the distal end of the catheter 1032. In the example shown in FIGS. 33A-B, the positioning member 1030 forms a loop shaped portion in the deployed configuration. The deployed positioning member may then be used to push a pacemaker lead to reposition the lead to a desired location. It will be appreciated that the positioning member may alternatively take on any of the forms and/or shapes described herein, among other alternatives. Reference is made to FIGS. 44-47 and the associated description for exemplary structural and/or configurational arrangements which may be implemented with the positioning device.

The positioning device 1000 may be provided by a single component, such as one wire. In some arrangements, the positioning device 1000 may include two positioning members which may cooperate with each other to engage and reposition a pacemaker lead.

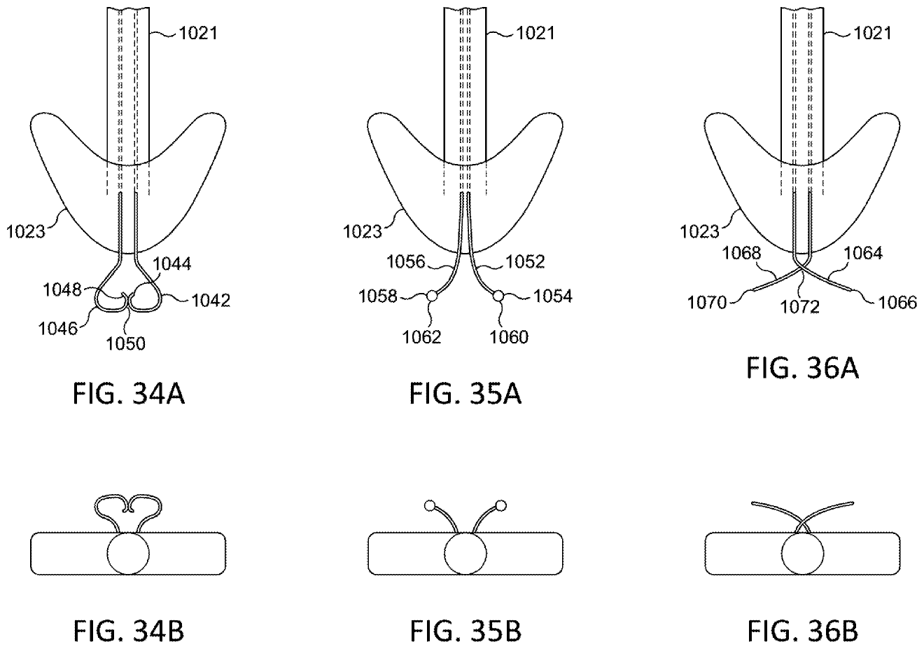

FIGS. 34A-B show schematically a side view and a top-down view of a positioning device 1000, including a first positioning member 1042 having a first distal end 1044 and a second positioning member 1046 having a second distal end 1048. In a deployed configuration, the first and second positioning members 1042, 1044 have the same form and shape. The first and second positioning members 1042, 1044 are configured to be deployed opposite to each other to provide a symmetrical shape in combination. The distal ends 1044, 1048 curve proximally, back towards the catheter, and meet at an intersection 1050. As described above, the intersection 1050 may receive and push a pacemaker lead to a desired location. Alternatively, the lead may be captured within the interior space defined by the positioning members 1042, 1046 by deploying the positioning members where the lead is initially located so that the lead is captured during the deployment process. In either case, this arrangement offers an atraumatic positioning device in that the outer structure of the deployed positioning members is curved. As before, optionally, the distal ends of the positioning members may be additionally configured to be atraumatic. The first and second positioning members may be deployed independently of each other. The first and second positioning members may be deployed one after the other. Alternatively, the first and second positioning members may be deployed simultaneously. Reference is made to FIGS. 44-47 and the associated description for exemplary structural and/or configurational arrangements which may be implemented with the positioning device.

FIGS. 35A-B show schematically a side view and a top-down view of a positioning device 1000, including a first positioning member 1052 having a first distal end 1054 and a second positioning member 1056 having a second distal end 1058. A first magnet 1060 is provided at the first distal end 1054 and a second magnet 1062 is provided at the second distal end 1058. The magnets 1060, 1062 are configured to be attracted to each other to releasably lock together. FIGS. 35A-B show a stage after deployment of the positioning members but before the magnets 1060, 1062 have locked together. The magnets 1060, 1062 are configured to be released from each other by moving one of the positioning members 1052, 1056 relative to the other of the positioning members. A pacemaker lead may be captured between the first and second positioning members 1052, 1056 and held by the locking together of the first and second magnets 1060, 1062. The first and second positioning members 1052, 1056 may be deployed one after the other, so that the magnets do not lock together before the lead has been received between the positioning members. After the lead has been repositioned and any other atrioventricular valve modification procedure has been carried out, the positioning members may be retracted one at a time. In this way, the locking between the magnets may be released at the repositioned location of the lead, avoiding potentially pulling the lead back towards the catheter during retraction of the positioning device. Reference is made to FIGS. 44-47 and the associated description for exemplary structural and/or configurational arrangements which may be implemented with the positioning device.

FIGS. 36A-B show schematically a side view and a top-down view of a positioning device 1000, including a first positioning member 1064 having a first distal end 1066 and a second positioning member 1068 having a second distal end 1070. In a deployed configuration, the first distal end 1066 is configured to curve away from first the lumen the first distal end is deployed from in a first direction towards the second lumen the second distal end 1070 is deployed from. Correspondingly, the second distal end 1070 is configured to curve away from the second lumen the second distal end is deployed from in a second direction towards the first lumen the first distal end 1066 is deployed from. In this way, the positioning members 1064, 1068 are configured to cross each other at an intersection 1072. The outward-facing side of the intersection 1072 may provide a receiving zone to engage a pacemaker lead and to push the lead to a desired location. The intersecting positioning members may in the deployed configuration have the form of a wishbone shaped portion. Optionally, the distal ends of the positioning members may include an atraumatic tip, as described above. Reference is made to FIGS. 44-47 and the associated description for exemplary structural and/or configurational arrangements which may be implemented with the positioning device.

In alternative arrangements, two positioning members may be deployed to cooperate with each other and form any of the shaped portions described herein. For example, two positioning members may be deployed to form a combined portion having a shape including a hook shape, a loop shape, a double loop shape, a coil shape, a helix shape, a spiral shape, an S shape, a FIG. 3 shape, a FIG. 8 shape, a U shape, a V shape, an X shape, and/or a Y shape, among other shapes.

The positioning devices 1000 described herein are intended to be used in an atrioventricular valve modification device delivery system. The system may be configured to deliver an atrioventricular valve modification device to a delivery site in the heart while the positioning device is in the deployed configuration. In other words, while a pacemaker lead is at least temporarily repositioned, an atrioventricular valve modification device may be delivered and deployed.

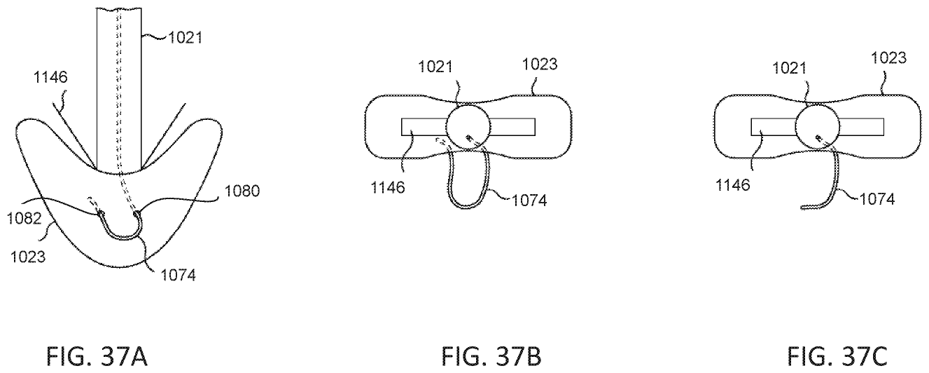

FIGS. 37A-C show a system in which a positioning device 1000 and a fixation device 1023 cooperate with each other. The positioning device 1000 is configured to pass through an opening 1080 in the fixation device 1023 in the deployed configuration of the positioning device. The opening 1080 may be located in the main body of the fixation device 1023. The opening 1080 may be located on a distal side of the main body. Additionally or alternatively, the opening 1080 may be provided in an optional covering on the fixation device 1023. In this way, the positioning member 1074 may be deployed through the opening 1080, to take on any of the positioning member forms and/or shapes described herein, for engaging and repositioning a pacemaker lead. The fixation device 1023 may then be deployed while the lead is repositioned. Following deployment of the fixation device, the positioning member may be retracted to its undeployed configuration, passing back through the opening 1080. The positioning member may be fully withdrawn from the fixation device, into a lumen of the catheter housing the positioning device. As shown in FIGS. 37A-C, the fixation device 1023 may be provided with a second opening 1082. The second opening 1082 may be adjacent the first opening 1080. During deployment of the positioning member, the distal end of the positioning member may be configured to pass into and through the second opening 1082, leaving the positioning member deployed while the distal end is temporarily held in the fixation device 1023 through the second opening 1082. Alternatively, the distal end of the positioning member may be located through the second opening 1082 before deployment of the positioning member, to help facilitate positioning of the positioning member during deployment, as the positioning member is deployed through the first opening 1080. Reference is made to FIGS. 44-47 and the associated description for exemplary structural and/or configurational arrangements which may be implemented with the positioning device.

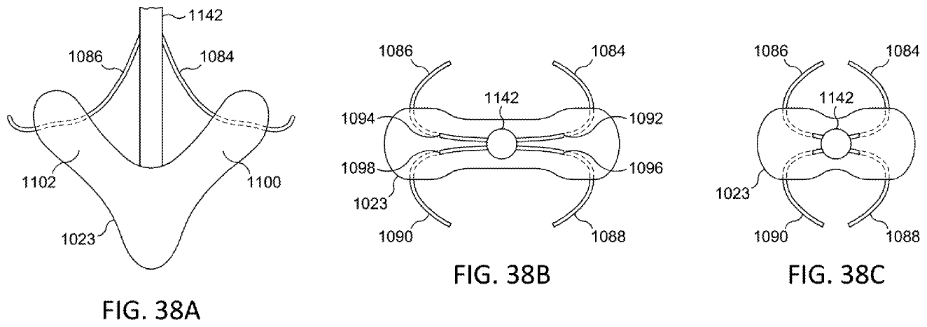

FIGS. 38A-C show another system in which a positioning device 1000 and a fixation device 1023 cooperate with each other. FIG. 38A shows schematically a side view of the system. FIG. 38B shows schematically a top view of the system, with the fixation elements of the fixation device open. FIG. 38C shows schematically a top view of the system, with the fixation elements of the fixation device closed. Reference is made to FIGS. 44-47 and the associated description for exemplary structural and/or configurational arrangements which may be implemented with the positioning device.

In this system, the positioning device 1000 is provided with four positioning members 1084, 1086, 1088, 1090. Each positioning member may be provided in a respective lumen of the catheter housing the positioning device, or may share one or more lumen. A first positioning member 1084 is configured to pass through a first opening 1092 on a first side of a first fixation element 1100 of the fixation device 1023. A second positioning member 1086 is configured to pass through a second opening 1094 on the first side of a second fixation element 1102 of the fixation device 1023. A third positioning member 1088 is configured to pass through a third opening 1096 on a second side of the first fixation element 1100. A fourth positioning member 1090 is configured to pass through a fourth opening 1098 on the second side of the second fixation element 1102. The openings may be provided through a respective structural component of the fixation elements and/or through an optional covering provided on the fixation elements. The first and second positioning members 1084, 1086 are configured to extend through the respective first and second openings 1092, 1094 on the first side of the fixation device and to extend outwards, away from the fixation device, and towards each other. In the open configuration of the fixation elements, the first and second positioning members are separated from each other and define a receiving zone for receiving a pacemaker lead on the first side of the fixation device. In the closed configuration of the fixation elements, the first and second positioning members are positioned closer to each other, optionally closing off the receiving zone in a pincer-like manner, to hold a pacemaker lead inside the closed positioning members. The lead may thereby be held out of the way of the fixation elements which may then be maneuvered and/or deployed at a desired delivery site.

On the other side of the fixation device, the third and fourth positioning members 1088, 1090 are configured to extend through the respective third and fourth openings 1096, 1098 on the second side of the fixation device and to extend outwards, away from the fixation device, and towards each other. In the open configuration of the fixation elements, the third and fourth positioning members are separated from each other and define a second receiving zone for receiving a pacemaker lead on the second side of the fixation device. In the closed configuration of the fixation elements, the third and fourth positioning members are positioned closer to each other, optionally closing off the second receiving zone in a pincer-like manner, to hold a pacemaker lead inside the closed positioning members. The lead may thereby be held out of the way of the fixation elements which may then be maneuvered and/or deployed at a desired delivery site.

In this way, the fixation device and positioning device may engage and reposition a pacemaker lead, no matter which side of the fixation device a pacemaker lead happens to be initially located.

To assist in capturing a lead, one or both of the positioning members on any one side may be extended further from the respective opening. The positioning member(s) may be extended until the positioning members close off the receiving zone by their distal ends engaging each other or intersecting or overlapping.

In alternative arrangements, only the first and second positioning members on the first side of the fixation device may be provided. Alternatively, only the third and fourth positioning members on the second side of the fixation device may be provided.

The positioning members may be fully retracted from the fixation device. The retraction may take place independently or separately for each positioning member. Alternatively, the positioning members may be retracted simultaneously.

In alternative arrangements, the positioning members may be configured to be left behind with the fixation device. The positioning members may be configured to be separable from the positioning device to be left behind. This may be implemented using a locking and release mechanism on each positioning member. Alternatively, each positioning member may be frangibly coupled with the positioning device, with the coupling being breakable upon applying tension to the proximal end of the positioning device. The positioning members may optionally be formed of a bioabsorbable material, which may be broken down in the body over time, for example through biosorption. The bioabsorbable material may be formed from, or include, one or a combination of polylactic acid (PLA) and/or poly(lactic-co-glycolic) acid (PLGA).

As noted above, the positioning device may be provided in a lumen, or more than one lumen, of a catheter. The catheter may be a catheter configured for another function, to which catheter the positioning device lumen(s) is added for the additional positioning functionality. For example, a positioning device lumen(s) and positioning device may be added to an outer guide catheter used to guide a device delivery system to a desired location in the heart. The device delivery system may include a delivery catheter and optionally further include an inner guide catheter for delivering the delivery catheter. The delivery catheter may be configured to deliver an atrioventricular valve modification device, which may be a fixation device or a prosthetic atrioventricular valve.

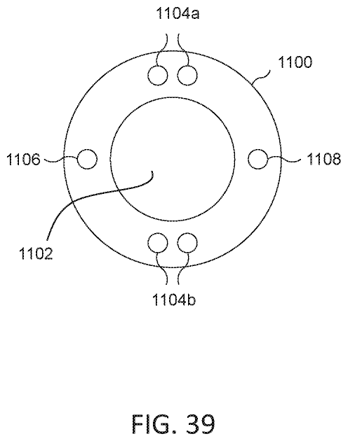

FIG. 39 shows a cross-sectional view of an outer guide catheter 1100 configured to pass through the patient's vasculature towards a delivery site in the heart. The outer guide catheter 1100 may be formed by a polymer-extrusion process. The outer guide catheter 1100 includes a first, main lumen 1102 for delivery of an atrioventricular valve modification device delivery system to a delivery site in the heart. The outer guide catheter also includes a number of secondary lumens formed within a circumferential wall of the outer guide catheter. A first set of secondary lumens 1104 is provided for steering the outer guide catheter 1100. In the arrangement shown in FIG. 39, there are four steering lumens 1104. A respective steering wire or cable is provided in each of the steering lumens 1104. The steering wires couple to a steerable member, optionally a ring member, located in the catheter where steerability of the catheter is desired. The steering lumens 1104 may be spaced with regular spacing between them around the catheter, or, as shown, pairs of the lumens 1104a, b may be located more closely together. Providing four steering lumens allows the outer guide catheter 1100 to be steered in any direction. In some arrangements, however, there may be two steering lumens, arranged diametrically opposite each other.

The outer guide catheter 1100 is also provided with a positioning device secondary lumen 1106. The positioning device lumen 1106 is configured to house the positioning device. The positioning device lumen 1106 is configured to allow the positioning member of the positioning device to be deployed from the distal end of the lumen to assume a deployed configuration. The positioning device lumen 1106 may be configured to constrain the positioning member of the positioning device within the lumen in an undeployed configuration.

The outer guide catheter 1100 is also provided with an additional secondary lumen 1108. The lumen 1108 is optional. The lumen 1108 may be employed for an additional functionality. Alternatively, the lumen 1108 may be provided with a further positioning device, to allow for a positioning member to be deployed from either side of the outer guide catheter. Further positioning device lumens may be provided in the outer guide catheter. For example, the catheter may include one positioning device lumen, two positioning device lumens, three positioning device lumens, four positioning device lumens, or more than four positioning device lumens. A pair of positioning device lumens may be provided adjacent one another in the catheter. This may allow for two positioning members to be deployed to cooperate with each other. This may alternatively allow for one positioning member to be deployed from a first of the lumens and to pass into the second of the lumens. In some arrangements, the ends of the positioning device are located in a respective lumen of the lumen pair and an intermediate portion of the positioning device is deployed from the catheter by extending the positioning member from one or both of the lumens in the pair.

As noted, a positioning device lumen(s) and positioning device may alternatively, or additionally, be added to a delivery catheter used to deliver an atrioventricular valve modification device to a desired location in the heart. The atrioventricular valve modification device may be a fixation device or a prosthetic atrioventricular valve.

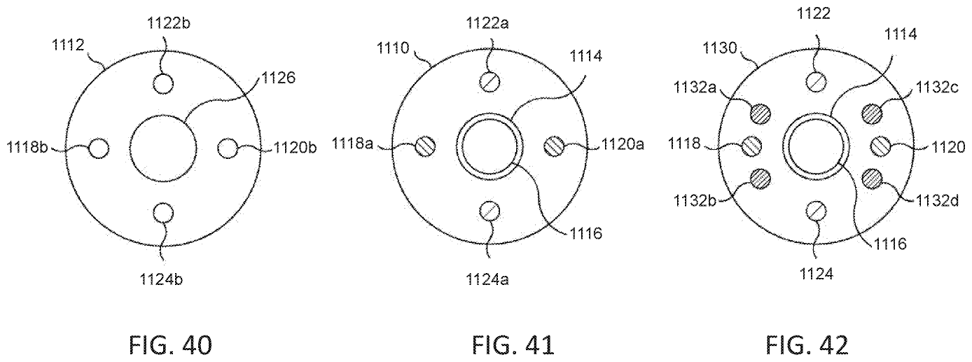

FIGS. 40-41 show cross-sectional views of a delivery catheter 1110 configured to pass through the patient's vasculature towards a delivery site in the heart. The delivery catheter 1110 is shown without a positioning device. The delivery catheter 1110 shown in FIGS. 40-41 may represent a known and/or commercially available delivery catheter. FIG. 40 illustrates a cross-sectional view of a distal end of the delivery catheter 1110 on which an end member 1112 has been fitted. FIG. 41 illustrates a cross-sectional view of the delivery catheter 1110 adjacent the distal end of the catheter.

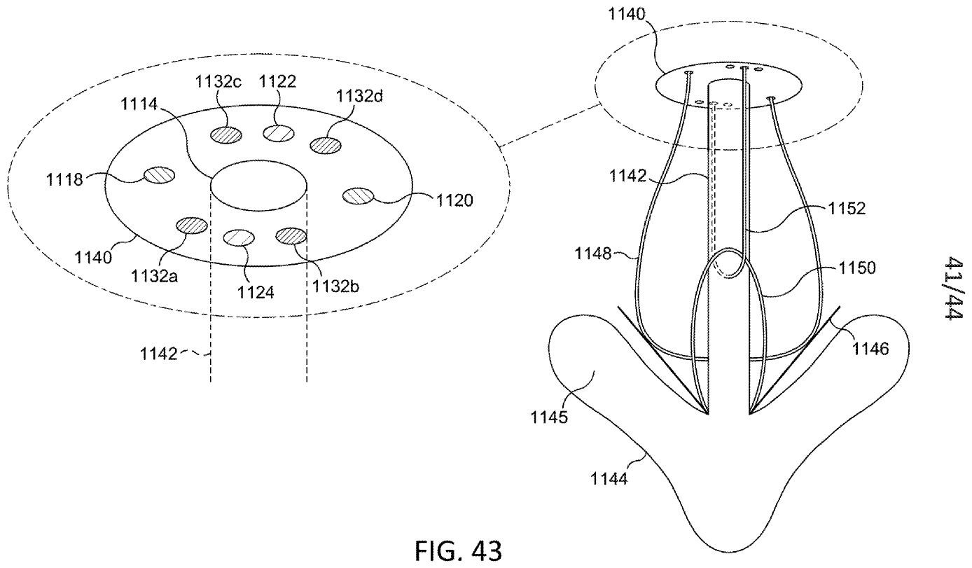

The delivery catheter 1110 includes a first, main lumen 1114. The main lumen 1114 is centrally located in the catheter. An actuatable member 1116, such as a rod or mandrel, extends through the main lumen 1114 and is extendable and retractable from the distal end of the delivery catheter. The actuatable member 1116 may be releasably coupled to a fixation device 1144 (shown schematically in FIG. 43) to actuate the fixation elements 1144 of the fixation device during deployment.

The delivery catheter 1110 includes two secondary lumens 1118a, 1120a, provided off-center, in a circumferential wall of the catheter. The secondary lumens 1118a, 1120a are provided with a gripper line or gripper lines 1148 which couple with respective gripping elements 1146 on the fixation device 1144. A gripper line 1148 may be configured to actuate a gripping element 1146 to raise or lower the gripping element during deployment of the fixation device 1144. Gripping elements 1146, along with the associated lumens 1118, 1120 and gripper lines 1148, are optional, but advantageous in facilitating grasping of heart tissue, especially valve leaflets, in combination with the fixation elements 1145.

The delivery catheter 1110 includes two further secondary lumens 1122a, 1124a, provided off-center, in the circumferential wall of the catheter. The secondary lumens 1122a, 1124a are provided with a lock line or lock lines 1152 which couples with a fixation element locking mechanism 1150 on the fixation device 1144. The locking mechanism 1150 may be configured to lock the fixation elements 1145 in position when actuated by the lock line(s) 1152. A locking mechanism 1150, along with the associated lumens 1122, 1124 and lock line(s) 1152, is optional. The fixation elements 1145 may be held in a desired position in other ways, including by biasing the fixation elements to or towards a closed position, for example using a spring mechanism, or by forming the fixation elements from a shape-memory material, such as Nitinol.

The end member 1112 includes openings corresponding to the lumens of the delivery catheter 1110: a main lumen opening 1126, gripper line openings 1118b, 1120b, and lock line openings 1122b, 1124b. The end member 1112 is also coupled with a coupling member 1142 (shown schematically in FIG. 43) for releasably coupling the delivery catheter 1110 to a fixation device. The coupling member 1142 is disposed centrally on the end member 1112 and includes a central opening to allow passage of the actuatable member 1116 therethrough.

The end member 1112 may be provided as a press-fit assembly which may be mounted onto the distal end of the delivery catheter 1110. The end member 1112 may optionally be provided with a radiopaque ring, so that the delivery catheter 1110 may be tracked using known imaging techniques.

FIG. 42 illustrates a cross-sectional view of a delivery catheter 1130 which includes a positioning device. The cross section is taken adjacent the distal end of the catheter 1130. The delivery catheter 1130 is shown as including the features of delivery catheter 1110, but it will be appreciated that one or more or all of the optional features described herein may be omitted or substituted with alternatives.

The delivery catheter 1130 is shown with four positioning device lumens 1132a-d. A first pair of positioning device lumens 1132a-b is provided on either side of a first gripping line lumen 1118. A second pair of positioning device lumens 1132c-d is provided on either side of a second gripping line lumen 1120. In an alternative arrangement, the pairs of positioning device lumens may be provided on either side of a respective lock line lumen 1122, 1124. This arrangement is shown in FIG. 43. In still further alternatives, the delivery catheter 1130 may include one positioning device lumen, two positioning device lumens, three positioning device lumens, four positioning device lumens, or more than four positioning device lumens. The positioning device and associated positioning member(s) may be configured and operated in accordance with any of the arrangements described herein. An end member provided with such a delivery catheter may be correspondingly modified to include openings which correspond to the lumens provided in the delivery catheter, as will be appreciated.

As noted, FIG. 43 shows schematically a fixation device 1144 releasably coupled to a coupling member 1142 of a delivery catheter which is configured with a positioning device as described herein. The end member 1140 of the delivery catheter is also shown in a blown-up, cross-sectional view. The positioning device and associated positioning member(s) may be configured and operated in accordance with any of the arrangements described herein. Providing the pairs of positioning device lumens 1132a, b and 1132 c, d on either side of a respective lock line lumen 1122, 1124 offers an advantage that the positioning member(s) may be deployed perpendicular to the opening and closing lines of the fixation elements and/or the gripping elements. This may allow for a pacemaker lead to be engaged and repositioned without interfering with the actuation and/or deployment of the fixation elements and/or the gripping elements.

In each or any of the above examples, the first lumen of the respective catheter may be a main lumen of the catheter. The first lumen may be a central lumen, located substantially centrally in the catheter. The first lumen may have a larger diameter than other lumens in the catheter.

Any of the positioning devices described herein, including those with one positioning member and those with more than one positioning member, may be provided with an atrioventricular valve modification device delivery system, including any of the atrioventricular valve modification device delivery systems described herein. Examples below are described in relation to a fixation device delivery system, which may include any of the fixation device delivery systems described herein. It will be appreciated that the systems may alternatively be provided with another atrioventricular valve modification device, with appropriate modifications being made accordingly.

Any of the positioning devices described herein may be configured to be deployed from, or through, a delivery catheter of a delivery system. In particular, the positioning device may be provided through one or more than one lumen of the delivery catheter, for example with the or each positioning member provided through a respective lumen. One such lumen may be a central lumen of a delivery catheter. Alternatively, or additionally, one such lumen may be an off-center, or peripheral, lumen of a delivery catheter. In implementations in which more than one lumen is used, the lumens may include a central lumen and an off-center lumen, or multiple off-center lumens.

The or each off-center lumen may be dedicated for the positioning device. Alternatively, the positioning device may share one or more than one lumen with another component or other components. Such other component may optionally include a line for a proximal element or gripper, or a line for a locking mechanism, of a fixation device.

In implementations in which the central lumen of a delivery catheter is used, the positioning device may share the central lumen with another component or other components. For example, an actuation mechanism, such as an actuator rod, for actuating fixation elements of a fixation device, may be provided through the central lumen. The or each positioning member of a positioning device may also be provided through the central lumen.

As discussed herein, the delivery catheter may be releasably coupled with a fixation device by means of a coupling member, such as coupling member 806 or coupling member 1142 described herein. In some implementations, the positioning device may be provided through the coupling member. In this way, the positioning device may be deployed at a closer location to the fixation device than when deployed from a distal end of the delivery catheter.

As noted, the positioning device may be configured to deploy from a distal end of the delivery catheter and assume a deployed configuration to function as described herein. Alternatively, or additionally, the positioning device may be configured to deploy from a distal end of a coupling mechanism of the delivery catheter and then assume a deployed configuration to function as described herein. Optionally, in either configuration, the positioning device may additionally pass through a portion of the fixation device which is to be deployed. The positioning device may pass through and be deployed from the fixation device on one or both sides of the fixation device. When deploying from either side, or both sides, of the fixation device, the positioning device may be configured with one positioning member configured to be deployed from one of the fixation elements of the fixation device. Alternatively, when deploying from either side, or both sides, of the fixation device, the positioning device may be configured with first and second positioning members on the or each side, the first and second positioning members being configured to be deployed from a respective one of first and second fixation elements of the fixation device. In this way, the positioning device may include one positioning member, two positioning members, three positioning members, or four positioning members.

FIGS. 44-47 illustrate some different configurations of such delivery systems. The delivery system in these examples includes a fixation device 1160, releasably coupled with a delivery catheter 1162 by a coupling member 1164. The fixation device includes first and second fixation elements 1166, 1168. The fixation device may optionally include a covering. Additionally or alternatively, the fixation device may optionally include first and second gripper elements, which may be configured to cooperate with a respective one of the first and second fixation elements to engage and retain heart valve tissue therebetween. Additionally or alternatively still, the fixation device may optionally include a locking mechanism, which may be configured to hold or lock the fixation elements of the fixation device in one or more locked configurations. These and/or other components of the fixation device, the delivery catheter, and/or the coupling member may each be configured in any of the ways as described separately and/or in combination herein.

The delivery system is configured with a positioning device. In the delivery system illustrated in FIG. 44, a positioning device 1170 is configured to be deployed from the distal end of the delivery catheter 1162. The positioning device 1170 is configured to assume an operational or deployed configuration in a region between the distal end of the delivery catheter 1162 and the fixation device 1160. The positioning device 1170 is shown with first and second positioning members 1170a, b. Each positioning member 1170a, b may be provided within a respective, separate delivery catheter lumen. Alternatively, the positioning members 1170a, b may be provided within the same delivery catheter lumen.

In the delivery system illustrated in FIG. 45, a positioning device 1172 is provided from the distal end of the delivery catheter 1162 and configured to pass through a portion, or portions, of the fixation device 1160 for deployment from the fixation device portion(s). The positioning device 1172 is shown with first and second positioning members 1172a, b. Each positioning member 1172a, b may be provided within a respective, separate delivery catheter lumen. Alternatively, the positioning members 1172a, b may be provided within the same delivery catheter lumen. In the example shown in FIG. 45, a first positioning member 1172a is configured to pass through a first fixation element 1166 of the fixation device 1160. A second positioning member 1172b is configured to pass through a second fixation element 1168 of the fixation device 1160. The positioning members 1172a, b may be configured to be deployed from a respective outer end of the first and second fixation elements 1166, 1168. Alternatively, the positioning members 1172a, b may be configured to be deployed from a respective side portion of the first and second fixation elements 1166, 1168 (into or out of the page as viewed in FIG. 45). The positioning device 1172 is configured to assume an operational or deployed configuration in a region adjacent and/or to the side(s) of the fixation device 1160.

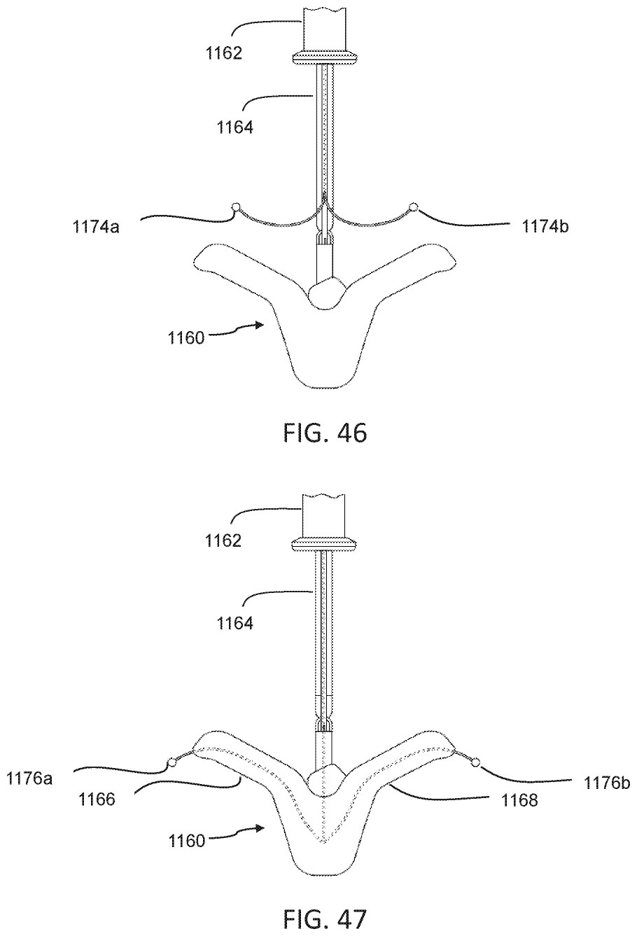

In the delivery system illustrated in FIG. 46, a positioning device 1174 is configured to be deployed from the distal end of the coupling member 1164. The positioning device is configured to assume an operational or deployed configuration in a region between the distal end of the coupling member 1164 and the fixation device 1160. The positioning device 1174 is shown with first and second positioning members 1174a, b. Each positioning member 1174a, b may be provided within a respective, separate delivery catheter lumen. Alternatively, the positioning members 1174a, b may be provided within the same delivery catheter lumen, for example a central lumen of the delivery catheter 1162.

In the delivery system illustrated in FIG. 47, a positioning device 1176 is provided from the distal end of the coupling member 1164 and configured to pass through a portion, or portions, of the fixation device 1160 for deployment from the fixation device portion(s). The positioning device 1176 is shown with first and second positioning members 1176a, b. Each positioning member 1176a, b may be provided within a respective, separate delivery catheter lumen. Alternatively, the positioning members 1176a, b may be provided within the same delivery catheter lumen, for example a central lumen of the delivery catheter 1162. In the example shown in FIG. 47, a first positioning member 1176a is configured to pass into the fixation device 1160 proximally, from the coupling member 1164. The first positioning member 1176a then passes through a first fixation element 1166 of the fixation device 1160. A second positioning member 1176b is configured to pass into the fixation device 1160 proximally, from the coupling member 1164. The second positioning member 1176b then passes through a second fixation element 1168 of the fixation device 1160. The positioning members 1176a, b may be configured to be deployed from a respective outer end of the first and second fixation elements 1166, 1168. Alternatively, the positioning members 1176a, b may be configured to be deployed from a respective side portion of the first and second fixation elements 1166, 1168 (into or out of the page as viewed in FIG. 47). Alternatively still, the positioning members 1176a, b may be configured to be deployed from a side of the central body region of the fixation device, or from a side portion of the inner ends of the first and second fixation elements 1166, 1168 (in each case, into or out of the page as viewed in FIG. 47). The positioning device 1176 is configured to assume an operational or deployed configuration in a region adjacent and/or to the side(s) of the fixation device 1160.

While the examples shown in FIGS. 44-47 illustrate positioning devices with first and second positioning members, each and any of the positioning devices may alternatively be provided with one positioning member, which may be configured in any of the described positions and configurations. In arrangements in which any of the described positioning devices include two positioning members, the positioning members may be provided on the same side of the delivery system or fixation device, or alternatively on opposite sides of the delivery system or fixation device. In still further arrangements, any of the positioning devices may include three positioning members, with two on one side and the third on the other side of the delivery system or fixation device. In further arrangements, any of the positioning devices may be provided with four positioning members, with two on each side of the delivery system or fixation device. Furthermore, a delivery system may optionally be configured with a first positioning device provided from the distal end of the delivery catheter and a second positioning device provided from the distal end of the coupling member. Such positioning devices may be configured in any of the ways described herein.

Any of the positioning devices 1170, 1172, 1174, 1176 and/or any of the individual or collective positioning members 1170a, b, 1172a, b, 1174a, b, 1176a, b may be configured structurally and/or functionally in any of the ways described herein. For example, any of the positioning devices 1170, 1172, 1174, 1176 and/or any of the individual or collective positioning members 1170a, b, 1172a, b, 1174a, b, 1176a, b may be configured with one or more of the materials, shapes, end structures, and/or functionality, including cooperation between multiple positioning members in arrangements provided with more than one positioning member, as described herein.

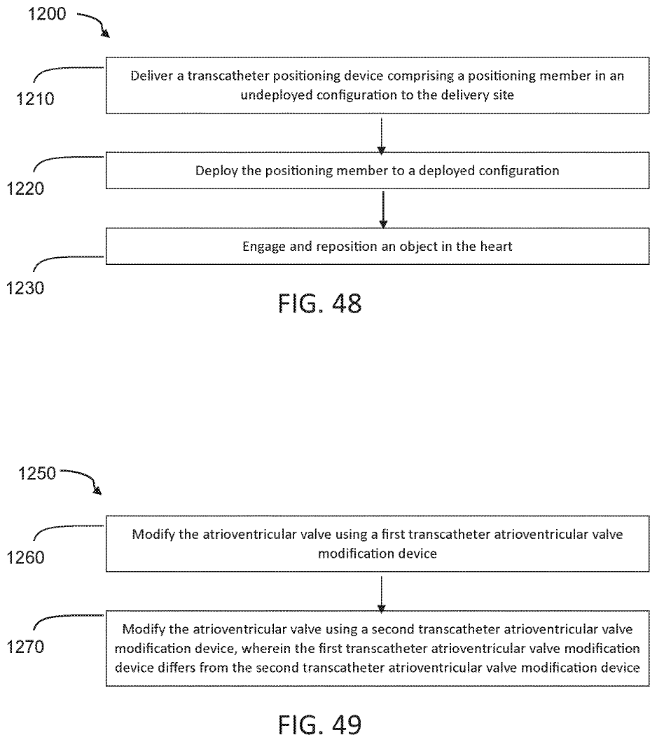

FIG. 48 illustrates a method 1200 for delivering a transcatheter atrioventricular valve modification device to an atrioventricular valve modification device delivery site in the heart of a patient. In step 1210, a transcatheter positioning device comprising a positioning member is delivered in an undeployed configuration to the delivery site. In step 1220, the positioning member is deployed to a deployed configuration. In step 1230, an object in the heart is engaged and repositioned.

The object may include a lead or leads of a pacemaker passing through the atrioventricular valve from the atrium to the ventricle associated with the atrioventricular valve. The object may further include one or more of a) one or more chordae tendineae, b) a papillary muscle, and/or c) a moderator band in the ventricle associated with the atrioventricular valve.

The method may further include the step of delivering an atrioventricular valve modification device to the delivery site while the positioning member is in the deployed configuration. The atrioventricular valve modification device may be a fixation device for engaging and approximating native leaflets of the atrioventricular valve. The atrioventricular valve modification device may alternatively be a prosthetic atrioventricular valve.

FIG. 49 illustrates a method 1250 for modifying a native atrioventricular valve. In step 1260, the atrioventricular valve is modified using a first transcatheter atrioventricular valve modification device. In step 1270, the atrioventricular valve is modified using a second transcatheter atrioventricular valve modification device, wherein the first transcatheter atrioventricular valve modification device differs from the second transcatheter atrioventricular valve modification device. The first atrioventricular valve modification device may correspond structurally to the second atrioventricular valve modification device, but the first atrioventricular valve modification device may be smaller than the second atrioventricular valve modification device. Alternatively, the first atrioventricular valve modification device may differ structurally from the second atrioventricular valve modification device. Optionally, the first atrioventricular valve modification device may differ structurally from and be smaller than the second atrioventricular valve modification device.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

The following Examples are illustrative of several embodiments of the present technology.

### EXAMPLES

Example 1. A method of implanting a prosthetic atrioventricular valve into a native atrioventricular valve of a patient, the method comprising:
delivering a fixation device to the native atrioventricular valve;
applying the fixation device to engage and approximate free edges of two native leaflets of the native atrioventricular valve;
after applying the fixation device, delivering the prosthetic atrioventricular valve to the native atrioventricular valve while the prosthetic atrioventricular valve is in a collapsed condition; and
deploying the prosthetic atrioventricular valve into the native atrioventricular valve so that the prosthetic atrioventricular valve expands into engagement with the native atrioventricular valve.

Example 2. The Example of Example 1, wherein prior to applying the fixation device, a native annulus of the native atrioventricular valve has an initial size, and after applying the fixation device, the native annulus of the native atrioventricular valve has a modified size, the modified size being smaller than the initial size.

Example 3. The method of Example 2, wherein the prosthetic atrioventricular valve is self-expandable and has an operational size in the absence of applied forced, the operational size of the prosthetic atrioventricular valve being too small to anchor to the native annulus of the atrioventricular valve when the native annulus has the initial size, but being sufficiently large to anchor to the native annulus of the atrioventricular valve when the native annulus has the modified size.

Example 4. The method of any of the preceding Examples, wherein the native atrioventricular valve is a native tricuspid valve, and applying the fixation device includes:
applying a first fixation device to engage and approximate free edges of a first native leaflet and a second native leaflet of the native tricuspid valve; and
applying a second fixation device to engage and approximate free edges of the first native leaflet and a third native leaflet of the native tricuspid valve.

Example 5. The method of any of the preceding Example, wherein the prosthetic atrioventricular valve includes a frame that has a waist portion, an atrial anchor flaring radially outward from the waist portion, and a ventricular anchor flaring radially outward from the waist portion, and wherein deploying the prosthetic atrioventricular valve into the native atrioventricular valve includes positioning the fixation device in a space between the atrial anchor and the ventricular anchor of the frame.

Example 6. The method of any of the preceding Examples, wherein the steps of applying the fixation device and deploying the prosthetic atrioventricular valve are performed as part of a single procedure performed in the same day.

Example 7. A method of implanting a prosthetic atrioventricular valve into a native atrioventricular valve of a patient, the method comprising:
delivering a first fixation device to the native atrioventricular valve;
applying the first fixation device to engage and approximate free edges of two native leaflets of the native atrioventricular valve;
delivering a second fixation device to the native atrioventricular valve;
applying the second fixation device to engage and approximate free edges of two native leaflets of the native atrioventricular valve;
delivering the prosthetic atrioventricular valve to the native atrioventricular valve while the prosthetic atrioventricular valve is in a collapsed condition; and
deploying the prosthetic atrioventricular valve into the native atrioventricular valve by inflating a balloon over which the native atrioventricular valve is crimped to expand the prosthetic atrioventricular valve against the first fixation device and against the second fixation device to anchor the prosthetic atrioventricular valve within the native atrioventricular valve.

Example 8. The method of Example 7, wherein the native atrioventricular valve is a native mitral valve, wherein applying the first fixation device includes engaging and approximating free edges of a native anterior leaflet and a native posterior leaflet of the native mitral valve at a first location, and applying the second fixation device includes engaging and approximating free edges of the native anterior leaflet and the native posterior leaflet of the native mitral valve at a second location, the first location being closer to an anterolateral commissure of the native mitral valve than is the second location, and the second location being closer to a posteromedial commissure of the native mitral valve than is the first location.

Example 9. The method of Example 7, wherein the native atrioventricular valve is a native tricuspid valve, wherein the two native leaflets engaged and approximated by the first fixation device is a first pair selected from (i) a first leaflet of the native tricuspid valve, (ii) a second leaflet of the native tricuspid valve, and (iii) a third leaflet of the native tricuspid valve, and;
the two native leaflets engaged and approximated by the second fixation device is a second pair selected from (i) the first leaflet of the native tricuspid valve, (ii) the second leaflet of the native tricuspid valve, and (iii) the third leaflet of the native tricuspid valve, the first pair being different than the second pair.

Example 10. The method of Example 9, further comprising:
delivering a third fixation device to the native tricuspid valve; and
applying the third fixation device to engage and approximate free edges of two native leaflets of the native tricuspid valve.

Example 11. The method of Example 10, wherein the two native leaflets engaged and approximated by the third fixation device is a third pair selected from (i) the first leaflet of the native tricuspid valve, (ii) the second leaflet of the native tricuspid valve, and (iii) the third leaflet of the native tricuspid valve, the third pair being different than both the first pair and the second pair.

Example 12. The method of any of Examples 7-11, wherein the steps of applying the first fixation device, applying the second fixation device, and deploying the prosthetic atrioventricular valve are performed as part of a single procedure performed in the same day.

Example 13. A method of implanting a prosthetic tricuspid valve into a native tricuspid valve of a patient, the method comprising:
delivering a fixation device to the native tricuspid valve;
applying the fixation device to engage and approximate free edges of (i) a septal leaflet of the native tricuspid valve and (ii) a second leaflet of the native tricuspid valve, the second leaflet being either (a) an anterior leaflet of the native tricuspid valve or (b) a posterior leaflet of the native tricuspid valve;
after applying the fixation device, delivering the prosthetic tricuspid valve to the native tricuspid valve while the prosthetic tricuspid valve is in a collapsed condition; and
deploying the prosthetic tricuspid valve into the native tricuspid valve by expanding the prosthetic tricuspid valve into engagement with the native tricuspid valve, such that no structure of the prosthetic tricuspid valve contacts an atrioventricular (“AV”) node of the patient.

Example 14. The method of Example 13, wherein applying the fixation device includes:
applying a first fixation device to engage and approximate free edges of (i) the septal leaflet of the native tricuspid valve and (ii) the anterior leaflet of the native tricuspid valve; and
applying a second fixation device to engage and approximate free edges of (i) the septal leaflet of the native tricuspid valve and (ii) the posterior leaflet of the native tricuspid valve.

Example 15. The method of Examples 13 or 14, wherein the steps of applying the fixation device and deploying the prosthetic tricuspid valve are performed as part of a single procedure performed in the same day.

Example 16. A method of implanting a prosthetic mitral valve into a native mitral valve of a patient, the method comprising:
delivering a fixation device to the native mitral valve;
applying the fixation device to engage and approximate free edges of (i) an anterior leaflet of the native mitral valve and (ii) a posterior leaflet of the native mitral valve;
after applying the fixation device, delivering the prosthetic mitral valve to the native mitral valve while the prosthetic mitral valve is in a collapsed condition; and
deploying the prosthetic mitral valve into the native mitral valve by expanding the prosthetic mitral valve into engagement with the native mitral valve, such that no structure of the prosthetic mitral valve obstructs blood flowing through a left ventricular outflow tract (“LVOT”) of the patient.

Example 17. The method of Example 16, wherein the steps of applying the fixation device and deploying the prosthetic mitral valve are performed as part of a single procedure performed in the same day.

Example 18. A method of implanting a prosthetic atrioventricular valve into a native atrioventricular valve of a heart of a patient, wherein the patient has a pacemaker lead extending from an atrium of the heart to a ventricle of the heart through the native atrioventricular valve, the method comprising:
delivering a fixation device to the native atrioventricular valve;
applying the fixation device to engage and approximate free edges of two native leaflets of the native atrioventricular valve so that a portion of the pacemaker lead becomes trapped between the fixation device and an adjacent commissure of the native atrioventricular valve;
while the pacemaker lead is trapped, delivering the prosthetic atrioventricular valve to the native atrioventricular valve while the prosthetic atrioventricular valve is in a collapsed condition; and
deploying the prosthetic atrioventricular valve into the native atrioventricular valve so that the prosthetic atrioventricular valve expands into engagement with the native atrioventricular valve.

Example 19. The method of Example 18, wherein the native atrioventricular valve is a native tricuspid valve, the two native leaflets engaged and approximated by the fixation device are a septal leaflet and a posterior leaflet of the native tricuspid valve, and the commissure is a commissure connecting the septal leaflet to the posterior leaflet.

Example 20. The method of Example 18 or 19, wherein the steps of applying the fixation device and deploying the prosthetic atrioventricular valve are performed as part of a single procedure performed in the same day.

Example 21. The method of any of Examples 18-20, wherein the method includes implanting a pacemaker into the patient so that the pacemaker lead extends from the atrium to the ventricle through the native atrioventricular valve, and the steps of (i) implanting the pacemaker, (ii) applying the fixation device, and (iii) deploying the prosthetic atrioventricular valve are performed as part of a single procedure performed in the same day.

Example 22. A method of implanting a prosthetic atrioventricular valve into a native atrioventricular valve of a heart of a patient, the method comprising:
delivering the prosthetic atrioventricular valve to the native atrioventricular valve while the prosthetic atrioventricular valve is in a collapsed condition; and
deploying the prosthetic atrioventricular valve into the native atrioventricular valve so that the prosthetic atrioventricular valve expands into engagement with the native atrioventricular valve,
wherein deploying the prosthetic atrioventricular valve includes (i) positioning a first anchor arm of the prosthetic atrioventricular valve in a first gap space between (a) a first fixation device that engages and approximates free edges of two native leaflets of the native atrioventricular valve and (b) a first native commissure adjacent to the fist fixation device, and (ii) positioning a second anchor arm of the prosthetic atrioventricular valve in a second gap space between (a) a second fixation device that engages and approximates free edges of two native leaflets of the native atrioventricular valve and (b) a second native commissure adjacent to the second fixation device.

Example 23. The method of Example 22, wherein after the prosthetic atrioventricular valve is deployed, the first anchor arm include a first base connected to a ventricular end portion of the prosthetic atrioventricular valve, a first terminal end, and a first middle portion between the first base and the first terminal end.

Example 24. The method of Example 23, wherein after the prosthetic atrioventricular valve is deployed, the first base of the first anchor arm is positioned on an outflow side of the native atrioventricular valve, the first terminal end of the first anchor arm is positioned on an inflow side of the native atrioventricular valve, and the first middle portion of the first anchor arm is positioned within the first gap space.

Example 25. The method of Example 24, wherein after the prosthetic atrioventricular valve is deployed, the first terminal end of the first anchor arm is in contact with the first fixation device.

Example 26. The method of any of Examples 22-25, further comprising:
prior to delivering the prosthetic atrioventricular valve to the native atrioventricular valve, delivering the first fixation device to the native atrioventricular valve and applying the first fixation device to engage and approximate free edges of two native leaflets of the native atrioventricular valve, and delivering the second fixation device to the native atrioventricular valve and applying the second fixation device to engage and approximate free edges of two native leaflets of the native atrioventricular valve,
wherein the steps of (i) applying the first fixation device, (ii) applying the second fixation device, and (iii) deploying the prosthetic atrioventricular valve are performed as part of a single procedure performed in the same day.

Example 27. The method of any of Examples 22-26, further comprising, after positioning the first anchor arm of the prosthetic atrioventricular valve in the first gap space, delivering an occluder to the native atrioventricular valve and deploying the occluder within the first gap space.

Example 28. A transcatheter positioning device for an atrioventricular valve modification device delivery system, the positioning device comprising a positioning member having an undeployed configuration for delivery of the positioning device to an atrioventricular valve modification device delivery site in the heart of a patient, and a deployed configuration for the positioning member to engage and reposition an object in the heart.

Example 29. The transcatheter positioning device of Example 28, wherein the object comprises a pacemaker lead or leads passing through the atrioventricular valve from the atrium associated with the atrioventricular valve to the ventricle associated with the atrioventricular valve.

Example 30. The transcatheter positioning device of Example 28 or 29, wherein the object comprises one or more of a) one or more chordae tendineae, b) a papillary muscle, and/or c) a moderator band in the ventricle associated with the atrioventricular valve.

Example 31. The transcatheter positioning device of any of Examples 28-30, wherein the positioning member comprises a shape-memory material configured to assume the deployed configuration when the positioning member is unconstrained.

Example 32. The transcatheter positioning device of any of Examples 28-31, wherein the delivery system further comprises a delivery catheter configured to pass through the patient's vasculature to the delivery site, the delivery catheter comprising:
a first lumen for delivery of the atrioventricular valve modification device to the delivery site; and
a second lumen for constraining the positioning member in the undeployed configuration and from which the positioning member is configured to be deployed to assume the deployed configuration.

Example 33. The transcatheter positioning device of any of Examples 28-32, wherein the delivery system further comprises a guide catheter configured to pass through the patient's vasculature to the delivery site, the guide catheter comprising:
a first lumen for passage therethrough of a delivery catheter for delivery of the atrioventricular valve modification device to the delivery site; and
a second lumen for constraining the positioning member in the undeployed configuration and from which the positioning member is configured to be deployed to assume the deployed configuration.

Example 34. The transcatheter positioning device of Example 32 or 33, wherein the first lumen is a central lumen and the second lumen is an off-center lumen.

Example 35. The transcatheter positioning device of any of Examples 32-34, wherein the positioning member is configured to be retracted back into the second lumen for withdrawal from the heart of the patient with the delivery system.

Example 36. The transcatheter positioning device of any of Examples 28-35, wherein the delivery system comprises a control interface at a proximal end, the control interface comprising a positioning device control mechanism for deploying the positioning member between the undeployed configuration and the deployed configuration.

Example 37. The transcatheter positioning device of Example 36, wherein the positioning device control mechanism comprises a rotational control mechanism for rotating the positioning member.

Example 38. The transcatheter positioning device of any of Examples 28-37, wherein in the deployed configuration the positioning member comprises a hook shaped portion.

Example 39. The transcatheter positioning device of any of Examples 28-38, wherein in the deployed configuration the positioning member comprises a loop shaped portion.

Example 40. The transcatheter positioning device of any of Examples 28-39, wherein in the deployed configuration the positioning member comprises a double loop shaped portion.

Example 41. The transcatheter positioning device of any of Examples 28-40, wherein in the deployed configuration the positioning member comprises a heart shaped portion.

Example 42. The transcatheter positioning device of any of Examples 28-41, wherein in the deployed configuration the positioning member comprises a wishbone shaped portion.

Example 43. The transcatheter positioning device of any of Examples 28-42, wherein in the deployed configuration the positioning member comprises an S shaped portion.

Example 44. The transcatheter positioning device of any of Examples 28-43, wherein in the deployed configuration the positioning member comprises first and second guide portions and an engagement portion therebetween.

Example 45. The transcatheter positioning device of any of Examples 28-44, wherein in the deployed configuration the positioning member comprises a FIG. 3 shaped portion.

Example 46. The transcatheter positioning device of any of Examples 28-45, wherein in the deployed configuration the positioning member comprises a FIG. 8 shaped portion.

Example 47. The transcatheter positioning device of any of Examples 28-46, wherein in the deployed configuration the positioning member comprises a coil or helix shaped portion.

Example 48. The transcatheter positioning device of any of Examples 28-47, wherein in the deployed configuration the positioning member comprises a spiral shaped portion.

Example 49. The transcatheter positioning device of Example 48, wherein a radius of curvature of the spiral shaped portion decreases as the positioning member is deployed.

Example 50. The transcatheter positioning device of any of Examples 28-49, wherein the positioning device comprises a first end and the positioning member is provided at the first end.

Example 51. The transcatheter positioning device of any of Examples 28-49, wherein the positioning device comprises first and second ends and the positioning member is provided partway between the first and second ends.

Example 52. The transcatheter positioning device of any of Examples 28-51, wherein the positioning device comprises two positioning members configured to cooperate with each other in the deployed configuration.

Example 53. The transcatheter positioning device of Example 52, wherein in the deployed configuration the two positioning members cooperate to form the shaped portion of any of Examples 38-49.

Example 54. The transcatheter positioning device of any of Examples 52-53, wherein in the deployed configuration the two positioning members cooperate to form one of a U shaped portion, a V shaped portion, an X shaped portion, or a Y shaped portion.

Example 55. The transcatheter positioning device of any of Examples 52-54, wherein each of the two positioning members comprises a respective magnet and in the deployed configuration the two positioning members are releasably coupled together by the magnets.

Example 56. The transcatheter positioning device of any of Examples 28-55, wherein the positioning member is a first positioning member and the positioning device comprises a second positioning member, the second positioning member being configured according to the positioning member of any of Examples 31-51.

Example 57. The transcatheter positioning device of any of Examples 52-56, wherein the second positioning member is deployable independently of the first positioning member.

Example 58. The transcatheter positioning device of any of Examples 28-57, wherein the atrioventricular valve modification device delivery system is configured to deliver an atrioventricular valve modification device to the delivery site while the positioning member is in the deployed configuration.

Example 59. The transcatheter positioning device of any of Examples 28-58, wherein the atrioventricular valve modification device comprises a first fixation device for engaging and approximating native leaflets of the atrioventricular valve.

Example 60. The transcatheter positioning device of Example 59, wherein in the deployed configuration the positioning member is configured to pass through one or more openings in the first fixation device.

Example 61. The transcatheter positioning device of Example 59 or 60, wherein the positioning member and the first fixation device are configured to cooperate during deployment of the first fixation device such that the positioning member engages and repositions the object in the heart as the first fixation device engages and approximates native leaflets of the atrioventricular valve.

Example 62. The transcatheter positioning device of any of Examples 59-61, wherein the positioning device comprises a first positioning member and a second positioning member and the first fixation device comprises a first fixation element and a second fixation element, wherein in the deployed configuration the first positioning member is configured to pass through one or more openings in the first fixation element and the second positioning member is configured to pass through one or more openings in the second fixation element.

Example 63. The transcatheter positioning device of Example 62, wherein the first and second positioning members are configured to be deployed on the same side of the first and second fixation elements and to cooperate during deployment of the first fixation device such that the first and second positioning members may engage and reposition the object in the heart as the first fixation device engages and approximates native leaflets of the atrioventricular valve.

Example 64. The transcatheter positioning device of Example 62 or 63, wherein the positioning device further comprises third and fourth positioning members, wherein in the deployed configuration the third positioning member is configured to pass through one or more openings in the first fixation element and the fourth positioning member is configured to pass through one or more openings in the second fixation element.

Example 65. The transcatheter positioning device of Example 64, wherein the third and fourth positioning members are configured to be deployed on the opposite side of the first and second fixation elements from the first and second positioning members and to cooperate during deployment of the first fixation device such that the third and fourth positioning members may engage and reposition the object in the heart as the first fixation device engages and approximates native leaflets of the atrioventricular valve.

Example 66. The transcatheter positioning device of any of Examples 28-65, wherein the atrioventricular valve modification device comprises a prosthetic atrioventricular valve.

Example 67. A method of delivering a transcatheter atrioventricular valve modification device to an atrioventricular valve modification device delivery site in the heart of a patient, the method comprising:
delivering a transcatheter positioning device comprising a positioning member in an undeployed configuration to the delivery site;
deploying the positioning member to a deployed configuration; and
engaging and repositioning an object in the heart.

Example 68. The method of Example 67, wherein the object comprises a pacemaker lead or leads passing through the atrioventricular valve from the atrium associated with the atrioventricular valve to the ventricle associated with the atrioventricular valve.

Example 69. The method of Example 67 or 68, wherein the object comprises one or more of a) one or more chordae tendineae, b) a papillary muscle, and/or c) a moderator band in the ventricle associated with the atrioventricular valve.

Example 70. The method of any of Examples 67-69, further comprising delivering an atrioventricular valve modification device to the delivery site while the positioning member is in the deployed configuration.

Example 71. The method of any of Examples 67-70, wherein the atrioventricular valve modification device comprises a fixation device for engaging and approximating native leaflets of the atrioventricular valve.

Example 72. The method of any of Examples 67-71, wherein the atrioventricular valve modification device comprises a prosthetic atrioventricular valve.

Example 73. The method of any of Examples 67-72, wherein the positioning device is configured according to any of Examples 28-66.

Example 74. An atrioventricular valve repair system comprising:
a first fixation device comprising first fixation elements configured to engage first heart tissue, the first heart tissue comprising one or more of a) first edge portions of two native leaflets of a native atrioventricular valve, the first edge portions being adjacent a first commissure defined between the two native leaflets, b) one or more chordae tendineae, c) a papillary muscle, and/or d) a moderator band;
a second fixation device comprising second fixation elements configured to engage second heart tissue, the second heart tissue comprising second edge portions of two native leaflets of the native atrioventricular valve, the second edge portions being located away from the first commissure; wherein the first fixation device is smaller than the second fixation device.

Example 75. The atrioventricular valve repair system of Example 74, wherein the first fixation elements have a first fixation element length and the second fixation elements have a second fixation element length, the first fixation element length being shorter than the second fixation element length.

Example 76. The atrioventricular valve repair system of Example 75, wherein the first fixation element length is 0.05 inches (1.3 mm) shorter than the second fixation element length, optionally wherein the first fixation element length is 0.265 inches (6.73 mm) and the second fixation element length is 0.315 inches (8.00 mm).

Example 77. The atrioventricular valve repair system of any of Examples 74-76, wherein the first fixation device further comprises first gripper elements for cooperating with a respective one of the first fixation elements to grip tissue therebetween, the first gripper elements having a first gripper element length, and the second fixation device further comprises second gripper elements for cooperating with a respective one of the second fixation elements to grip tissue therebetween, the second gripper elements having a second gripper element length, the first gripper element length being shorter than the second gripper element length.

Example 78. The atrioventricular valve repair system of Example 77, wherein the first gripper element length is 0.05 inches (1.3 mm) shorter than the second gripper element length, optionally wherein the first gripper element length is 0.256 inches (6.50 mm) and the second gripper element length is 0.306 inches (7.77 mm).

Example 79. The atrioventricular valve repair system of any of Examples 74-78, wherein the first gripper elements comprise a respective first plurality of frictional elements and the second gripper elements comprise a respective second plurality of frictional elements, the first plurality of frictional elements being lower than the second plurality of frictional elements.

Example 80. The atrioventricular valve repair system of Example 79, wherein the first plurality of frictional elements comprises one or two frictional element less than the second plurality of frictional elements, optionally wherein on one or both sides of the gripper elements the first plurality of frictional elements comprises three frictional elements and the second plurality of frictional elements comprises four frictional elements.

Example 81. The atrioventricular valve repair system of any of Examples 74-80, wherein the first gripper elements comprise a respective first plurality of frictional elements and the second gripper elements comprise a respective second plurality of frictional elements, the first plurality of frictional elements being spaced more closely together than the second plurality of frictional elements.

Example 82. The atrioventricular valve repair system of any of Examples 74-81, wherein the system further comprises:
a first delivery shaft for delivering the first fixation device, the first delivery shaft comprising a first coupling member at a distal end of the first delivery shaft for releasably coupling to the first fixation device, the first coupling member having a first coupling member length, and
a second delivery shaft for delivering the second fixation device, the second delivery shaft comprising a second coupling member at a distal end of the second delivery shaft for releasably coupling to the second fixation device, the second coupling member having a second coupling member length,
wherein the first coupling member length is shorter than the second coupling member length.

Example 83. The atrioventricular valve repair system of Example 82, wherein the first coupling member length is 0.05 inches (1.3 mm) shorter than the second coupling member length, optionally wherein the first coupling member length is 0.356 inches (9.04 mm) and the second coupling member length is 0.406 inches (10.3 mm).

Example 84. The atrioventricular valve repair system of any of Examples 74-83, wherein the first fixation device is configured to engage the first edge portions adjacent the first commissure to provide a gap between the first commissure and the first fixation device, the gap being configured to accommodate and secure a pacemaker lead or leads passing through the atrioventricular valve.

Example 85. The atrioventricular valve repair system of any of Examples 74-84, wherein the first fixation elements are configured to engage a pacemaker lead or leads passing through the atrioventricular valve, along with the one or more of b) the one or more chordae tendineae, c) the papillary muscle, and/or d) the moderator band, to secure the lead or leads of the pacemaker thereto.

Example 86. The atrioventricular valve repair system of any of Examples 74-85, wherein the second fixation device is configured to engage the second edge portions to approximate the native leaflets to reduce regurgitation through the atrioventricular valve.

Example 87. A method of modifying a native atrioventricular valve of a patient, the method comprising:
delivering a first fixation device to the native atrioventricular valve, the first fixation device comprising first fixation elements;
applying the first fixation elements to engage first edge portions of two native leaflets of the native atrioventricular valve, the first edge portions being adjacent a first commissure defined between the two native leaflets;
delivering a second fixation device to the native atrioventricular valve, the second fixation device comprising second fixation elements;
applying the second fixation elements to engage second edge portions of two native leaflets of the native atrioventricular valve, the second edge portions being located away from the first commissure.

Example 88. The method of Example 87, wherein the step of applying the first fixation elements comprises positioning the first fixation device to provide a gap between the first commissure and the first fixation device, the gap accommodating and/or securing a pacemaker lead or leads passing through the atrioventricular valve.

Example 89. The method of Example 87 or 88, further comprising positioning the lead or leads in the gap before applying the first fixation elements to engage the first edge portions.

Example 90. The method of any of Examples 88-89, further comprising positioning the lead or leads in the gap using a positioning member.

Example 91. The method of any of Examples 87-90, wherein the first fixation device is smaller than the second fixation device.

Example 92. The method of any of Examples 87-91, wherein the first fixation device is configured according to any of Examples 74-86.

Example 93. The method of any of Examples 87-92, wherein the second fixation device is configured according to any of Examples 74-86.

Example 94. The method of any of Examples 87-93, wherein the positioning member is configured according to any of Examples 28-66.

Example 95. A method of modifying a native atrioventricular valve of a patient, the native atrioventricular valve having a pacemaker lead or leads passing therethrough from the atrium associated with the atrioventricular valve to the ventricle associated with the atrioventricular valve, the method comprising:
delivering a first fixation device to the ventricle, the first fixation device comprising first fixation elements; and
applying the first fixation elements to engage the lead or leads of the pacemaker along with one or more of a) one or more chordae tendineae, b) a papillary muscle, and/or c) a moderator band in the ventricle, to secure the lead or leads of the pacemaker thereto.

Example 96. The method of Example 95, wherein the step of applying the first fixation elements comprises positioning the lead or leads at or adjacent a commissure defined between two native leaflets of the native atrioventricular valve.

Example 97. The method of Example 95 or 96, further comprising positioning the lead or leads using a positioning member.

Example 98. The method of any of Examples 95-97, further comprising delivering a second fixation device to the native atrioventricular valve, the second fixation device comprising second fixation elements; and
applying the second fixation elements to engage and approximate second edge portions of two native leaflets of the native atrioventricular valve.

Example 99. The method of any of Examples 95-98, further comprising delivering a prosthetic atrioventricular valve to the native atrioventricular valve while the prosthetic atrioventricular valve is in a collapsed condition; and
deploying the prosthetic atrioventricular valve into the native atrioventricular valve so that the prosthetic atrioventricular valve expands into engagement with the native atrioventricular valve.

Example 100. The method of Example 98, wherein the first fixation device is smaller than the second fixation device.

Example 101. The method of any of Examples 95-100, wherein the first fixation device is configured according to any of Examples 74-86.

Example 102. The method of any of Examples 98-101, wherein the second fixation device is configured according to any of Examples 74-86.

Example 103. The method of any of Examples 95-102, wherein the positioning member is configured according to any of Examples 28-66.

Example 104. A delivery system for delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the delivery system comprising:
a guide catheter configured to pass through the patient's vasculature towards the delivery site, the guide catheter comprising:
a first lumen for passage therethrough of a delivery catheter for delivery of an atrioventricular valve modification device to the delivery site;
a first steerable portion configured to deflect the guide catheter in the atrium from a first orientation not parallel to the inflow axis to a second orientation substantially parallel to the inflow axis for the guide catheter to pass through the atrioventricular valve from the atrium to the ventricle; and
a second steerable portion configured to deflect the guide catheter in the ventricle from the second orientation to a third orientation not parallel to the inflow axis.

Example 105. The delivery system of Example 104, wherein the second steerable portion is configured to deflect from a straight configuration to a bent configuration comprising a 90 ° bend.

Example 106. The delivery system of Example 104 or 105, wherein the second steerable portion is distal to the first steerable portion.

Example 107. The delivery system of any of Examples 104-106, wherein the third orientation is substantially perpendicular to the inflow axis.

Example 108. The delivery system of any of Examples 104-107, wherein the first orientation is substantially perpendicular to the inflow axis.

Example 109. The delivery system of any of Examples 104-108, wherein the second steerable portion comprises a radius of curvature of 0.5 cm to 1 cm.

Example 110. The delivery system of any of Examples 104-109, wherein the guide catheter comprises a distal end and the second steerable portion is 1 cm to 2 cm from the distal end.

Example 111. The delivery system of any of Examples 104-110, wherein the first steerable portion comprises a first steering mechanism configured to deflect the first steerable portion from the first orientation to the second orientation.

Example 112. The delivery system of Example 111, wherein the guide catheter comprises a first steering control interface at a proximal end of the guide catheter, the first steering control interface being configured to control the first steering mechanism.

Example 113. The delivery system of any of Examples 104-112, wherein the second steerable portion comprises a second steering mechanism configured to deflect the second steerable portion from the second orientation to the third orientation.

Example 114. The delivery system of Example 113, wherein the guide catheter comprises a second steering control interface at a proximal end of the guide catheter, the second steering control interface being configured to control the second steering mechanism.

Example 115. The delivery system of any of Examples 104-114, the delivery system further comprising:
a first delivery catheter configured for passage through the first lumen, the first delivery catheter being releasably coupled to a first fixation device comprising first fixation elements configured to engage first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

Example 116. The delivery system of any of Examples 104-115, wherein the first fixation elements are configured to engage a pacemaker lead or leads passing through the atrioventricular valve, along with the one or more of b) the one or more chordae tendineae, c) the papillary muscle, and/or d) the moderator band, to secure the lead or leads of the pacemaker thereto.

Example 117. The delivery system of any of Examples 104-116, the delivery system further comprising:
a second delivery catheter configured for passage through the first lumen, the second delivery catheter being releasably coupled to a second fixation device comprising second fixation elements configured to engage second heart tissue, the second heart tissue comprising second edge portions of two native leaflets of the native atrioventricular valve, the second edge portions being located away from the first commissure.

Example 118. The delivery system of Example 117, wherein the first delivery catheter is configured to extend further at the delivery site than the second delivery catheter.

Example 119. The delivery system of Example 117 or 118, wherein the first delivery catheter is configured to extend 2.5 cm to 6 cm further at the delivery site than the second delivery catheter.

Example 120. The delivery system of any of Examples 104-119, wherein the first fixation device is smaller than the second fixation device.

Example 121. The delivery system of any of Examples 104-120, wherein the first fixation device is configured according to any of Examples 74-86.

Example 122. The delivery system of any of Examples 104-121, wherein the second fixation device is configured according to any of Examples 74-86.

Example 123. The delivery system of any of Examples 104-122, the delivery system further comprising: a second delivery catheter configured for passage through the first lumen, the second delivery catheter being releasably coupled to a prosthetic atrioventricular valve.

Example 124. The delivery system of any of Examples 104-123, wherein the guide catheter and/or the first delivery catheter and/or the second delivery catheter comprises a respective positioning device.

Example 125. The delivery system of Example 124, wherein the or each positioning device is configured according to any of Examples 28-66.

Example 126. A method of delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the method comprising:
delivering a guide catheter through the patient's vasculature to the patient's heart;
deflecting a first steerable portion of the guide catheter in the atrium from a first orientation not parallel to the inflow axis to a second orientation substantially parallel to the inflow axis;
delivering the guide catheter through the atrioventricular valve from the atrium to the ventricle; and
deflecting a second steerable portion of the guide catheter in the ventricle from the second orientation to a third orientation not parallel to the inflow axis.

Example 127. The method of Example 126, wherein deflecting the second steerable portion comprises deflecting the second steerable portion from a straight configuration to a bent configuration comprising a 90 ° bend.

Example 128. The method of Example 126 or 127, wherein the third orientation is substantially perpendicular to the inflow axis.

Example 129. The method of any of Examples 126-128, wherein the first orientation is substantially perpendicular to the inflow axis.

Example 130. The method of any of Examples 126-129, wherein deflecting the first steerable portion comprises controlling a first steering mechanism using a first steering control interface at a proximal end of the guide catheter.

Example 131. The method of any of Examples 126-130, wherein deflecting the second steerable portion comprises controlling a second steering mechanism using a second steering control interface at a proximal end of the guide catheter.

Example 132. The method of any of Examples 126-131, wherein the guide catheter comprises a first lumen for passage therethrough of a delivery catheter for delivery of an atrioventricular valve modification device to the delivery site, the method further comprising delivering the first delivery catheter through the first lumen.

Example 133. The method of any of Examples 126-132, wherein the guide catheter comprises a first lumen for passage therethrough of a delivery catheter for delivery of an atrioventricular valve modification device to the delivery site, the method further comprising delivering the second delivery catheter through the first lumen.

Example 134. The method of Example 133, wherein the first delivery catheter is delivered through the first lumen, then withdrawn from the first lumen, and the second delivery catheter is then delivered through the first lumen.

Example 135. The method of Example 134, wherein, after withdrawal of the first delivery catheter, the second steerable portion of the guide catheter is deflected in the ventricle back to the second orientation before delivering the second delivery catheter through the first lumen.

Example 136. A delivery system for delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the delivery system comprising:
a first delivery catheter configured to pass through the patient's vasculature to the delivery site, the first delivery catheter comprising a first steerable delivery catheter portion configured to deflect the first delivery catheter in the ventricle from a first orientation substantially parallel to the inflow axis to a second orientation not parallel to the inflow axis for the first delivery catheter to be directed towards first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

Example 137. The delivery system of Example 136, the delivery system further comprising a guide catheter configured to pass through the patient's vasculature towards the delivery site, the guide catheter comprising:
a first lumen for passage therethrough of the first delivery catheter; and
a first steerable guide catheter portion configured to deflect the guide catheter in the atrium from a third orientation not parallel to the inflow axis to a fourth orientation substantially parallel to the inflow axis for the guide catheter to deliver the first delivery catheter to the atrium in the first orientation.

Example 138. The delivery system of Example 136 or 137, wherein the first steerable delivery catheter portion is configured to deflect from a straight configuration to a bent configuration comprising a 90 ° bend.

Example 139. The delivery system of any of Examples 136-138, wherein the second orientation is substantially perpendicular to the inflow axis.

Example 140. The delivery system of any of Examples 136-139, wherein the third orientation is substantially perpendicular to the inflow axis.

Example 141. The delivery system of any of Examples 136-140, wherein the first steerable delivery catheter portion comprises a radius of curvature of 0.5 cm to 1 cm.

Example 142. The delivery system of any of Examples 136-141, wherein the first delivery catheter comprises a distal end and the first steerable delivery catheter portion is 1 cm to 3 cm from the distal end.

Example 143. The delivery system of any of Examples 136-142, wherein the first steerable delivery catheter portion comprises a first delivery catheter steering mechanism configured to deflect the first steerable delivery catheter portion from the first orientation to the second orientation.

Example 144. The delivery system of Example 143, wherein the first delivery catheter comprises a first steering control interface at a proximal end of the first delivery catheter, the first steering control interface being configured to control the first delivery catheter steering mechanism.

Example 145. The delivery system of any of Examples 137-144, wherein the first steerable guide catheter portion comprises a first guide catheter steering mechanism configured to deflect the first steerable guide catheter portion from the third orientation to the fourth orientation.

Example 146. The delivery system of Example 145, wherein the guide catheter comprises a second steering control interface at a proximal end of the guide catheter, the second steering control interface being configured to control the first guide catheter steering mechanism.

Example 147. The delivery system of any of Examples 136-146, wherein the first delivery catheter is releasably coupled to a first fixation device comprising first fixation elements configured to engage the first heart tissue.

Example 148. The delivery system of Example 147, wherein the first fixation elements are configured to engage a pacemaker lead or leads passing through the atrioventricular valve, along with the one or more of b) the one or more chordae tendineae, c) the papillary muscle, and/or d) the moderator band, to secure the lead or leads of the pacemaker thereto.

Example 149. The delivery system of any of Examples 136-148, the delivery system further comprising:
a second delivery catheter configured to pass through the patient's vasculature to the delivery site, the second delivery catheter being releasably coupled to a second fixation device comprising second fixation elements configured to engage second heart tissue, the second heart tissue comprising second edge portions of two native leaflets of the native atrioventricular valve, the second edge portions being located away from the first commissure.

Example 150. The delivery system of Example 149, wherein the first delivery catheter is configured to extend further at the delivery site than the second delivery catheter.

Example 151. The delivery system of Example 149 or 150, wherein the first delivery catheter is configured to extend 2.5 cm to 6 cm further at the delivery site than the second delivery catheter.

Example 152. The delivery system of any of Examples 149-151, wherein the first fixation device is smaller than the second fixation device.

Example 153. The delivery system of any of Examples 147-152, wherein the first fixation device is configured according to any of Examples 74-86.

Example 154. The delivery system of any of Examples 149-153, wherein the second fixation device is configured according to any of Examples 74-86.

Example 155. The delivery system of any of Examples 136-154, the delivery system further comprising:
a second delivery catheter configured to pass through the patient's vasculature to the delivery site, the second delivery catheter being releasably coupled to a prosthetic atrioventricular valve.

Example 156. The delivery system of any of Examples 136-155, wherein the first delivery catheter and/or the guide catheter and/or the second delivery catheter comprises a respective positioning device.

Example 157. The delivery system of Example 156, wherein the or each positioning device is configured according to any of Examples 28-66.

Example 158. A method of delivering a transcatheter atrioventricular valve modification device to a delivery site within the heart of a patient, the heart comprising a native atrioventricular valve defining an inflow axis from the atrium associated with the atrioventricular valve to the ventricle associated with the atrioventricular valve, the method comprising:
delivering a first delivery catheter through the atrioventricular valve from the atrium to the ventricle in a first orientation substantially parallel to the inflow axis; and
deflecting a first steerable delivery catheter portion of the first delivery catheter in the ventricle from the first orientation to a second orientation not parallel to the inflow axis for the first delivery catheter to be directed towards first heart tissue, the first heart tissue comprising one or more of a) first edge portions of two native leaflets of the atrioventricular valve, the first edge portions being adjacent a first commissure defined between the two native leaflets, b) one or more chordae tendineae, c) a papillary muscle, and/or d) a moderator band.

Example 159. The method of Example 158, further comprising the steps of:
delivering a guide catheter through the patient's vasculature to the atrium;
deflecting the guide catheter in the atrium from a third orientation not parallel to the inflow axis to a fourth orientation substantially parallel to the inflow axis for the guide catheter to deliver the first delivery catheter to the atrium in the first orientation;
wherein the guide catheter comprises a first lumen for passage therethrough of the first delivery catheter, and the step of delivering the first delivery catheter comprises delivering the first delivery catheter through the first lumen into the atrium in the first orientation.

Example 160. The method of Example 158 or 159, wherein deflecting the first steerable delivery catheter portion comprises deflecting the first steerable delivery catheter portion from a straight configuration to a bent configuration comprising a 90 ° bend.

Example 161. The method of any of Examples 158-160, wherein the second orientation is substantially perpendicular to the inflow axis.

Example 162. The method of any of Examples 158-161, wherein the third orientation is substantially perpendicular to the inflow axis.

Example 163. The method of any of Examples 158-162, wherein deflecting the first steerable delivery catheter portion comprises controlling a first delivery catheter steering mechanism using a first steering control interface at a proximal end of the delivery catheter.

Example 164. The method of any of Examples 159-163, wherein deflecting the guide catheter comprises controlling a first guide catheter steering mechanism using a second steering control interface at a proximal end of the guide catheter.

Example 165. The method of any of Examples 158-164, wherein the first delivery catheter is releasably coupled to a first fixation device comprising first fixation elements, the method further comprising engaging the first fixation elements with the first heart tissue.

Example 166. The method of any of Examples 158-165, further comprising trapping a pacemaker lead or leads passing through the atrioventricular valve between the first commissure and the first fixation device.

Example 167. The method of Example 165 or 166, further comprising engaging the first fixation elements with a pacemaker lead or leads passing through the atrioventricular valve along with the one or more of b) the one or more chordae tendineae, c) the papillary muscle, and/or d) the moderator band, to secure the lead or leads of the pacemaker thereto.

Example 168. The method of any of Examples 158-167, further comprising delivering a second delivery catheter through the atrioventricular valve from the atrium to the ventricle in the first orientation;
wherein the second delivery catheter is releasably coupled to a second fixation device comprising second fixation elements, the method further comprising engaging the second fixation elements with second heart tissue, the second heart tissue comprising second edge portions of two native leaflets of the native atrioventricular valve, the second edge portions being located away from the first commissure.

Example 169. The method of Example 168, wherein the first fixation device is smaller than the second fixation device.

Example 170. The method of any of Examples 165-169, wherein the first fixation device is configured according to any of Examples 74-86.

Example 171. The method of any of Examples 168-170, wherein the second fixation device is configured according to any of Examples 74-86

Example 172. The method of any of Examples 158-171, further comprising delivering a second delivery catheter to the atrium in the first orientation;
wherein the second delivery catheter is releasably coupled to a prosthetic atrioventricular valve, the method further comprising deploying the prosthetic atrioventricular valve into the native atrioventricular valve so that the prosthetic atrioventricular valve expands into engagement with the native atrioventricular valve.

Example 173. The method of any of Examples 158-172, wherein the first delivery catheter is configured according to any of Examples 136-157.

Example 174. The method of any of Examples 158-173, wherein the second delivery catheter is configured according to any of Examples 149-157.

Example 175. The method of any of Examples 158-174, further comprising positioning the lead or leads using a positioning device.

Example 176. The method of Example 175, wherein the positioning device is configured according to any of Examples 28-66.

Example 177. A method of modifying a native atrioventricular valve, the method comprising:
modifying the atrioventricular valve using a first transcatheter atrioventricular valve modification device;
modifying the atrioventricular valve using a second transcatheter atrioventricular valve modification device;
wherein the first transcatheter atrioventricular valve modification device differs from the second transcatheter atrioventricular valve modification device.

Example 178. The method of Example 177, wherein the first atrioventricular valve modification device corresponds structurally to the second atrioventricular valve modification device, wherein the first atrioventricular valve modification device is smaller than the second atrioventricular valve modification device.

Example 179. The method of Example 177 or 178, wherein the first atrioventricular valve modification device differs structurally from the second atrioventricular valve modification device.

Example 180. The method of any of Examples 177-179, further comprising any of the method steps in accordance with any of Examples 67-73, 87-103, 126-135, and 158-176.

1. A method of implanting a prosthetic atrioventricular valve into a native atrioventricular valve of a heart of a patient, wherein the patient has a pacemaker lead extending from an atrium of the heart to a ventricle of the heart through the native atrioventricular valve, the method comprising:
delivering a fixation device to the native atrioventricular valve;
applying the fixation device to engage and approximate free edges of two native leaflets of the native atrioventricular valve so that a portion of the pacemaker lead becomes trapped between the fixation device and an adjacent commissure of the native atrioventricular valve;
while the pacemaker lead is trapped, delivering the prosthetic atrioventricular valve to the native atrioventricular valve while the prosthetic atrioventricular valve is in a collapsed condition; and
deploying the prosthetic atrioventricular valve into the native atrioventricular valve so that the prosthetic atrioventricular valve expands into engagement with the native atrioventricular valve.

2. The method of claim 1, wherein the native atrioventricular valve is a native tricuspid valve, the two native leaflets engaged and approximated by the fixation device are a septal leaflet and a posterior leaflet of the native tricuspid valve, and the commissure is a commissure connecting the septal leaflet to the posterior leaflet.

3. The method of claim 1, wherein the steps of applying the fixation device and deploying the prosthetic atrioventricular valve are performed as part of a single procedure performed in the same day.

4. The method of claim 1, wherein the method includes implanting a pacemaker into the patient so that the pacemaker lead extends from the atrium to the ventricle through the native atrioventricular valve, and the steps of (i) implanting the pacemaker, (ii) applying the fixation device, and (iii) deploying the prosthetic atrioventricular valve are performed as part of a single procedure performed in the same day.

5. A method of modifying a native atrioventricular valve within a heart of a patient, the native atrioventricular valve having a pacemaker lead or leads passing therethrough from the atrium associated with the atrioventricular valve to the ventricle associated with the atrioventricular valve, the method comprising:
delivering a first fixation device to the ventricle, the first fixation device comprising first fixation elements; and
applying the first fixation elements to engage the lead or leads of the pacemaker along with one or more of a) one or more chordae tendineae, b) a papillary muscle, and/or c) a moderator band in the ventricle, to secure the lead or leads of the pacemaker thereto.

6. The method of claim 5, wherein the step of applying the first fixation elements comprises positioning the lead or leads at or adjacent a commissure defined between two native leaflets of the native atrioventricular valve.

7. The method of claim 5, further comprising delivering a second fixation device to the native atrioventricular valve, the second fixation device comprising second fixation elements; and
applying the second fixation elements to engage and approximate second edge portions of two native leaflets of the native atrioventricular valve.

8. The method of claim 7, wherein the first fixation device is smaller than the second fixation device.

9. The method of claim 7, wherein the first fixation elements have a first fixation element length and the second fixation elements have a second fixation element length, the first fixation element length being shorter than the second fixation element length.

10. The method of claim 9, wherein the first fixation element length is 0.05 inches (1.3 mm) shorter than the second fixation element length.

11. The method of claim 9, wherein the first fixation element length is 0.265 inches (6.73 mm) and the second fixation element length is 0.315 inches (8.00 mm).

12. The method of claim 9, wherein the first fixation device further comprises first gripper elements for cooperating with a respective one of the first fixation elements to grip tissue therebetween, the first gripper elements having a first gripper element length, and the second fixation device further comprises second gripper elements for cooperating with a respective one of the second fixation elements to grip tissue therebetween, the second gripper elements having a second gripper element length, the first gripper element length being shorter than the second gripper element length.

13. The method of claim 12, wherein the first gripper element length is 0.05 inches (1.3 mm) shorter than the second gripper element length.

14. The method of claim 12, wherein the first gripper element length is 0.256 inches (6.50 mm) and the second gripper element length is 0.306 inches (7.77 mm).

15. The method of claim 5, further comprising positioning the lead or leads using a positioning member.

16. The method of claim 15, wherein the positioning member has an undeployed configuration for delivery, and a deployed configuration for the positioning member to engage and reposition the lead or leads in the heart.

17. The method of claim 16, wherein the positioning member comprises a shape-memory material configured to assume the deployed configuration when the positioning member is unconstrained.

18. The method of claim 17, wherein in the deployed configuration the positioning member comprises a hook shaped portion, a loop shaped portion, or a double loop shaped portion.

19. The method of claim 17, wherein in the deployed configuration the positioning member comprises a heart shaped portion, a wishbone shaped portion, or an S shaped portion.

20. A method of modifying a native atrioventricular valve, the method comprising:
modifying the atrioventricular valve using a first transcatheter atrioventricular valve modification device;
modifying the atrioventricular valve using a second transcatheter atrioventricular valve modification device;
wherein the first transcatheter atrioventricular valve modification device differs from the second transcatheter atrioventricular valve modification device.
